SEQUENCE LISTING (126 sequences)

SEQ ID NO: 1 - DNA, 2099 bp, Artificial Sequence
  Left TALE 1755
atggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtcgaacattggagggaaacaagcattggagactgtccaacggctccttcccgtgttgtgtcaagcccacggtttgacgcctgcacaagtggtcgccatcgcctccaatattggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaagcaacatcggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaagcaacatcggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcatcccacgacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgacacccgaacaggtggtcgccattgctaataataacggaggacggccagcctt

SEQ ID NO: 2 - protein, 699 aa, Artificial Sequence
  Left TALE 1755
MVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASHDGGKQALETVQRLLPVLCQDHGLTPEQVVAIANNNGGRPA

SEQ ID NO: 3 - DNA, 2099 bp, Artificial Sequence
  Right TALE 1756
atggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctccaatattggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgccaacaacaacggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaagcaatgggggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgccaacaacaacggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcatcccacgacggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgacacccgaacaggtggtcgccattgctaataataacggaggacggccagcctt

SEQ ID NO: 4 - protein, 699 aa, Artificial Sequence
  Right TALE 1756
MVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIASHDGGKQALETVQRLLPVLCQDHGLTPEQVVAIANNNGGRPA

SEQ ID NO: 5 - DNA, 18 bp, Artificial Sequence
  Left Target
tggaagactgagtgcccg

SEQ ID NO: 6 - DNA, 18 bp, Artificial Sequence
  Right Target
tggcaggcggtgggcgcg

SEQ ID NO: 7 - DNA, 4420 bp, Artificial Sequence
  Donor molecule
ccgccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcatggcccaacttgtttattgcagcttataatggttacaaataaagcaatagcatcacaaatttcacaaataaagcatttttttcactgcattctagttgtggtttgtccaaactcatcaatgtatcttatcatgtctggatctcctgcagataacttcgtatagcatacattatacgaagttatattaagggttccggatctcgaccagcttctgatggaattagaacttggcaaaacaatactgagaatgaagtgtatgtggaacagaggctgctgatctcgttcttcaggctatgaaactgacacatttggaaaccacagtacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcatcccggggatctgatatcatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtaagcttcagctgctcgagatctagatggatgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccacccttcccagctgctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaatttcgaggtcgagatcctaagcttggctggacgtaaactcctcttcagacctaataacttcgtatagcatacattatacgaagttatgtcgacgcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctg

SEQ ID NO: 8 - DNA, 2242 bp, Artificial Sequence
  Left homology
ccgccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacgg

SEQ ID NO: 9 - DNA, 202 bp, Artificial Sequence
  Right homology
cgcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctg

SEQ ID NO: 10 - DNA, 18 bp, Artificial Sequence
  Left target
tttcggccaggctgaggc

SEQ ID NO: 11 - DNA, 18 bp, Artificial Sequence
  Right target
ttcccaggcctgcagttt

SEQ ID NO: 12 - DNA, 18 bp, Artificial Sequence
  Left target
tccgagcgtgggtctccg

SEQ ID NO: 13 - DNA, 18 bp, Artificial Sequence
  Right target
tagggggcgggcccggat

SEQ ID NO: 14 - DNA, 17 bp, Artificial Sequence
  Left target
tccagggcctggacagg

SEQ ID NO: 15 - DNA, 16 bp, Artificial Sequence
  Right target
tcggggtcctcctgtc

SEQ ID NO: 16 - DNA, 17 bp, Artificial Sequence
  Left target
tggtgctgcctgtccaa

SEQ ID NO: 17 - DNA, 17 bp, Artificial Sequence
  Right target
tggagccgcctcagccg

SEQ ID NO: 18 - DNA, 18 bp, Artificial Sequence
  Left target
tgtgaggggttaaggctg

SEQ ID NO: 19 - DNA, 18 bp, Artificial Sequence
  Right target
tccccaccccttggtcca

SEQ ID NO: 20 - DNA, 18 bp, Artificial Sequence
  Left target
tttcggccaggctgaggc

SEQ ID NO: 21 - DNA, 18 bp, Artificial Sequence
  Right target
ttcccaggcctgcagttt

SEQ ID NO: 22 - DNA, 18 bp, Artificial Sequence
  Left target
tccgagcgtgggtctccg

SEQ ID NO: 23 - DNA, 18 bp, Artificial Sequence
  Right target
tagggggcgggcccggat

SEQ ID NO: 24 - DNA, 17 bp, Artificial Sequence
  Left target
tccagggcctggacagg

SEQ ID NO: 25 - DNA, 16 bp, Artificial Sequence
  Right target
tcggggtcctcctgtc

SEQ ID NO: 26 - DNA, 9159 bp, Artificial Sequence
  Left TALEN expression plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcaccatggactacaaagaccatgacggtgattataaagatcatgacatcgattacaaggatgacgatgacaagatggcccccaagaagaagaggaaggtgggcattcaccgcggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacagagacgattaaatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctccaatattggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgccaacaacaacggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaagcaatgggggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgccaacaacaacggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcatcccacgacggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgacacccgaacaggtggtcgccattgctaataataacggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttaggtccggcggcggagagggcagaggaagtcttctaacatgcggtgacgtggaggagaatcccggcccaatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgcctaaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgtcgacaggtgcttctcgatctgcatcctgggatcaaagcgatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 27 - DNA, 9191 bp, Artificial Sequence
  Right TALEN expression plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcaccatggactacaaagaccatgacggtgattataaagatcatgacatcgattacaaggatgacgatgacaagatggcccccaagaagaagaggaaggtgggcattcaccgcggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacagagacgattaaatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtcgaacattggagggaaacaagcattggagactgtccaacggctccttcccgtgttgtgtcaagcccacggtttgacgcctgcacaagtggtcgccatcgcctccaatattggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaagcaacatcggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaagcaacatcggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcatcccacgacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgacacccgaacaggtggtcgccattgctaataataacggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttaggtccggcggcggagagggcagaggaagtcttctaacatgcggtgacgtggaggagaatcccggcccaatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgtcgacaggtgcttctcgatctgcatcctgggatcaaagcgatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 28 - DNA, 23 bp, Artificial Sequence
  Primer
ggtgtgatcgtggatactaaagc

SEQ ID NO: 29 - DNA, 94 bp, Artificial Sequence
  Primer
tgggccgggattctcctccacgtcaccgcatgttagaagacttcctctgccctctccgccgccggacctaaagtttatctcgccgttattaaat

SEQ ID NO: 30 - DNA, 52 bp, Artificial Sequence
  Primer
atgcggtgacgtggaggagaatcccggcccaatgcccgccatgaagatcgag

SEQ ID NO: 31 - DNA, 50 bp, Artificial Sequence
  Primer
ctcaatggtgatggtgatgatgaccggtttaggcgaaggcgatgggggtc

SEQ ID NO: 32 - DNA, 21 bp, Artificial Sequence
  Primer
ctgacttcctctaaggttaat

SEQ ID NO: 33 - DNA, 20 bp, Artificial Sequence
  Primer
ggcaactagaaggcacagtc

SEQ ID NO: 34 - DNA, 20 bp, Artificial Sequence
  Primer
ccggcggattcccgagagaa

SEQ ID NO: 35 - DNA, 24 bp, Artificial Sequence
  Primer
cagctcgctcattgggccgggatt

SEQ ID NO: 36 - DNA, 24 bp, Artificial Sequence
  Primer
cccggcccaatgagcgagctgatt

SEQ ID NO: 37 - DNA, 27 bp, Artificial Sequence
  Primer
cccgaccggttaattaagcttgtgccc

SEQ ID NO: 38 - DNA, 21 bp, Artificial Sequence
  Primer
ctgacttcctctaaggttaat

SEQ ID NO: 39 - DNA, 20 bp, Artificial Sequence
  Primer
ggcaactagaaggcacagtc

SEQ ID NO: 40 - DNA, 6789 bp, Artificial Sequence
  Primer
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccatgagctccgccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcatggcccaacttgtttattgcagcttataatggttacaaataaagcaatagcatcacaaatttcacaaataaagcatttttttcactgcattctagttgtggtttgtccaaactcatcaatgtatcttatcatgtctggatctcctgcagataacttcgtatagcatacattatacgaagttatattaagggttccggatctcgaccagcttctgatggaattagaacttggcaaaacaatactgagaatgaagtgtatgtggaacagaggctgctgatctcgttcttcaggctatgaaactgacacatttggaaaccacagtacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcatcccggggatctgatatcatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtaagcttcagctgctcgagatctagatggatgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccacccttcccagctgctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaatttcgaggtcgagatcctaagcttggctggacgtaaactcctcttcagacctaataacttcgtatagcatacattatacgaagttatgtcgacgcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgggtacctcttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 41 - DNA, 50 bp, Artificial Sequence
  Primer
ggcctaggcgcgccatgagctccgccctcggtgtccccacaggatgaaac

SEQ ID NO: 42 - DNA, 50 bp, Artificial Sequence
  Primer
gcaataaacaagttgggccatgccgtgccccgggcactcagtcttccaac

SEQ ID NO: 43 - DNA, 140 bp, Artificial Sequence
  Single stranded oligo comprising left and right homology (mutant)
gggaggggccgggtccgcggccggcgaacggggctcgaagggtccttgtagccgggaatgctgctgctgctgctggggggatatcagaccatttctttctttcggccaggctgaggccctgacgtggatgggcaaactgc

SEQ ID NO: 44 - DNA, 4140 bp, Artificial Sequence
  Cas9
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 45 - DNA, 96 bp, Artificial Sequence
  gRNA14189 with scaffold sequence
tcgaagggtccttgtagccgttttagagctagaaatagcaagttaaaataaggctagtccgttatcaacttgaaaaagtggcaccgagtcggtgct

SEQ ID NO: 46 - DNA, 97 bp, Artificial Sequence
  gRNA14254 with scaffold sequence
gctgctgctgctgctgctgcgttttagagctagaaatagcaagttaaaataaggctagtccgttatcaacttgaaaaagtggcaccgagtcggtgct

SEQ ID NO: 47 - DNA, 1001 bp, Homo sapiens
tttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagc

SEQ ID NO: 48 - DNA, 421 bp, Artificial Sequence
  gRNA14189 with scaffold sequence and U6 promoter
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcgaagggtccttgtagccgttttagagctagaaatagcaagttaaaataaggctagtccgttatcaacttgaaaaagtggcaccgagtcggtgcttttttt

SEQ ID NO: 49 - DNA, 421 bp, Artificial Sequence
  gRNA14254 with scaffold sequence and U6 promoter
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgctgctgctgctgctgcgttttagagctagaaatagcaagttaaaataaggctagtccgttatcaacttgaaaaagtggcaccgagtcggtgcttttttt

SEQ ID NO: 50 - DNA, 19 bp, Artificial Sequence
  gRNA14189 without scaffold sequence
tcgaagggtccttgtagcc

SEQ ID NO: 51 - DNA, 20 bp, Artificial Sequence
  gRNA14254 without scaffold sequence
gctgctgctgctgctgctgc

SEQ ID NO: 52 - DNA, 3397 bp, Artificial Sequence
  Donor molecule comprising left and right homology
ctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccggccatggcccaacttgtttattgcagcttataatggttacaaataaagcaatagcatcacaaatttcacaaataaagcatttttttcactgcattctagttgtggtttgtccaaactcatcaatgtatcttatcatgtctggatctctcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtaagcttcagctgctcgagatctagatggatgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccacccttcccagctgctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaatttcgaggtcgagatcctaagcttggctggacgtaaactcctcttcagacctactgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaagctcagtctcggggcggaggcttcgcgcccttagtc

SEQ ID NO: 53 - DNA, 140 bp, Artificial Sequence
  Single stranded oligo comprising left and right homology (wild type)
gggaggggccgggtccgcggccggcgaacggggctcgaagggtccttgtagccgggaatgctgctgctgctgctggggggatcacagaccatttctttctttcggccaggctgaggccctgacgtggatgggcaaactgc

SEQ ID NO: 54 - DNA, 60 bp, Artificial Sequence
  left homology
gggaggggccgggtccgcggccggcgaacggggctcgaagggtccttgtagccgggaatg

SEQ ID NO: 55 - DNA, 65 bp, Artificial Sequence
  Right homology (mutant)
gggggatatcagaccatttctttctttcggccaggctgaggccctgacgtggatgggcaaactgc

SEQ ID NO: 56 - DNA, 65 bp, Artificial Sequence
  Right homology (wild type)
gggggatcacagaccatttctttctttcggccaggctgaggccctgacgtggatgggcaaactgc

SEQ ID NO: 57 - DNA, 1026 bp, Artificial Sequence
  Left homology
ctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgg

SEQ ID NO: 58 - DNA, 1029 bp, Artificial Sequence
  Right homology
ctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaagctcagtctcggggcggaggcttcgcgcccttagtc

SEQ ID NO: 59 - DNA, 10474 bp, Artificial Sequence
  Cas9 expression plasmid
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgaaagggttcgatccctaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 60 - DNA, 6399 bp, Artificial Sequence
  Donor molecule
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccggagctcccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcctgcagataacttcgtatagcatacattatacgaagttatattaagggttccggatctcgaccagcttctgatggaattagaacttggcaaaacaatactgagaatgaagtgtatgtggaacagaggctgctgatctcgttcttcaggctatgaaactgacacatttggaaaccacagtacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcatcccggggatctgatatcatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtaagcttcagctgctcgagatctagatggatgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccacccttcccagctgctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaatttcgaggtcgagatcctaagcttggctggacgtaaactcctcttcagacctaataacttcgtatagcatacattatacgaagttatgtcgacctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaagctcagtctcggggcggaggcttcgcgcccttagtcggtacctcttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 61 - DNA, 5250 bp, Artificial Sequence
  Guide RNA 14189 expression plasmid
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgccctttaatgccaactttgtacaagaaagctgggtctagaaaaaaagcaccgactcggtgccactttttcaagttgataacggactagccttattttaacttgctatttctagctctaaaacggctacaaggacccttcgacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttggtaccggatccagtcgactgaattggttcctttaaagcctgcttttttgtacaaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgaggagggccaccatggccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 62 - DNA, 5250 bp, Artificial Sequence
  Guide RNA 14254 expression plasmid
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgccctttaatgccaactttgtacaagaaagctgggtctagaaaaaaagcaccgactcggtgccactttttcaagttgataacggactagccttattttaacttgctatttctagctctaaaacgcagcagcagcagcagcagcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttggtaccggatccagtcgactgaattggttcctttaaagcctgcttttttgtacaaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgaggagggccaccatggccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 63 - DNA, 24 bp, Artificial Sequence
  primer
ccctcctaggccgccatgcattag

SEQ ID NO: 64 - DNA, 30 bp, Artificial Sequence
  primer
cccgttcggtccgcgccttaagatacattg

SEQ ID NO: 65 - DNA, 19 bp, Artificial Sequence
  primer
cctctgcctctgagctatt

SEQ ID NO: 66 - DNA, 21 bp, Artificial Sequence
  primer
gataccgtaaagcacgaggaa

SEQ ID NO: 67 - DNA, 26 bp, Artificial Sequence
  primer
cccgtctgtcgacctgctgctggggg

SEQ ID NO: 68 - DNA, 26 bp, Artificial Sequence
  primer
cccgtctgtcgacctgctgctggggg

SEQ ID NO: 69 - DNA, 19 bp, Artificial Sequence
  primer
ctaagcttggctggacgta

SEQ ID NO: 70 - DNA, 19 bp, Artificial Sequence
  primer
cctatggaaaaacgccagc

SEQ ID NO: 71 - DNA, 24 bp, Artificial Sequence
  primer
ccttggcgcgcctccctggctcct

SEQ ID NO: 72 - DNA, 23 bp, Artificial Sequence
  primer
ccctgagctccggctacaaggac

SEQ ID NO: 73 - DNA, 20 bp, Artificial Sequence
  primer
gatgtgctgcaaggcgatta

SEQ ID NO: 74 - DNA, 22 bp, Artificial Sequence
  primer
ccacaactagaatgcagtgaaa

SEQ ID NO: 75 - DNA, 60 bp, Artificial Sequence
  Cr14189_Insert_F annealing oligo
tttcttggctttatatatcttgtggaaaggacgaaacaccgtcgaagggtccttgtagcc

SEQ ID NO: 76 - DNA, 60 bp, Artificial Sequence
  Cr14189_Insert_R annealing oligo
gactagccttattttaacttgctatttctagctctaaaacggctacaaggacccttcgac

SEQ ID NO: 77 - DNA, 29 bp, Artificial Sequence
  primer
ccctggccaccatggccgccatgcattag

SEQ ID NO: 78 - DNA, 30 bp, Artificial Sequence
  primer
ccctcacgaagtgcgccttaagatacattg

SEQ ID NO: 79 - DNA, 30 bp, Artificial Sequence; identical to SEQ ID NO: 78
  primer

SEQ ID NO: 80 - DNA, 20 bp, Artificial Sequence
  primer
ggtatctgcgctctgctgaa

SEQ ID NO: 81 - DNA, 20 bp, Artificial Sequence
  primer
gtgtacggtgggaggtctat

SEQ ID NO: 82 - DNA, 18 bp, Artificial Sequence
  primer
caggaaacagctatgacc

SEQ ID NO: 83 - DNA, 60 bp, Artificial Sequence
  Cr 14254 _Insert_F annealing oligo
tttcttggctttatatatcttgtggaaaggacgaaacaccgctgctgctgctgctgctgc

SEQ ID NO: 84 - DNA, 60 bp, Artificial Sequence
  Cr 14254 _Insert_R annealing oligo
gactagccttattttaacttgctatttctagctctaaaacgcagcagcagcagcagcagc

SEQ ID NO: 85 - DNA, 29 bp, Artificial Sequence
  primer
ccctggccaccatggccgccatgcattag

SEQ ID NO: 86 - DNA, 30 bp, Artificial Sequence; identical to SEQ ID NO: 78
  primer

SEQ ID NO: 87 - DNA, 305 bp, Artificial Sequence
  fragment guide RNA cloning vector
aggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 88 - DNA, 178 bp, Artificial Sequence
  fragment guide RNA cloning vector
gttttagagctagaaatagcaagttaaaataaggctagtccgttatcaacttgaaaaagtggcaccgagtcggtgctttttttaagcttgggccgctcgaggtacctctctacatatgacatgtgagcaaaaggccagcaaaaggccaggaaccgtaaaaaggccgcgttgctggcgt

SEQ ID NO: 89 - DNA, 23 bp, Artificial Sequence
  target sequence + PAM
gctcgaagggtccttgtagccgg

SEQ ID NO: 90 - DNA, 23 bp, Artificial Sequence
  target sequence  + PAM
gccggcgaacggggctcgaaggg

SEQ ID NO: 91 - DNA, 23 bp, Artificial Sequence
  target sequence + PAM
gggtccgcggccggcgaacgggg

SEQ ID NO: 92 - DNA, 23 bp, Artificial Sequence
  target sequence + PAM
gccaggctgaggccctgacgtgg

SEQ ID NO: 93 - DNA, 23 bp, Artificial Sequence
  target sequence + PAM
gctgaggccctgacgtggatggg

SEQ ID NO: 94 - DNA, 23 bp, Artificial Sequence
  target sequence + PAM
gcagtttgcccatccacgtcagg

SEQ ID NO: 95 - DNA, 20 bp, Artificial Sequence
  target sequence + PAM
ggcgaacggggctcgaaggg

SEQ ID NO: 96 - DNA, 21 bp, Artificial Sequence
  target sequence + PAM
gtccgcggccggcgaacgggg

SEQ ID NO: 97 - DNA, 20 bp, Artificial Sequence
  target sequence + PAM
gaggccctgacgtggatggg

SEQ ID NO: 98 - DNA, 20 bp, Artificial Sequence
  target sequence + PAM
gtttgcccatccacgtcagg

SEQ ID NO: 99 - DNA, 23 bp, Artificial Sequence
  target sequence + PAM
gttaaggctgggaggcgggaggg

SEQ ID NO: 100 - DNA, 23 bp, Artificial Sequence
  target sequence + PAM
ggtcctcctgtcacagggcctgg

SEQ ID NO: 101 - DNA, 23 bp, Artificial Sequence
  target sequence + PAM
gggcctggacaggggctgccagg

SEQ ID NO: 102 - DNA, 21 bp, Artificial Sequence
  target sequence  + PAM
gcatctcacctctatgggagg

SEQ ID NO: 103 - DNA, 23 bp, Artificial Sequence
  target sequence + PAM
ggcatctcacctctatgggaggg

SEQ ID NO: 104 - DNA, 20 bp, Artificial Sequence
  pFYF1884 target seqeunce 5′ CTG repeat
gctcgaagggtccttgtagc

SEQ ID NO: 105 - DNA, 20 bp, Artificial Sequence
  pFYF1885 target 5′ CTG repeat
gccggcgaacggggctcgaa

SEQ ID NO: 106 - DNA, 20 bp, Artificial Sequence
  pFYF1886 target 5′ CTG repeat
gggtccgcggccggcgaacg

SEQ ID NO: 107 - DNA, 20 bp, Artificial Sequence
  pFYF1887 target 3′ CTG repeat
gccaggctgaggccctgacg

SEQ ID NO: 108 - DNA, 20 bp, Artificial Sequence
  pFYF1888 target 3′ CTG repeat
gctgaggccctgacgtggat

SEQ ID NO: 109 - DNA, 20 bp, Artificial Sequence
  pFYF1889 target 3′ CTG repeat
gcagtttgcccatccacgtc

SEQ ID NO: 110 - DNA, 17 bp, Artificial Sequence
  pFYF1890 target 5′ CTG repeat
ggcgaacggggctcgaa

SEQ ID NO: 111 - DNA, 18 bp, Artificial Sequence
  pFYF1891 target 5′ CTG repeat
gtccgcggccggcgaacg

SEQ ID NO: 112 - DNA, 17 bp, Artificial Sequence
  pFYF1892 target 3′ CTG repeat
gaggccctgacgtggat

SEQ ID NO: 113 - DNA, 17 bp, Artificial Sequence
  pFYF1881 target 3′ CTG repeat
gtttgcccatccacgtc

SEQ ID NO: 114 - DNA, 20 bp, Artificial Sequence
  pFYF1896 target SP1
gttaaggctgggaggcggga

SEQ ID NO: 115 - DNA, 20 bp, Artificial Sequence
  pFYF1899 target AP2
ggtcctcctgtcacagggcc

SEQ ID NO: 116 - DNA, 20 bp, Artificial Sequence
  pFYF1902 target AP2
gggcctggacaggggctgcc

SEQ ID NO: 117 - DNA, 18 bp, Artificial Sequence
  pFYF1905 target ATG
gcatctcacctctatggg

SEQ ID NO: 118 - DNA, 20 bp, Artificial Sequence
  pFYF1908 target ATG
ggcatctcacctctatggga

SEQ ID NO: 119 - DNA, 34 bp, Artificial Sequence
  PCR primer
atcagctacgtacggactggatccggtaccaagg

SEQ ID NO: 120 - DNA, 39 bp, Artificial Sequence
  PCR primer
gtcgcagctaactagtcccaagcttaaaaaaagcaccga

SEQ ID NO: 121 - DNA, 14 bp, Artificial Sequence
  generic tale structure
tttattccctgacc

SEQ ID NO: 122 - protein, 34 aa, Artificial Sequence
  generic tale structure
LTPEQVVAIASNGGGKQALETVQRLLPVLCQAHG

SEQ ID NO: 123 - DNA, 144 bp, Artificial Sequence
  DMPK fragment
gggagggaggggccgggtccgcggccggcgaacggggctcgaagggtccttgtagccgggaatgctgctgctgctgctggggggatatcagaccatttctttctttcggccaggctgaggccctgacgtggatgggcaaactgc

SEQ ID NO: 124 - DNA, 36 bp, Artificial Sequence
  DMPK gene 3′ region ctg repeat
gccaggctgaggccctgacgtggatgggcaaactgc

SEQ ID NO: 125 - DNA, 42 bp, Artificial Sequence
  DMPK gene 3′ region ctg repeat
gggtccgcggccggcgaacggggctcgaagggtccttgtagc

SEQ ID NO: 126 - RNA, 80 nt, Artificial Sequence
  scaffold part of sgRNA
guuuuagagcuagaaauagcaaguuaaaauaaggcuaguccguuaucaacuugaaaaaguggcaccgagucggugcuuuu

(19) United States
(12) Patent Application Publication
(10) Pub. No.: US 2017/0088819 A1
(43) Pub. Date: Mar. 30, 2017

(54) GENETIC CORRECTION OF MYOTONIC DYSTROPHY TYPE 1

(71) Applicants: VRIJE UNIVERSITEIT BRUSSEL, Brussel (BE); THE GENERAL HOSPITAL CORPORATION, Boston, MA (US)

(72) Inventors: Thierry VANDENDRIESSCHE, Bierbeek (BE); Marinee CHUAH, Bierbeek (BE); J. Keith JOUNG, Winchester, MA (US); Yanfang FU, Malden, MA (US); Deepak REYON, Cambridge, MA (US)

(21) Appl. No.: 15/311,579

(22) PCT Filed: May 18, 2015
(86) PCT No.: PCT/EP2015/060922

§ 371 Date: Nov. 16, 2016

(30) Foreign Application Priority Data
May 16, 2014 (EP) 14168688.1
May 16, 2014 (EP) 14168708.7

(51) Int. Cl. C12N 5/074; C12N 9/12; A61K 35/34; C12N 15/90

(52) U.S. Cl. CPC C12N 5/0696; C12N 15/907; C12N 9/1205; A61K 35/34; C12N 2800/80; A61K 48/00

(57) ABSTRACT

The invention relates to polynucleotides suitable for reducing or eliminating the expression of expanded repeat RNA (CUGexp) of the dystrophy myotonic-protein kinase (DMPK) gene in a cell of a DM-1 patient. The polynucleotides are a combination of a polynucleotide for a site specific nuclease targeting the dystrophy myotonic-protein kinase (DMPK) gene locus, and a donor polynucleotide having 5′ and 3′ regions which are homologous with the sequence of DMPK gene which flank the target site of the nuclease. The invention further relate to in vivo and in vitro methods to reduce or eliminate CTG repeats in the DMPK gene. The invention further relates to the medical use of polynucleotides and cells for treating DM-1 patient.

### FIELD OF THE INVENTION

Provided herein are compositions and methods for the treatment of myotonic dystrophy type 1 (DM1). The present invention in particular relates to compositions and methods involving genetic correction of DM1-derived induced pluripotent stem cells (iPS) or its differentiated progeny, in particular muscle-like or myogenic cells, as well as in vitro and in vivo use of DM1-derived iPS or its differentiated progeny, in particular muscle-like or myogenic cells.

### BACKGROUND OF THE INVENTION

Myotonic dystrophy type 1 (DM1) is a dominantly inherited neurodegenerative disorder that afflicts 1 in 8000 individuals. There is currently no cure or effective treatment available. DM1 is not caused by expression of a mutant protein, but instead is due to expression of a pathogenic RNA. Indeed, expression of the mutated DMPK gene gives rise to an expanded repeat RNA (CUGexp) that is directly toxic to cells by interfering with splicing, expression and function of multiple target genes. The mutant RNA is retained in the nucleus, forming ribonuclear inclusions in affected tissue. Targeting the pathogenic CUGexp or preventing its expression represents one of the therapeutic strategies to treat DM1. It has been shown recently that inhibition of the CUGexp could effectively inhibit the spliceopathy in myoblasts obtained from DM1 patients (Francois et al. (2011) Nat. Struct. Mol. Biol. 18, 85-87).

Though cellular models may seem relatively straightforward to set up for screening therapeutic molecules, however, two major difficulties hamper the use of primary human cardiomyocytes or skeletal muscle progenitors cultures: the accessibility and availability of muscle biopsies from patients affected with DM1, and the limited proliferative capacity of adult human primary cells such as myoblasts. It is particularly challenging to obtain large biopsies from patients and consequently to obtain a sufficient number of cells for extensive ex vivo studies. To overcome these limitations, induced pluripotent stem cells (iPS) can be used instead.

Moreover, it is not known whether neutralization of the toxic effects of the DM1 CUGexp RNA would also restore the potentially lethal severe cardiac abnormalities in DM1. Hence, it is also necessary to evaluate whether phenotypic correction of cardiac defects can be achieved, that typically contribute to significant mortality and morbidity in patients suffering from DM1.

There is currently no cure or effective treatment available for myotonic dystrophy and fragile X tremor ataxia syndrome (FXTAS). Furthermore, there exists a need for tools in order to study and treat RNA-dominant genetic disorders such as myotonic dystrophy. A pilot experiment was published by Rodriguez et al (2014) Mol. ther. 22S1, pS94 11. The present invention aims at the treatment of RNA-dominant genetic disorders, as well as the consolidation of a novel platform technology to develop and validate novel therapeutic approaches for such disorders and to allow for a better understanding of the underlying biological defects.

### SUMMARY OF THE INVENTION

The present inventors have found that it is virtually impossible to expand DM1 patient derived precursor cells, such as myogenic cells (e.g. myoblasts or mesoangioblasts) or neuronal cells. While such cells may be isolated and to a certain extent propagated in vitro, after a few passages, the cells lose their proliferative capacity, and eventually die out. Without wishing to be bound by theory, it seems plausible that the toxic accumulation of defective DMPK mRNA may contribute to this effect. This undermines for instance the use of DM1 primary cells (such as muscle derived cells, be it myoblast or mesoangioblasts, or neuronal cells) for drug screening, disease investigation, and regenerative medicine. The present inventors have however, found that it is possible to derive induced pluripotent stem cells (iPS) from cells originating from subjects afflicted with myotonic dystrophy type 1 (DM1). More surprising, these DM1 patient derived iPS cells are capable of being differentiated into myogenic precursor cells, such as cardiomyogenic cells, myoblast- or mesoangioblast-like cells, or alternatively or neuronal or neurogenic cells. Importantly, and unexpectedly, both the DM1 derived iPS as well as the myogenic precursor cells derived therefrom display a DM1 specific phenotype, i.e. nuclear foci, which are characteristic for nuclear RNA accumulation associated with DM1. The iPS-derived precursors, such as the myogenic or neurogenic precursors, provide for an unprecedented opportunity to replicate both normal and pathologic human tissue, such as muscle or nerve tissue, formation in vitro, that impacts on disease investigation, drug development and regenerative medicine. The availability of such platform overcomes some of the bottlenecks intrinsic to the use of patient-derived primary cells, such as myoblasts or mesoangioblasts, but also neuronal or neurogenic cells, which have a much more restricted life-span. An additional important advantage of the DM1 derived iPS, in particular the nuclear foci phenotype, is that differentiation of cellular commitment, such as myogenic or neurogenic commitment, is not necessary if drug screening purposes can be done from the DM1 undifferentiated iPS cells. In addition, the present inventors have found that DM1-derived iPS, as well as the precursors derived therefrom, such as the myogenic or neurogenic precursors derived therefrom, are less fragile than primary DM1-derived cells, such as primary myogenic or neurogenic cells, and can advantageously be subjected to gene transfer, with minimal loss of cell death and proliferative capacity, in contrast to primary DM1-derived cells, which experience a vast amount of cell death and the surviving cells often even fail to grow anymore after transfection. In view of the limited proliferative capacity of primary DM1-derived cells, their possible application for transplantation is severely limited. In contrast, the DM1-derived iPS and their progeny, such as myogenic or neurogenic progeny, provide for a more robust cell system platform, which, in view of their continued proliferative capacity, may be readily used not only for a variety of in vitro assays, but also for transplantation, for instance after in vitro and in vivo gene correction.

Hereto, the present invention is in particular captured by any one or any combination of one or more of the below numbered statements of aspects and embodiments (i) to (xxxii).

(i) A combination of:
a. a polynucleotide for a site specific nuclease targeting the dystrophy myotonic-protein kinase (DMPK) gene locus, and
b. a donor polynucleotide having 5′ and 3′ regions which are homologous with the sequence of DMPK gene which flank the target site of the nuclease defined in a), the combination of polynucleotides being suitable for reducing or eliminating the expression of expanded repeat RNA (CUGexp) of the dystrophy myotonic-protein kinase (DMPK) gene in a cell of a DM-1 patient.
(ii) The combination of polynucleotides according to statement (i), wherein said polynucleotide for said site specific nuclease is a clustered regulatory interspaced short palindromic repeat (CRISPR) guide RNA of a Cas-based RNA-guided DNA endonuclease.
(iii) The combination of polynucleotides according to statement (i) or (ii), further comprising a polynucleotide sequence encoding a Cas9 endonuclease.
(iv) The combination of polynucleotides according to any one of statements (i) to (iii), wherein the CRISPR guide RNA and/or the Cas-based RNA-guided DNA endonuclease are comprised within a lentiviral vector.
(v) The combination of polynucleotides according to any one of statements (i) to (iv), wherein the CRISPR guide RNA is capable of specifically binding to the junction between the DMPK gene sequence and the expanded CTG trinucleotide repeat; capable of binding to the SP1 binding site of the DMPK promoter; capable of binding to the AP-2 binding site of the DMPK promoter; or capable of binding to the start codon of the DMPK gene.
(vi) The combination of polynucleotides according to any one of statements (i) to (v), wherein the CRISPR guide RNA is capable of specifically binding to the SP1 or AP-2 binding site of the DMPK promoter.
(vii) The combination of polynucleotides according to any one of statements (i) to (vi), wherein the CRISPR guide RNA is capable of specifically binding at the junction between the DMPK gene sequence and the expanded CTG trinucleotide repeat.
(viii) The combination of polynucleotides according to any one of statements (i) to (vii), wherein the target sequence of the guide RNA does not overlap with part of the CTG trinucleotide repeat.
(ix) The combination of polynucleotides according to any one of statements (i) to (viii), comprising two CRISPR guide RNA molecules, the first one capable of specifically binding at the 5′ junction with the expanded CTG trinucleotide repeat and/or the second one capable of specifically binding at the 3′ junction of the expanded CTG trinucleotide repeat. The distance between the CTG repeat and the target sequence of the guideRNA of a CRISPR can in certain embodiments range from 1, 5, 10, 15, 20, 25, 30, 40 or 50 nucleotides.
(x) The combination of polynucleotides to any one of statements (i) to (viii), comprising two CRISPR guide RNA molecules, the first one capable of specifically binding upstream of the 5′ end of expanded CTG trinucleotide repeat, and/or the second one capable of specifically binding downstream of the 3′ end of the expanded CTG trinucleotide repeat, wherein the target sequence of the guide RNA of one or both guide RNAs does not overlap with part of the CTG trinucleotide repeat.
(xi) The combination of polynucleotides according to any one of statements (i) to (x), wherein the target sequence of the guide RNA is between xvii and 20 nucleotides.
(xii) The combination of polynucleotides according to any one of statement (i) to (xi), wherein the target sequence of CRISPR guide RNA sequence has a sequence selected from the group consisting of SEQ ID NO: 50, 51 and 104 to 118, wherein T may be replaced by U.
(xiii) The combination of polynucleotides according to statement (i), wherein said polynucleotide for said site specific nuclease encodes for a designer transcription activator-like effector nuclease (dTALEN).
(xiv) The combination according to statement (xiii), wherein the sequence coding for the DNA binding part of the dTALEN is depicted by SEQ ID NO:1 or SEQ ID NO:2.
(xv) The combination of polynucleotides according to any one of statements (i) to (xiv), wherein the donor molecule comprises no protein-encoding sequence inbetween the 5′ and 3′ regions which are homologous with the sequence of DMPK gene which flank the target site.
(xvi) The combination of polynucleotides according to any one of statements (i) to (xv), wherein the 5′ and/or 3′ regions of the donor which are homologous with the sequence of DMPK have a length of about 800 to 1000 nucleotides.
(xvii) The combination of polynucleotides according to any one of statements (i) to (xvi), wherein the donor comprises at the 5′ end a region which binds the 5′ of the CTG repeat of the DMPK gene and comprises at the 3′ end a region which binds the 3′ of the CTG repeat of the DMPK gene and which comprises in between the two regions 5 to 30 CTG repeats.
(xviii) An in vitro method of reducing or elimination the expression of expanded repeat RNA (CUGexp) of the dystrophy myotonic-protein kinase DMPK gene in cells originating from a subject having myotonic dystrophy type 1 (DM1), comprising the steps of:
a. introducing in said cells a polynucleotide for a site specific nuclease targeting the dystrophy myotonic-protein kinase (DMPK) gene,
b. introducing in said cells a donor polynucleotide having 5′ and 3′ regions which are homologous with the sequence of the DMPK gene which flank the target site of the polynucleotide defined in a).
(xix) The method according to statement (xviii), wherein said polynucleotide producing a site specific nuclease comprises a polynucleotide for expression of a Cas based RNA-guided DNA endonuclease nuclease and comprises a polynucleotide for the translation of a clustered regulatory interspaced short palindromic repeat (CRISPR) guide RNA for said endonuclease.
(xx) The method according to statement (xviii) or (xix), wherein said nucleotide for expression of said nuclease, and/or said nucleotide for translation of guide RNA is lentiviral vector.
(xxi) The method according to any one of statements (xviii) to (xx), where the cells are iPSC or progenitor cells derived thereof.
(xxii) The method according to any one of statements (xviii) to (xxi), wherein the subject is a mouse model for DM-1.
(xxiii) The combination of polynucleotides according to any one of statements (i) to (xvii), for use in treating DM-1.
(xxiv) A population of cells, obtained by a method according to any one of statements (xviii) to (xxii), for use in treating DM-1.
(xxv) In vitro use of the combination of polynucleotides according to any one of statements (i) to (xvii), for reducing or eliminating the expression of expanded repeat RNA (CUGexp) of the dystrophy myotonic-protein kinase (DMPK) gene in a cell originating from a DM-1 patient.
(xxvi) The use according to statement (xxv), wherein the patient is a mouse model for DM-1.
(xxvii) A method of reducing the expression of expanded repeat RNA (CUGexp) of the DMPK gene in a subject having myotonic dystrophy type 1 (DM1), comprising the steps of administering to said subject:
a. a polynucleotide for a site specific nuclease targeting the dystrophy myotonic-protein kinase (DMPK) gene,
b. a donor polynucleotide having 5′ and 3′ regions which are homologous with the sequence of DMPK gene which flank the target site of the polynucleotide defined in a).
(xxviii) The method according to statement (xxvii), wherein the subject is a mouse model for DM-1.
(xxix) An method of reducing the expression of expanded repeat RNA (CUGexp) of the DMPK gene in a subject having myotonic dystrophy type 1 (DM1), comprising the steps of:
a. isolating cells from said subject and converting said cells to iPS cells,
b. subjecting these cells to a method as defined in any one of statements xviii to xxii,
c. introducing said cells obtained in step, optionally after differentiation into muscle precursor or progenitor cells, to said subject.
(xxx) The method according to statement (xxix), wherein the subject is a mouse model for Dm-1.
(xxxi) A polynucleotide for a CRISPR/cas comprising a target sequence consisting of a sequence selected from the group consisting of SEQ ID NO: 50, 51 and 104 to 118, or the complement or the reverse complement of said polynucleotide, wherein T may be replaced by U.
(xxxii) A vector comprising a polynucleotide according to statement xxxi.
The appended claims are also explicitly included in the description.

### BRIEF DESCRIPTION OF THE FIGURES

FIG. 1: Phase contrast images of L22, L23 & L81 DM1-iPS clones showing compact, undifferentiated colonies with intact border on feeder free vitronectin-coated dishes.

FIG. 2: Pluripotency marker expression: Representative pictures of hiPS cell colonies for DM1 clone L22, L23 and L81 stained for pluripotent markers alkaline phosphatase (AP), SSEA 3, hTRA 1-60, hOCT4, and SSEA 4. Nuclei are DAPI stained

FIG. 3: (a) H&E staining of the histological sections of the iPS derived teratoma generated from the three DM1 iPS clones (L22, L23 and L81) injected in immuno—compromised CB17—SCID mice; the teratoma showed the presence of tissues derived from the three germlayers, i.e. endoderm, mesoderm and ectoderm. (b) The mice developed teratomas in 6 to 8 weeks.

FIG. 4: Array Comparative Genomic Hybridization (aCGH) on DM1-iPS.

FIG. 5: FISH in DM1 iPS cells for the detection of nuclear foci. DM1 myoblasts were used as positive control whereas iPS cells from a healthy donor were used as negative control.

FIG. 6: Real time PCR data of the SK3 gene expression from the three DM1 iPS clones and the control iPS cells which is designated as control. The control level is indicated as 1 and the fold up-regulation of the SK3 expression of the DM1 clones were indicated on top of each black bar. * indicate statistical significance with p<0.05 and ** indicate p<0.001.

FIG. 7: Schematic flowchart of the steps involved in the differentiation of iPS cells into muscle cells. (Tedesco et al. (2012) Sci Transl Med 4, 140ra89.)

FIG. 8: Phase contrast images of HIDEMs for DM1 clones L81 and L23 from both feeder free and feeder condition & L22 HIDEMs from DM1-iPS clones from feeder condition. Control HIDEMs were from iPS from healthy donor. The images are at 10× magnification.

FIG. 9: Surface marker expression in HIDEMs: HIDEMs were stained with fluorochrome conjugated primary antibodies against CD13, CD31, CD44, CD49b, CD45, CD146, SSEA4 and AP.

FIG. 10: Lamin AC marker expression in HIDEMs: Representative pictures of HIDEMs derived from DM1 iPS clone L23 and L81 stained for nuclear marker Lamin AC) counter stained with nuclear stain DAPI. The HIDEMs from feeder free iPS cultures were taken as an internal control. Control HIDEMs are from wild type iPS cells.

FIG. 11: Alkaline Phosphatase staining of HIDEMs for DM1 clones L81 and L23 from both feeder free and feeder condition. Control HIDEMs were from iPS from healthy donor. The images are at 10× magnification. The cells were counter stained with DAPI nuclear stain.

FIG. 12: Pluripotency marker expression: Representative pictures of HIDEMs derived from DM1 iPS clone L23 and L81 stained for pluripotent markers hOCT4, and SOX2. DM1 iPS cells were used as positive controls. Nuclear DAPI staining is shown.

FIG. 13: MyHC staining of the differentiated cells (myotube-like and myocyte-like phenotype). In the representative pictures, large myotubes with multiple nuclei can be seen.

FIG. 14: Schematic overview of targeting of the DMPK gene containing an expanded CTG repeat with a donor molecule using dTALEN.

FIG. 15: Plasmid maps of vectors comprising the donor molecule (A), (B), left dTALEN 1755 (C), and right dTALEN 1756 (D).

FIG. 16: Microscopic pictures of nucleofected L22 iPS cells 4 days post sorting.

FIG. 17: Microscopic pictures of nucleofected L22 iPS cells 4 days post sorting.

FIG. 18: Microscopic of the nucleofected L22 iPS cells pictures after the indicated days of puromycin selection.

FIG. 19: Results of RNA foci staining of L22 iPS targeted by TALEN along with donor molecule.

FIG. 20: Nuclear Foci staining of dTALEN nucleofected and sorted iPS cells. The appended table indicates the transfected constructs and the detection of RNA foci.

FIG. 21: Generic TALE structure and TALE code (taken from http://www.genome-engineering.org/taleffectors/).

FIG. 22: Schematic overview of a double TALEN pair approach for deletion of the DMPK expanded CTG repeat.

FIG. 23: Schematic overview of a single TALEN pair approach for disruption of the DMPK promoter.

FIG. 24: Schematic overview of replacement of the DMPK expanded CTG repeat with a single stranded (SS) oligo using CRISPR.

FIG. 25: Plasmid maps of vectors comprising the ssOligo (A), Cas9-BFP (B), gRNA CR14189 (C), and gRNA CR14254 (D).

FIG. 26: Nuclear Foci staining of CRISPR/Cas transfected and sorted HIDEMs cells.

FIG. 27: Nuclear Foci staining of CRISPR/Cas transfected and sorted HIDEMs cells (zoomed in on the nucleus). The appended table indicates the transfected constructs and the detection of RNA foci.

FIG. 28: Schematic overview of replacement of the DMPK expanded CTG repeat with a donor molecule using CRISPR.

FIG. 29: Plasmid maps of vectors comprising a donor molecule.

FIG. 30: Cell survival of CRISPR/Cas targeted cells with donor molecule, after indicated days of puromycin selection.

FIG. 31: Schematic overview of a double CRISPR guide RNA approach for deletion of the DMPK expanded CTG repeat.

FIG. 32: Schematic overview of a single CRISPR guide RNA approach for disruption of the DMPK promoter.

FIG. 33: Schematic overview of the complex between genomic DNA, guide RNA (target sequence and scaffold sequence) and Cas9 nuclease

FIG. 34: Reduction in nuclear foci using CRISPR/Cas with and without donor (see example 6)

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); Innis et al., PCR Protocols: A Guide to Methods and Applications, Academic Press: San Diego, 1990. General principles of microbiology are set forth, for example, in Davis, B. D. et al., Microbiology, 3rd edition, Harper & Row, publishers, Philadelphia, Pa. (1980).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

In an aspect, the present invention relates to induced pluripotent stem cells (iPS) derived from cells originating from a subject having myotonic dystrophy type 1 (DM1).

The term “myotonic dystrophy” has its meaning as is known in the art. By means of further guidance, this term generally refers to a chronic, slowly progressing, highly variable, inherited multisystemic disease characterized by wasting of the muscles (muscular dystrophy), cataracts, heart conduction defects, endocrine changes, and myotonia. Two types of myotonic dystrophy exist. Table 1 illustrates the differences between the myotonic dystrophy of type 1 and type 2. Myotonic dystrophy type 1 (DM1), also called Steinert disease, has a severe congenital form and a milder childhood-onset form. Myotonic dystrophy type 2 (DM2), also called proximal myotonic myopathy (PROMM) or adult-onset form, is rarer than DM1 and generally manifests with milder signs and symptoms. Myotonic dystrophy can occur in patients of any age. Both forms of the disease display an autosomal dominant pattern of inheritance.

TABLE 1

| Type | Gene | Repeat | Anticipation | Severity |
|---|---|---|---|---|
| DM1 | DMPK | CTG | Yes | Moderate-severe |
| DM2 | ZNF9 | CCTG | Minimal/none | Mild-moderate |

The term “myotonic dystrophy type 1” or “DM1” generally refers to a rare hereditary disorder of the neuromuscular and locomotor system caused by the expansion of the cytosine-thymine-guanine (CTG) triplet repeat located in the 3′-untranslated region (3′-UTR) of the dystrophy myotonic-protein kinase (DMPK) gene.

In DM1 patients, the skeletal muscle is severely affected with progressive muscle wasting and myotonia being two of the major clinical manifestations of this disease together with a progressive decline in maximal force production which represents one of the most disabling aspects of the disease. Moreover, cardiac arrhythmias and sudden death are a major cause of mortality in DM1 patients, even in young patients with limited muscle problems.

In DM1, RNA that is transcribed from DNA containing non-coding expansions is causative of disease pathogenesis. The expanded allele is transcribed to produce RNA containing expanded CUG repeats (CUGexp) that becomes stuck in nuclear foci, precluding its export to the cytoplasm for translation into DMPK protein. Although loss of DMPK contributes to the disease, toxicity of CUGexp RNA plays the major role.

CUGexp RNA folds into an imperfect hairpin structure that resembles the natural binding site for the protein muscleblind-like 1 (MBNL1). MBNL is consequently sequestered by the RNA, not only resulting in loss of its normal function in RNA splicing, but enhancing formation of foci that trap CUGexp RNA in the nucleus.

Another component of pathogenicity is aberrant activation of protein kinase C, which leads to increased activity of a second splicing regulator, CUG-binding protein 1 (CUGBP1). Both MBNL1 and CUGBP1 coordinately regulate the alternative splicing of pre-mRNA during development. CUGexp RNA disrupts this program, resulting in the aberrant expression of embryonic splicing patterns in adult tissues. One of the best-characterized misregulated splicing events in DM1 is of the RNA encoding the muscle-specific chloride channel (CLCN1). The altered splicing of CLCN1 results in the loss of this channel in DM1 patients. This results in skeletal muscle hyperexcitability, causing the myotonia for which the disease is named. Recently, the misregulation of BIN1 was also associated to the progressive weakness observed in muscle of the DM1 patients. However the role of many of the other mis-splicing events in the DM1 physiopathology is not yet fully understood. Finally, an additional mechanism related to RNA toxicity resulting from the activation of a transcription factor (i.e. Nkx 2.5) or the deregulation of miR1 biogenesis has been proposed as new pathways contributing to DM1 pathogenesis.

Between 5 and 37 CUG repeats is considered normal, while individuals with between 38 and 49 repeats are considered to have a pre-mutation and are at risk of having children with further expanded repeats and, therefore, symptomatic disease. Individuals with greater than 50 repeats are almost invariably symptomatic. Patients suffering from DM1 typically have CTG repeat expansions ranging from 50 to more than 2500. Longer repeats are usually associated with earlier onset and more severe disease. The number of repeats increases over successive generations and provides the molecular basis for the anticipation phenomenon observed in DM1 families.

DMPK alleles with greater than 37 repeats are unstable and additional trinucleotide repeats may be inserted during cell division in mitosis and meiosis. Consequently, the children of individuals with premutations or mutations inherit DMPK alleles which are longer than their parents and therefore are more likely to be affected or display an earlier onset and greater severity of the condition, a phenomenon known as anticipation. Interestingly, paternal transmission of the condition is very uncommon, possibly due to selection pressures against sperm with expanded repeats, but anticipation tends to be less severe than in cases of maternal inheritance.

The term “DM1 patient” or “subject having DM1” as used herein refers to a subject having a mutation in the DMPK gene known as a trinucleotide repeat expansion. Typically, DM1 patients have at least 50 CTG repeats in the trinucleotide repeat expansion of the DMPK gene. Subjects having 35 to 49 CTG repeats have not been reported to develop DM1, but their children are at risk of having the disorder if the number of CTG repeats increases. Repeat lengths from 35 to 49 are called pre-mutations. The term “subject having a pre-mutation for DM1” refers to a subject having 35 to 49 CTG repeats in the trinucleotide repeat expansion of the DMPK gene. In an embodiment, the term “DM1 patient” or “subject having DM1” refers to a subject having at least 50 CTG repeats in the trinucleotide repeat expansion of the DMPK gene. In another embodiment, the term “DM1 patient” or “subject having DM1” refers to a subject having at least 35 CTG repeats in the trinucleotide repeat expansion of the DMPK gene. In certain embodiments, the term “DM1 patient” or “subject having DM1” may thus encompass subjects having pre-mutations, which are generally (still) asymptomatic.

The term “myotonic dystrophy protein kinase”, “DMPK”, or “dystrophia myotonica-protein kinase” generally refers to a protein expressed predominantly in skeletal muscle. The gene is located on the long arm of chromosome 19. The cytogenetic location of the DMPK gene is 19q13.3.

The present inventor have here for the first time demonstrated that genetic correction of DM1 with a high efficiency, in particular by designer nucleases is feasible. As DM1 is an autosomal dominant genetic disorder, it may be expected that the present invention may also be applicable to other types of autosomal dominant genetic disorders. Therefore, in certain embodiments, when referring to DM1, such may be replaced by another autosomal dominant genetic disorder. Accordingly, in certain embodiments, when referring to DM1, such may be replaced by a disorder selected from the group comprising or consisting of Acropectoral syndrome, Acute intermittent porphyria, Adermatoglyphia, Albright's hereditary osteodystrophy, Arakawa's syndrome II, Aromatase excess syndrome, Autosomal dominant cerebellar ataxia, Axenfeld syndrome, Bethlem myopathy, Birt-Hogg-Dubé syndrome, Boomerang dysplasia, Branchio-oto-renal syndrome, Buschke-Ollendorff syndrome, Camurati-Engelmann disease, Central core disease, Collagen disease, Collagenopathy, types II and XI, Congenital distal spinal muscular atrophy, Congenital stromal corneal dystrophy, Costello syndrome, Currarino syndrome, Darier's disease, De Vivo disease, Dentatorubral-pallidoluysian atrophy, Dermatopathia pigmentosa reticularis, DiGeorge syndrome, Dysfibrinogenemia, Transthyretin-related hereditary amyloidosis, Familial atrial fibrillation, Familial hypercholesterolemia, Familial male-limited precocious puberty, Feingold syndrome, Felty's syndrome, Flynn-Aird syndrome, Gardner's syndrome, Gillespie syndrome, Gray platelet syndrome, Greig cephalopolysyndactyly syndrome, Hajdu-Cheney syndrome, Hawkinsinuria, Hay-Wells syndrome, Hereditary elliptocytosis, Hereditary hemorrhagic telangiectasia, Hereditary mucoepithelial dysplasia, Hereditary spherocytosis, Holt-Oram syndrome, Huntington's disease, Hypertrophic cardiomyopathy, Hypoalphalipoproteinemia, Jackson-Weiss syndrome, Keratolytic winter erythema, Kniest dysplasia, Kostmann syndrome, Langer-Giedion syndrome, Larsen syndrome, Liddle's syndrome, Marfan syndrome, Marshall syndrome, Medullary cystic kidney disease, Metachondromatosis, Miller-Dieker syndrome, MOMO syndrome, Monilethrix, Multiple endocrine neoplasia, Multiple endocrine neoplasia type 1, Multiple endocrine neoplasia type 2, Multiple endocrine neoplasia type 2b, Myelokathexis, Myotonic dystrophy, Naegeli-Franceschetti-Jadassohn syndrome, Nail-patella syndrome, Noonan syndrome, Oculopharyngeal muscular dystrophy, Pachyonychia congenita, Pallister-Hall syndrome, PAPA syndrome, Papillorenal syndrome, Parastremmatic dwarfism, Pelger-Huet anomaly, Peutz-Jeghers syndrome, Piebaldism, Platyspondylic lethal skeletal dysplasia, Torrance type, Popliteal pterygium syndrome, Porphyria cutanea tarda, RASopathy, Reis-Bucklers corneal dystrophy, Romano-Ward syndrome, Rosselli-Gulienetti syndrome, Roussy-Lévy syndrome, Rubinstein-Taybi syndrome, Saethre-Chotzen syndrome, Schmitt Gillenwater Kelly syndrome, Short QT syndrome, Singleton Merten syndrome, Spinal muscular atrophy with lower extremity predominance, Spinocerebellar ataxia, Spinocerebellar ataxia type-6, Spondyloepimetaphyseal dysplasia, Strudwick type, Spondyloepiphyseal dysplasia congenita, Spondyloperipheral dysplasia, Stickler syndrome, Tietz syndrome, Timothy syndrome, Treacher Collins syndrome, Tuberous sclerosis, Upington disease, Variegate porphyria, Vitelliform macular dystrophy, Von Hippel-Lindau disease, Von Willebrand disease, Wallis-Zieff-Goldblatt syndrome, WHIM syndrome, White sponge nevus, Worth syndrome, Zaspopathy, Zimmermann-Laband syndrome, Zori-Stalker-Williams syndrome. It will be appreciated that the target sequences and constructs as described herein for DM1 can be adapted accordingly to accommodate genetic correction of the above listed disorders.

The term “induced pluripotent stem cells” or “iPS” refers to pluripotent stem cell that can be generated directly from adult cells, in particular somatic cells. iPS may for instance be generated as described in Yamanaka et al. 2006 (Cell 126, 663-676), Yamanaka et al. 2007 (Cell 131, 861-872) and Lin et al. 2009 (Nature Methods 6, 805-808). Similar to embryonic stem cells (ESCs), iPS show unlimited self-renewal and demonstrated pluripotency by contributing to lineages from all three germ layers in the context of embryoid bodies, teratomas, fetal chimeras. By means of further guidance, iPS may be generated from somatic cells by expressing Oct4, Sox2, cMyc, and Klf4. Primary cells may be transduced or transfected by any means known in the art, such as for instance by viral vectors such as retroviral and lentiviral vectors, electroporation with plasmids encoding Myc, Klf4, Oct4 and Sox2, which adequately express these reprogramming factors. The skilled person will appreciate that while this combination of reprogramming factors is most conventional in producing iPS, each of the factors can be functionally replaced by related transcription factors, miRNAs, small molecules, or even non-related genes such as lineage specifiers. Characteristic pluripotency markers for iPS cells are among others OCT4, SOX2, NANOG, hTERT, SSEA4 etc. Verification of the expression of these markers may validate the successful generation of iPS. In a preferred embodiment, the iPS as referred to herein are mammalian iPS, preferably human iPS. Accordingly, cells originating from a subject having myotonic dystrophy type 1 (DM1) in certain embodiments refers to cells originating from a mammalian subject having myotonic dystrophy type 1 (DM1), preferably a human subject having myotonic dystrophy type 1 (DM1).

Except when noted differently, the terms “subject” or “patient” are used interchangeably and refer to animals, preferably vertebrates, more preferably mammals, and specifically includes human patients and non-human mammals. “mammalian” subjects include, but are not limited to, humans, domestic animals, commercial animals, farm animals, zoo animals, sport animals, pet and experimental animals such as dogs, cats, guinea pigs, rabbits, rats, mice, horses, cattle, cows; primates such as apes, monkeys, orang-utans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Accordingly, “subject” or “patient” as used herein means any mammalian patient or subject to which the compositions of the invention can be administered. Preferred patients or subjects are human subjects.

The present methods and protocols may preferably depart from iPS which are “undifferentiated”, i.e., wherein a substantial proportion (for example, at least about 60%, preferably at least about 70%, even more preferably at least about 80%, still more preferably at least about 90% and up to 100%) of cells in the stem cell population display characteristics (e.g., morphological features and/or markers) of undifferentiated iPS cells, clearly distinguishing them from cells undergoing differentiation. Undifferentiated iPS cells are generally easily recognised by those skilled in the art, and may appear in the two dimensions of a microscopic view with high nuclear/cytoplasmic ratios and prominent nucleoli, may grow as compact colonies with sharp borders. It is understood that colonies of undifferentiated cells within the population may often be surrounded by neighbouring cells that are more differentiated. Nevertheless, the undifferentiated colonies persist when the population is cultured or passaged under appropriate conditions known per se, and individual undifferentiated cells constitute a substantial proportion of the cell population. By means of further guidance, iPS identity may also be verified by various cellular biological properties. iPS may for instance express typical stem cell markers, such as SSEA-3, SSEA-4, TRA-1-60, TRA-1-81, TRA-2-49/6E, and Nanog. iPS typically also demonstrate high telomerase activity and express hTERT. Further, iPS are mitotically active, actively self-renewing, proliferating, and dividing at a rate equal or similar to ESCs.

In an aspect, the invention relates to cells originating from a subject having myotonic dystrophy type 1 (DM1), in which cells the expression of an expanded repeat RNA (CUGexp) of the dystrophy myotonic-protein kinase (DMPK) gene is reduced or eliminated. As used herein, the reduction or elimination of the expanded repeat RNA of the DMPK gene in cells may also be referred to as genetically corrected cells.

According to the invention, any cell type derived from a subject having DM1 may be used in the methods for genetic targeting and compositions as described herein. The genetic targeting may be performed on any such cell type derived from a subject having DM1, but also on cells derived from these, such as iPS. Accordingly, in certain embodiments, the genetic targeting as described herein may be performed on iPS cells derived from any cell type originating from a subject having DM1. In certain embodiments, the genetic targeting as described herein may also be performed on further downstream cells derived from the cells originating from a subject having DM1, such as cells derived from iPS cells which in their turn are derived from cells originating from a subject having DM1. Such downstream cells may be precursor or progenitor cells, which are to a certain extent already lineage committed, but nevertheless retain a certain degree of proliferative capacity, as is known in the art. In certain embodiments, the cells to be used for the genetic targeting as described herein may be myogenic cells, which may in certain embodiments be primary myogenic cells or iPS-derived myogenic cells. In certain other embodiments, the cells to be used for the genetic targeting as described herein may be neurogenic cells, which may in certain embodiments be primary neurogenic cells or iPS-derived neurogenic cells. In certain embodiments, the cells as referred to herein are muscle cells (e.g. skeletal muscle cells), heart cells (e.g. cardiomyocytes), eye cells (e.g. lens epithelial cells) or brain cells (e.g. neurons), progenitor or precursor cells from which these cells are the progeny, iPS cells derived therefrom (i.e. from the precursor/progenitor cells, for instance myoblasts or mesoangioblasts, or the progeny of these cells), or the progeny thereof, such as iPS-derived progenitor or precursor cells, which may in certain embodiments be myogenic or neurogenic progenitors/precursors, or their partially or fully differentiated progeny.

According to certain embodiments, the invention relates to the use of the above described cells (i.e. the cells which have been genetically corrected) for the treatment of DM1. In certain embodiments, the invention relates to the above described cells (i.e. the cells which have been genetically corrected) for use in the treatment of DM1. In certain embodiments, the invention relates to a method for treating DM1, comprising administering the above described cells (i.e. the cells which have been genetically corrected). In certain embodiments, the invention relates to the use of the above described cells (i.e. the cells which have been genetically corrected) for the manufacture of a medicament for the treatment of DM1.

As used herein, “myoblasts” and “mesoangioblasts” refer to primary cells of mesodermal origin. Both myoblasts and mesoangioblasts are progenitor cells. These cells are multipotent and can differentiate or can be induced to differentiate into a variety of cell types, such as for instance myogenic or cardiomyogenic differentiation into for instance myocytes. As used herein “myoblast-like” and mesoangioblast-like” cells refer to cells having morphological or functional characteristics similar to respectively myoblasts or mesoangioblasts. Importantly, myoblast-like and mesoangioblast-like cells are also capable of myogenic or cardiomyogenic differentiation into for instance myocytes. Similarly, “muscle-like” or “neuronal-like” cells refer to cells having morphological or functional characteristics similar to respectively muscle cells or neuronal cells.

Characteristic markers for the specific cell types as used herein can verify the identity of the cells. HIDEMs or Mesangioblastscan for instance be characterized as follows: CD13 positive, CD31 negative, CD44 positive, CD56 negative, CD49b positive, CD45 negative, SSEA4 negative. Accordingly, in certain embodiments, the cells as used herein may be characterized by one or more of the following: CD13 positive, CD31 negative, CD44 positive, CD56 negative, CD49b positive, CD45 negative, SSEA4 negative, preferably at least two of the above, more preferably at least 3, even more preferably at least 4, 5, or 6, most preferably all of the above. Mature Myotubes or myocytes can for instance be characterized by expression of Myosin Heavy Chain (MyHC). Myoblasts (or myoblast-like cells) can for instance be characterized by expression of MyoD and/or myogenin, preferably both. Alternatively, myoblasts can for instance be identified by expression of one or more of the following markers, Acetylcholinesterase (AChE), ADAM12, alpha- and beta-tropomyosin (pT), normally concentrated in myotubes. PMID: 6301863, beta-Enolase, CD56, Desmin, Lactate Dehydrogenase (LDH), M-Cadherin (muscle cadherin), M-Calpain, M-CAM (melanoma cell adhesion molecule), MRF4 (myogenic/muscle regulating factor-4), Myf-5 (muscle regulatory factor-5), MyoD, Myogenin, Myosin, N-Cadherin (neural cadherin), Phosphoprotein (pp(65; 4.5)), Pax3, Pax7, PK-K (K-isozyme of pyruvate kinase), PK-M (M-isozyme of pyruvate kinase), Tbx3, Titin, one or more of which may be used in certain embodiments to identify myoblasts or myoblast-like cells as described herein. Preferably at least two of the above, more preferably at least 3, even more preferably at least 4, 5, or 6, such as all of the above markers are present on the myoblasts or myoblast-like cells as described herein.

The present methods and protocols may depart from such myoblast-like or mesoangioblast-like cells or alternatively from neuronal or neurogenic cells which are not pluripotent anymore, but which are neither terminally differentiated, i.e., wherein a substantial proportion (for example, at least about 60%, preferably at least about 70%, even more preferably at least about 80%, still more preferably at least about 90% and up to 100%) of cells cell population display characteristics (e.g., morphological features and/or markers) clearly distinguishing them from the iPS and/or cells undergoing terminal differentiation.

The terms “progenitor” or “precursor” refer generally to an unspecialized or relatively less specialized and proliferation-competent cell which can under appropriate conditions give rise to at least one relatively more specialized cell type, such as inter alia to relatively more specialized progenitor cells or eventually to terminally differentiated cells, i.e., fully specialized cells that may be post-mitotic. A progenitor or stem cell is said to “give rise” to another, relatively more specialized cell when, for example, the progenitor or stem cell differentiates to become said other cell without previously undergoing cell division, or if said other cell is produced after one or more rounds of cell division and/or differentiation of the progenitor or stem cell.

As used herein, the terms “differentiation”, “differentiating” or derivatives thereof denote the process by which an unspecialized or relatively less specialized cell, such as, for example, stem cell or progeny thereof, becomes relatively more specialized. In the context of cell ontogeny, the adjective “differentiated” is a relative term. Hence, a “differentiated cell” is a cell that has progressed further down a certain developmental pathway than the cell it is being compared with. The differentiated cell may, for example, be a terminally differentiated cell, i.e., a fully specialized cell capable of taking up specialized functions in various tissues or organs of an organism, which may but need not be post-mitotic; or the differentiated cell may itself be a progenitor cell within a particular differentiation lineage which can further proliferate and/or differentiate. A relatively more specialized cell may differ from an unspecialized or relatively less specialized cell in one or more demonstrable phenotypic characteristics, such as, for example, the presence, absence or level of expression of particular cellular components or products, e.g., RNA, proteins or other substances, activity of certain biochemical pathways, morphological appearance, proliferation capacity and/or kinetics, differentiation potential and/or response to differentiation signals, electrophysiological behavior, etc., wherein such characteristics signify the progression of the relatively more specialized cell further along the said developmental pathway. The term “(cardio)myogenic differentiation”, “differentiation into myoblast-like or mesoangioblast-like cells” or the likes means the formation of (cardio)myocytes from stem cells, such as iPS. Formation of (cardio)myocytes is defined by the formation of contracting embryoid bodies (EBs), contracting seeded cells, immune cytological staining for cardiomyocyte specific marker, and expression of (cardio)myocyte specific marker. Such differentiation may be accomplished by subjecting cells to a “medium permissive to differentiation of stem cells”, which means that the medium does not contain components, in sufficient quantity, which would suppress stem cell differentiation or would cause maintenance and/or proliferation of stem cells in undifferentiated or substantially undifferentiated state. By means of illustration, such components absent from the medium may include leukaemia inhibitory factor (LIF), basic fibroblast growth factor (b-FGF), and/or embryonic fibroblast feeders or conditioned medium of such feeders. The above applies equally mutatis mutandis to “neurogenic differentiation” or “neuronal differentiation”.

In a further aspect, the invention relates to precursor cells derived from, differentiated from, obtained from, or generated from the iPS as defined herein, i.e. precursor cells derived from iPS originating from a subject having DM1. In related aspects, the invention relates to a method for differentiating the iPS as defined herein into precursor cells; or the use of the iPS as defined herein for differentiating, generating, obtaining, or giving rise to precursor cells. In certain embodiments, the precursor cells are myogenic precursor cells. In certain other embodiments, the precursor cells are neurogenic precursor cells. As used herein, “myogenic precursor cells” is synonymous with “myogenic progenitor cells”, and refers to cells which are capable of directly differentiating into muscle cells, such as myocytes, or indirectly into other cell types which in their turn may directly or indirectly differentiate into muscle cells. In certain embodiments, the iPS as defined herein may undergo myogenic differentiation such as to form myoblast-like cells or mesoangioblast-like cells. Accordingly, in certain embodiments, the invention relates to myoblast-like cells or mesoangioblast-like cells derived from, differentiated from, obtained from, or generated from the iPS as defined herein, i.e. myogenic precursor cells derived from iPS originating from a subject having DM1. In related embodiments, the invention relates to a method for differentiating the iPS as defined herein into myoblast-like cells or mesoangioblast-like cells; or the use of the iPS as defined herein for differentiating, generating, obtaining, or giving rise to myoblast-like cells or mesoangioblast-like cells. As used herein, “neurogenic precursor cells” is synonymous with “neurogenic progenitor cells”, and refers to cells which are capable of directly differentiating into neuronal cells, such as neurons, or indirectly into other cell types which in their turn may directly or indirectly differentiate into neuronal cells. In certain embodiments, the iPS as defined herein may undergo neurogenic differentiation such as to form neuron-like cells. Accordingly, in certain embodiments, the invention relates to neuron-like cells or neuronal-like cells derived from, differentiated from, obtained from, or generated from the iPS as defined herein, i.e. neuronal or neurogenic precursor cells derived from iPS originating from a subject having DM1. In related embodiments, the invention relates to a method for differentiating the iPS as defined herein into neuronal- or neurogenic-like cells or neuron-like cells; or the use of the iPS as defined herein for differentiating, generating, obtaining, or giving rise to neuron- or neuronal-like cells or neurogenic-like cells.

In another aspect, the present invention relates to cells originating from a subject having myotonic dystrophy type 1 (DM1), for example, but not limited to the iPS as defined herein, or the precursor cells derived therefrom (such as myogenic or neurogenic precursors, as described herein elsewhere, in which the expression of an expanded repeat RNA (CUGexp) of the dystrophy myotonic-protein kinase (DMPK) gene is reduced or eliminated. As defined herein elsewhere, DM1 patients have an expanded repeat RNA in the 3′UTR of the DMPK gene, in which the CTG triplet is repeated in a greater number than in subjects not having DM1. Upon transcription of such DM1 DMPK allele, a transcript is generated which typically comprises at least 35 CUG repeats, preferably at least 50 CUG repeats. According to certain embodiments, the expression of such mutant DMPK allele having at least 35 CTG repeats, preferably at least 50 CTG repeats is reduced or eliminated. As referred to herein, “reduced” or “eliminated” expression encompasses both a reduced or eliminated transcription of the mutant DMPK gene, such that less or (substantially) no mRNA is formed, as well as an increased breakdown of the mutant mRNA, such as to eliminate partially or (substantially) completely already formed mRNA or increase mRNA turnover. Alternatively, for instance partial or (substantially) complete sequestration of the mutant mRNA is also envisaged, in order for the mRNA to be prevented from exploiting its pathogenicity, such as to prevent interference of the mutant mRNA with splicing. A reduction of the expression of the mutant mRNA preferably refers to at least 10% (on weight basis) less functional pathogenic mRNA being present in the cell compared to non-reduced conditions, more preferably at least 30%, even more preferably at least 50%, yet more preferably at least 70%. Elimination of the expression of the mutant mRNA preferably refers to at least 80% (on weight basis) less functional pathogenic mRNA being present in the cell compared to non-reduced conditions, more preferably at least 90%, even more preferably at least 95%, such as 98%, or 100% or substantially 100%. Means for reducing or eliminating expression of mRNA are well known in the art, all of which can be used in certain embodiment of the invention.

In a preferred embodiment, the reduction or elimination of the expression of said expanded repeat RNA (CUGexp) of the DMPK gene is effected by introducing in said cells a designer nuclease specifically targeting the DMPK gene or locus, preferably a designer endonuclease, more preferably a designer endodeoxyribonuclease. Preferably, the nuclease generates double strand breaks in DNA, such as genomic DNA.

As used herein, a “designer nuclease” is a multicomponent polypeptide, typically comprising a site specific polynucleotide binding moiety, which may be a polynucleotide recognizing peptide or alternatively an oligo- or polynucleotide, and which is attached to or associated with a nuclease moiety. The polynucleotide binding moiety targets the nuclease to a specific site on the polynucleotide, such that a site-specific cut can be made in the polynucleotide. The nuclease itself, were it not for being fused to or associated with the site specific polynucleotide binding moiety, does not possess site-specificity. Designer nucleases are generally engineered in order to provide target specific recognition and cleavage. Accordingly, “specifically targeting the DMPK gene” refers to site-specific binding of the nuclease (i.e. the site specific polynucleotide binding moiety which is fused to or associated with the nuclease) to a sequence of the DMPK gene. Such sequence may be a regulatory sequence, such as the DMPK promoter, an intron, an exon, an intron/exon boundary, 5′-UTR, 3′-UTR, etc. By inserting a DNA double-strand break (DSB) into the target locus rare-cutting designer nucleases activate DNA repair, which can be harnessed to knockout genes or to promote gene targeting. At least five families of designer nucleases have been documented so far: Zinc Finger Nucleases (ZFNs), meganucleases (MNs), chemical nucleases, Transcription Activator-Like Nucleases (TALENs), and CRISPR/Cas nucleases. All these nucleases may be used according to certain embodiments of the invention.

In the methods of the present invention be it at the promoter region of the DMPK gene or the 3′UTR where the CTG repeat regions are removed, the healthy or the mutant allele can both be targeted. Because the homologous sequence in the guideRNA are the same in both health or mutant allele. The only difference is the distance between two guideRNA flanking the CTG repeat in a healthy allele is smaller than the mutant allele since the mutant allele has the long stretch of CTG repeats between the two targeting sites of the guideRNA.

The present invention, relating to the reduction of CTG repeats in the DMPK gene in the genome of a cell of a DM1 patient is illustrated with the TALEN and CRISPR/Cas technology. Based on the teaching of the present application, the skilled person can apply this teaching to other genome specific nuclease systems such as Zinc Finger Nucleases (ZFNs), meganucleases (MNs), chemical nucleases.

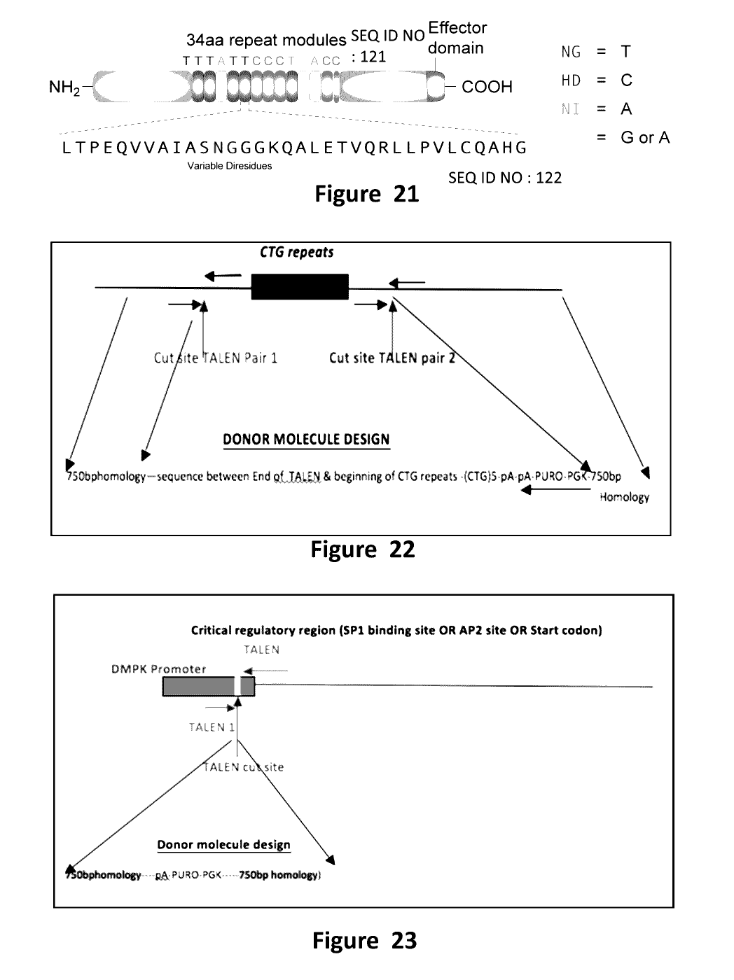

In a preferred embodiment, the nuclease comprises a designer transcription activator-like effector nuclease (dTALEN or TALEN). TALENs are composed of the FokI nuclease fused to the TALE domains that determine the specificity of TALEN binding. The TALEs central domain contains a variable number of tandem, 34-amino acid repeats. This repeat domain was previously shown to bind specific DNA sequences in promoter regions of target genes. Amino acid sequences of the repeats are conserved, except for two adjacent highly variable residues (at positions 12 and 13) that are specificity determinants defined as the repeat-variable diresidue (RVD). A simple one-to-one code had been deduced relating specific diamino acids in the repeat unit to specific nucleotides in the DNA target. Remarkably, the RVDs of TALE correspond directly to the nucleotides in their target sites, one RVD to one nucleotide. The generic TALE structure and TALE code is shown in FIG. 21. As the FokI nuclease needs to dimerize in order to generate double strand breaks, two TALEs—each fused to a FokI—are needed: a left TALE and a right TALE. Accordingly, a functional dTALEN as used herein, may comprise and preferably comprises a left TALE and a right TALE (each fused to FokI), each of which is capable of recognizing respectively a left target (or left TALE target) sequence and a right target (or right TALE target) sequence.

According to certain embodiments, the dTALEN (interchangeably used with TALEN or TALE, and which may comprise the left TALE and the right TALE) as referred to herein is capable of targeting or binding to the DMPK locus. The term “locus” is known in the art. By means of further guidance, the DMPK locus refers to the physical location of the DMPK gene on the chromosome. The DMPK locus encompasses both the transcribed parts of the gene, being it introns, exons, 5′-UTR, or 3′ UTR, as well as the associated regulatory sequences, such as promoters, enhancers, etc.

In a preferred embodiment, the nuclease comprises an RNA based designer nuclease, preferably a clustered regulatory interspaced short palindromic repeat (CRISPR)/Cas-based RNA-guided DNA endonuclease. The CRISPR/Cas system finds its origin in prokaryotes, which have evolved an adaptive defense mechanism that uses CRISPR, together with Cas proteins, to renders them resistance to invading viruses and plasmids. The original prokaryotic type II CRISPR-Cas system results in the specific cleavage of incoming exogenous DNA fragments by the Cas9 endonuclease that is directed to this DNA sequence using complementary RNAs. Based on this CRISPR-Cas system, RNA-Guided Endonucleases (RGENs) can be designed that can be used to perform targeted genome editing at the desired loci. Recent work has shown that CRISPR/Cas systems can be engineered to direct targeted double-stranded DNA breaks in vitro to specific sequences by using a single “guide RNA” with complementarity to the DNA target site and a Cas9 nuclease (Jinek et al., Science (2012) Science 337, 816-821). This targetable Cas9-based system also works efficiently in cultured human cells (Mali et al. (2013) Science 339, 823-826; Cong et al., (2013) Science 339, 819-823). In essence, the CRISPR/Cas system uses a single or double synthetic non-coding nucleotide guide RNA (gRNA) that uses 20 variable nucleotides at its 5′ end to base pair with a target polynucleotide region. The remaining gRNA scaffold interacts with and redirects the Cas9 nuclease to the target site. Target sequences are generally at least 20 bp in length and must be followed by an appropriate protospace-adjacent motif (PAM) on their 3′ end. These designer nucleases then typically activate DNA repair resulting in a specific gene knockout through non-homologous end-joining (NHEJ). Moreover, in the presence of a homologous gene sequence, targeted integration of the gene of interest can be achieved in the target locus by homologous recombination.

As used herein, the terms “CRISPR” and “guide RNA” or “gRNA” are to a certain extent used interchangeably. Whereas “guide RNA” or “gRNA” specifically relate to an RNA sequence, “CRISPR” may relate both to an RNA or DNA sequence. However, when referring to “CRISPR” or “guide RNA/gRNA” in a specific context, these terms relate to a similar sequence, being it either DNA or RNA, in which T is replaced by U. As used herein, and unless specified otherwise, when referring to a CRISPR sequence, such sequence may be single stranded or double stranded, and may be DNA or RNA.

FIG. 33 shows a representation of the complex formed by guide RNA molecule, Cas9 nuclease and genomic DNA. The guide RNA molecule consists of a “target sequence” specifically binding to genomic target DNA and a “scaffold sequence” which forms hairpins and binds with the Cas9 nuclease protein.

The length of the target sequence of the guide RNA which specifically binds with the genomic ranges from 17 to 23 nucleotides.

As used herein, “Cas” refers to CRISPR associated nuclease. Cas may be obtained from a variety of prokaryotic sources. In certain preferred embodiments, the Cas as referred to herein is Cas9, preferably a codon-optimized Cas or Cas9, such as a human codon-optimized Cas or Cas9. In certain embodiments, the Cas as referred to herein has a sequence as set forth in SEQ ID NO: 44, or a homologue, functional variant or functional fragment thereof. In certain embodiments, the Cas as referred to herein is cloned into an expression vector having a sequence as set forth in SEQ ID NO: 59, or a homologue, functional variant or functional fragment thereof. In addition, one amino acid mutation at position D 10A in Cas9 results in the inactivation of the nuclease catalytic activity and converts Cas9 to a “nickase” enzyme that makes single-stranded breaks at the target site, which may also be used, although not preferred, according to certain embodiments of the invention.

According to certain embodiments, the CRISPR as referred to herein is capable of targeting or binding to the DMPK locus. The term “locus” is known in the art. By means of further guidance, the DMPK locus refers to the physical location of the DMPK gene on the chromosome. The DMPK locus encompasses both the transcribed parts of the gene, being it introns, exons, 5′-UTR, or 3′ UTR, as well as the associated regulatory sequences, such as promoters, enhancers, etc.

A “regulatory sequence” or “regulatory element” as used herein refers to transcriptional control elements, in particular non-coding cis-acting transcriptional control elements, capable of regulating and/or controlling transcription of a gene, in particular tissue-specific transcription of a gene. Regulatory elements comprise at least one transcription factor binding site (TFBS), more in particular at least one binding site for a tissue-specific transcription factor, most particularly at least one binding site for a liver-specific transcription factor. Typically, regulatory elements as used herein increase or enhance promoter-driven gene expression when compared to the transcription of the gene from the promoter alone, without the regulatory elements. Thus, regulatory elements particularly comprise enhancer sequences, although it is to be understood that the regulatory elements enhancing transcription are not limited to typical far upstream enhancer sequences, but may occur at any distance of the gene they regulate. Indeed, it is known in the art that sequences regulating transcription may be situated either upstream (e.g. in the promoter region) or downstream (e.g. in the 3′UTR) of the gene they regulate in vivo, and may be located in the immediate vicinity of the gene or further away. Of note, although regulatory elements as disclosed herein typically are naturally occurring sequences, combinations of (parts of) such regulatory elements or several copies of a regulatory element, i.e. non-naturally occurring sequences, are themselves also envisaged as regulatory element. Regulatory elements as used herein may be part of a larger sequence involved in transcriptional control, e.g. part of a promoter sequence. However, regulatory elements alone are typically not sufficient to initiate transcription, but require a promoter to this end. As used in the application, the term “promoter” refers to nucleic acid sequences that regulate, either directly or indirectly, the transcription of corresponding nucleic acid coding sequences to which they are operably linked (e.g. a transgene or endogenous gene). A promoter may function alone to regulate transcription or may act in concert with one or more other regulatory sequences (e.g. enhancers or silencers). In the context of the present application, a promoter is typically operably linked to regulatory elements to regulate transcription of a transgene.

In certain embodiments, the dTALEN as referred to herein targets/binds or is capable of targeting/binding to the DMPK promoter. In other embodiments, the dTALEN as referred to herein targets/binds or is capable of targeting/binding to a DMPK enhancer. In other embodiments, the dTALEN as referred to herein targets/binds or is capable of targeting/binding to a DMPK exon. In other embodiments, the dTALEN as referred to herein targets/binds or is capable of targeting/binding to a DMPK intron. In other embodiments, the dTALEN as referred to herein targets/binds or is capable of targeting/binding to a DMPK exon/intron boundary. In other embodiments, the dTALEN as referred to herein targets/binds or is capable of targeting/binding to a DMPK exon encompassing the 5′UTR. In other embodiments, the dTALEN as referred to herein targets/binds or is capable of targeting/binding to a DMPK exon encompassing the 3′UTR. In other embodiments, the dTALEN as referred to herein targets/binds or is capable of targeting/binding to the DMPK CTG repeat. In other embodiments, the dTALEN as referred to herein targets/binds or is capable of targeting/binding to the junction between the DMPK gene sequence and the CTG trinucleotide repeat, preferably the expanded CTG trinucleotide repeat. As used herein, the term “expanded CTG trinucleotide repeat” refers to the CTG trinucleotide repeat having at least 35 CTG trinucleotides, preferably at least 50 CTG trinucleotides. In other embodiments, the dTALEN as referred to herein targets/binds or is capable of targeting/binding to a transcription factor binding site in the DMPK promoter. In other embodiments, the dTALEN as referred to herein targets/binds or is capable of targeting/binding to the AP-2 binding site in the DMPK promoter. In other embodiments, the dTALEN as referred to herein targets/binds or is capable of targeting/binding to the SP1 binding site in the DMPK promoter. In other embodiments, the dTALEN as referred to herein targets/binds or is capable of targeting/binding to the start codon of the DMPK gene. In other embodiments, the dTALEN as referred to herein is encoded by a sequence comprising, consisting of, or consisting essentially of a polynucleic acid sequence as set forth in any of SEQ ID NOs: 1 or 3, preferably both (wherein SEQ ID NO: 1 corresponds to the left TALE and SEQ ID NO: 3 corresponds to the right TALE), the complement thereof, or the reverse complement thereof, wherein T may be replaced by U. The present invention also relates to a polynucleic acid sequence comprising a dTALEN sequence as defined above, the complement thereof, or the reverse complement thereof, wherein T may be replaced by U. The invention further relates to a polynucleic acid sequence comprising, consisting of, or consisting essentially of a sequence as set forth in any of SEQ ID NOs: 1, 3, 5, 6, 7, 8, 9, 10-19, 26, or 27, the complement thereof, or the reverse complement thereof, wherein T may be replaced by U. In another embodiment, the invention relates to a dTALEN as referred to herein comprising, consisting of, or consisting essentially of a polypeptide sequence as set forth in any of SEQ ID NOs: 2 or 4, preferably both (wherein SEQ ID NO: 2 corresponds to the left TALE and SEQ ID NO: 4 corresponds to the right TALE). The present invention also relates to a polypeptide sequence comprising a dTALEN sequence as defined above. The invention further relates to a polypeptide sequence comprising, consisting of, or consisting essentially of a sequence as set forth in any of SEQ ID NOs: 2 or 4. The invention further relates to a dTALEN as referred to herein, comprising, consisting of, or consisting essentially of a polypeptide sequence encoded by a polynucleic acid sequence as set forth in any of SEQ ID NOs: 1 or 3. The skilled person will understand that additional dTALENs (such as combinations of left and right TALEs, or combinations of TALE pairs each composed of a left and right TALE) may be designed which recognize specific additional target sequences, based on the consensus TALE structure and target recognition code (see also FIG. 21).

Particularly preferred combinations of left and right TALE target sequences (from which the skilled person can easily design the left and right TALE which recognized these sequences) are listed in Table 2.

TABLE 2

| Left TALEN target sequence | Right TALEN |
|---|---|
| (SEQ ID NO) | target sequence |
| 5 | 6 |
| 10 | 11 |
| 12 | 13 |
| 14 | 15 |
| 16 | 17 |
| 18 | 19 |

Accordingly, in certain embodiments, the invention relates to dTALEN comprising a left and right TALEN capable of recognizing a target sequence respectively as indicated in Table 2.

Particularly suited dTALEN pairs (each comprising a left and right TALEN) according to the present invention are capable of recognizing the TALE target sequence as listed in Table 3.

TABLE 3

| 5′TALEN target sequence | |
|---|---|
| (left and right | 3′ TALEN target sequence |
| SEQ ID NO, respectively) | (left and right SEQ ID NO, respectively) |
| 12, 13 | 10, 11 |

Accordingly, in certain embodiments, the invention relates to a dTALEN pair each dTALEN comprising a left and right TALEN capable of recognizing a target sequence respectively as indicated in Table 3.

In another aspect, the invention relates to a DMPK dTALEN target sequence as set forth in any of SEQ ID NOs: 5, 6, or 10-19. Accordingly, in certain embodiments, the invention relates to a polynucleic acid sequence as set forth in any of SEQ ID NOs: 5, 6, or 10-19.

In another aspect, the invention relates to a dTALEN as defined herein which is capable of binding to a target sequence as defined above. Accordingly, in certain embodiments, the invention relates to a dTALEN, as defined herein, which is capable of binding to a polynucleic acid sequence as set forth in any of SEQ ID NOs: 5, 6, or 10-19. In certain embodiments, the invention relates to a polynucleic acid sequence of a dTALEN, as defined herein, capable of binding to a polynucleic acid sequence as set forth in any of SEQ ID NOs: 5, 6, or 10-19. In other embodiments, the invention relates to a polynucleic acid sequence encoding a polypeptide sequence of a dTALEN, as defined herein, capable of binding to a polynucleic acid sequence as set forth in any of SEQ ID NOs: 5, 6, or 10-19. In other embodiments, the invention relates to a polypeptide sequence of a dTALEN, as defined herein, capable of binding to a polynucleic acid sequence as set forth in any of SEQ ID NOs: 5, 6, or 10-19.

In a further aspect, the invention relates to a vector comprising a polynucleic acid sequence as defined above, such as preferably a vector as set forth in any of SEQ ID NOs: 26 or 27 or 40. In another aspect, the invention relates to a polynucleic acid sequence comprising a sequence as set forth in any of SEQ ID NOs: 1, 3, 5, 6, 7, 8, 9, 10-19, 26, 27, or 40, the complement thereof, or the reverse complement thereof, wherein T may be replaced by U. In a preferred embodiment, the vector as described herein is capable of effecting expression of the polynucleic acid as defined herein, such as the dTALEN as defined herein, or the sequence as set forth in any of SEQ ID NOs: 1, 3, 5, 6, 7, 8, 9, or 10-19. To this end, the polynucleic acid sequences as defined herein generally are operably linked to regulatory sequences which permit transcription of said sequence. In other embodiments, the dTALEN as referred to herein comprises, consist of, or consist essentially of a sequence encoded by a sequence as set forth in any of SEQ ID NOs: 1 or 3, preferably both (wherein SEQ ID NO: 1 corresponds to the left TALE and SEQ ID NO: 3 corresponds to the right TALE), or a sequence having at least 80%, preferably at least 85%, more preferably at least 90%, even more preferably at least 95% sequence identity with a sequence as set forth in any of SEQ ID NOs: 1 or 3, the complement thereof, or the reverse complement thereof, wherein T may be replaced by U. The present invention also relates to a polynucleic acid sequence comprising a dTALEN sequence as defined above, the complement thereof, or the reverse complement thereof, wherein T may be replaced by U. The invention further relates to a polynucleic acid sequence comprising, consisting of, or consisting essentially of a polynucleic acid sequence as set forth in any of SEQ ID NOs: 1, 3, 5, 6, 7, 8, 9, 10-19, 26, 27, or 40, or a sequence having at least 80%, preferably at least 85%, more preferably at least 90%, even more preferably at least 95% sequence identity with a sequence as set forth in any of SEQ ID NOs: 1, 3, 5, 6, 7, 8, 9, 10-19, 26, 27, or 40, the complement thereof, or the reverse complement thereof, wherein T may be replaced by U. The invention further relates to a polypeptide sequence comprising, consisting of, or consisting essentially of a polypeptide sequence as set forth in any of SEQ ID NOs: 2 or 4, or a sequence having at least 80%, preferably at least 85%, more preferably at least 90%, even more preferably at least 95% sequence identity with a sequence as set forth in any of SEQ ID NOs: 2 or 4. In a further aspect, the invention relates to a vector comprising a polynucleic acid sequence as defined above. In another aspect, the invention relates to a polynucleic acid sequence comprising a sequence as set forth in any of SEQ ID NOs: 1, 3, 5, 6, 7, 8, 9, or 10-19, 26, 27, or 40, or a sequence having at least 80%, preferably at least 85%, more preferably at least 90%, even more preferably at least 95% sequence identity with a sequence as set forth in any of SEQ ID NOs: 1, 3, 5, 6, 7, 8, 9, or 10-19, 26, 27, or 40, the complement thereof, or the reverse complement thereof, wherein T may be replaced by U. In a preferred embodiment, the vector as described herein is capable of effecting expression of the polynucleic acid as defined herein, such as the dTALEN as defined herein, or the sequence as set forth in any of SEQ ID NOs: 1, 3, 5, 6, 7, 8, 9, or 10-19, 26, 27, or 40, or a sequence having at least 80%, preferably at least 85%, more preferably at least 90%, even more preferably at least 95% sequence identity with a sequence as set forth in any of SEQ ID NOs: 1, 3, 5, 6, 7, 8, 9, or 10-19, 26, 27, or 40. To this end, the polynucleic acid sequences as defined herein generally are operably linked to regulatory sequences which permit transcription of said sequence. It is to be understood that the variants of the polynucleic acid sequences or the polypeptide sequences of the dTALEN as defined above are still capable of binding to a target sequence as defined herein.

Methods for comparing sequences and determining sequence identity are well known in the art. By means of example, percentage of sequence identity refers to a percentage of identical nucleic acids or amino acids between two sequences after alignment of these sequences. Alignments and percentages of identity can be performed and calculated with various different programs and algorithms known in the art. Preferred alignment algorithms include BLAST (Altschul, 1990; available for instance at the NCBI website) and Clustal (reviewed in Chenna, 2003; available for instance at the EBI website). Preferably, BLAST is used to calculate the percentage of identity between two sequences, such as the “Blast 2 sequences” algorithm described by Tatusova and Madden (1999) FEMS Microbiol Lett 174, 247-250, for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3). The skilled person will understand that sequence identity between two sequences is determined based on the aligned portions of the sequences.

In certain embodiments, the CRISPR as referred to herein targets/binds or is capable of targeting/binding to the DMPK promoter. In other embodiments, the CRISPR as referred to herein targets/binds or is capable of targeting/binding to a DMPK enhancer. In other embodiments, the CRISPR as referred to herein targets/binds or is capable of targeting/binding to a DMPK exon. In other embodiments, the CRISPR as referred to herein targets/binds or is capable of targeting/binding to a DMPK intron. In other embodiments, the CRISPR as referred to herein targets/binds or is capable of targeting/binding to a DMPK exon/intron boundary. In other embodiments, the CRISPR as referred to herein targets/binds or is capable of targeting/binding to a DMPK exon encompassing the 5′UTR. In other embodiments, the CRISPR as referred to herein targets/binds or is capable of targeting/binding to a DMPK exon encompassing the 3′UTR. In other embodiments, the CRISPR as referred to herein targets/binds or is capable of targeting/binding to the DMPK CTG repeat. In other embodiments, the CRISPR as referred to herein targets/binds or is capable of targeting/binding to the junction between the DMPK gene sequence and the CTG trinucleotide repeat, preferably the expanded CTG trinucleotide repeat. As used herein, the term “expanded CTG trinucleotide repeat” refers to the CTG trinucleotide repeat having at least 35 CTG trinucleotides, preferably at least 50 CTG trinucleotides. In other embodiments, the CRISPR as referred to herein targets/binds or is capable of targeting/binding to a transcription factor binding site in the DMPK promoter. In other embodiments, the CRISPR as referred to herein targets/binds or is capable of targeting/binding to the AP-2 binding site in the DMPK promoter. In other embodiments, the CRISPR as referred to herein targets/binds or is capable of targeting/binding to the SP1 binding site in the DMPK promoter. In other embodiments, the CRISPR as referred to herein targets/binds or is capable of targeting/binding to the start codon of the DMPK gene. In other embodiments, the CRISPR as referred to herein comprises, consist of, or consist essentially of a sequence as set forth in any of SEQ ID NOs: 45, 46, 48, 49, 50, 51, 61, 62, 75, 76, 83, or 84, the complement thereof, or the reverse complement thereof, wherein T may be replaced by U. In other embodiments, the CRISPR as referred to herein comprises a target sequence of a guide RNA, consisting of, or consist essentially of a sequence as set forth in any of SEQ ID NOs: 104 to 118, the complement thereof, or the reverse complement thereof, wherein T may be replaced by U. The present invention also relates to a polynucleic acid sequence comprising a CRISPR sequence as defined above, the complement thereof, or the reverse complement thereof, wherein T may be replaced by U. The invention further relates to a polynucleic acid sequence comprising, consisting of, or consisting essentially of a sequence as set forth in any of SEQ ID NOs: 43, 45, 46, 48, 49, 50, 51, 52, 53, 54, 55, 56, 57, 58-62, 75, 76, 83, or 84, the complement thereof, or the reverse complement thereof, wherein T may be replaced by U.

The invention further relates to a polynucleic acid sequence comprising, consisting of, or consisting essentially of a sequence as set forth in any of SEQ ID NOs: 104 to 118, the complement thereof, or the reverse complement thereof, wherein T may be replaced by U.

In a further aspect, the invention relates to a vector comprising a polynucleic acid sequence as defined above. In another aspect, the invention relates to a polynucleic acid sequence comprising a sequence as set forth in any of SEQ ID NOs: 43, 45, 46, 48, 49, 50, 51, 52, 53, 54, 55, 56, 57, 58-62, 75, 76, 83, or 84, the complement thereof, or the reverse complement thereof, wherein T may be replaced by U In another aspect, the invention relates to a polynucleic acid sequence comprising a sequence as set forth in any of SEQ ID NOs: 104, 105, 106, 107, 108, 109, 110, 111, 112, 113, 114, 115, 116, 117 or 118, the complement thereof, or the reverse complement thereof, wherein T may be replaced by U. In a preferred embodiment, the vector as described herein is capable of effecting expression of the polynucleic acid as defined herein, such as the CRISPR as defined herein, or the sequence as set forth in any of SEQ ID NOs: 43, 45, 46, 48, 49, 50, 51, 52, 53, 54, 55, 56, 57, 58-62, 75, 76, 83, or 84.

In a preferred embodiment, the vector as described herein is capable of effecting expression of the polynucleic acid as defined herein, such as the CRISPR as defined herein, or a sequence as comprising a sequence selected from any of SEQ ID NOs: 104, 105, 106, 107, 108, 109, 110, 111, 112, 113, 114, 115, 116, 117 or 118.

To this end, the polynucleic acid sequences as defined herein generally are operably linked to regulatory sequences which permit transcription of said sequence. In other embodiments, the CRISPR as referred to herein comprises, consist of, or consist essentially of a sequence as set forth in any of SEQ ID NOs: 45, 46, 48, 49, 50, 51, 61, 62, 75, 76, 83, 84, 104, 105, 106, 107, 108, 109, 110, 111, 112, 113, 114, 115, 116, 117 or 118, or a sequence having at least 80%, preferably at least 85%, more preferably at least 90%, even more preferably at least 95% sequence identity with a sequence as set forth in any of SEQ ID NOs: 45, 46, 48, 49, 50, 51, 61, 62, 75, 76, 83, 84, 104, 105, 106, 107, 108, 109, 110, 111, 112, 113, 114, 115, 116, 117 or 118, the complement thereof, or the reverse complement thereof, wherein T may be replaced by U. The present invention also relates to a polynucleic acid sequence comprising a CRISPR sequence as defined above, the complement thereof, or the reverse complement thereof, wherein T may be replaced by U. The invention further relates to a polynucleic acid sequence comprising, consisting of, or consisting essentially of a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, or a sequence having at least 80%, preferably at least 85%, more preferably at least 90%, even more preferably at least 95% sequence identity with a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U. In a further aspect, the invention relates to a vector comprising a polynucleic acid sequence as defined above. In another aspect, the invention relates to a polynucleic acid sequence comprising a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, or a sequence having at least 80%, preferably at least 85%, more preferably at least 90%, even more preferably at least 95% sequence identity with a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U. In a preferred embodiment, the vector as described herein is capable of effecting expression of the polynucleic acid as defined herein, such as the CRISPR as defined herein, or the sequence as set forth in any of SEQ ID NOs: 43, 45, 46, 48, 49, 50, 51, 52, 53, 54, 55, 56, 57, 58, 75, 76, 83, 84, 104, 105, 106, 107, 108, 109, 110, 111, 112, 113, 114, 115, 116, 117, 118 or a sequence having at least 80%, preferably at least 85%, more preferably at least 90%, even more preferably at least 95% sequence identity with a sequence as set forth in any of SEQ ID NOs: 43, 45, 46, 48, 49, 50, 51, 52, 53, 54, 55, 56, 57, 58-62, 75, 76, 83, 84 104, 105, 106, 107, 108, 109, 110, 111, 112, 113, 114, 115, 116, 117 or 118. To this end, the polynucleic acid sequences as defined herein generally are operably linked to regulatory sequences which permit transcription of said sequence.

The binding to the genomic DNA is determined by the target sequence in the guide RNA sequence. Herein the length of the target sequence that is used is typically about 20 to 23 nucleotides. Indeed, there is the tendency to use long sequences to assure that a unique sequence in the genome is targeted. However, if a mismatch occurs, the longer the sequence the more chance that the mismatch is tolerated and the wrong target sequence is cut. Thus contrary to common practice, embodiments of guide RNA sequences have a target sequence of 21, 20, 19, 18 or even 17 nucleotides. This shorter length prevents non-specific binding and subsequent erroneous cleavage.

In certain embodiments, more than one dTALEN as referred to herein may be used to target the DMPK locus, e.g. a dTALEN targeting a sequence preceding the DMPK CTG repeats and another dTALEN targeting a sequence after the DMPK CTG repeats, be it either in vitro (e.g. in one of the cell types as described herein elsewhere) or in vivo (i.e. by introduction of the designer nuclease components, optionally together with a homology molecule, as described herein elsewhere).

In certain embodiments, more than one CRISPR as referred to herein may be used to target the DMPK locus, be it either in vitro (e.g. in one of the cell types as described herein elsewhere) or in vivo (i.e. by introduction of the designer nuclease components, optionally together with a homology molecule, as described herein elsewhere). In a preferred embodiment, two CRISPRs as referred to herein are used to target the DMPK locus. In certain embodiments wherein two or at least two CRISPRs are used, one of said CRISPRs comprises, consists of, or consists essentially of a sequence selected from the group comprising or consisting of sequences as set forth in any of SEQ ID NOs: 45, 48, 50, 61, 75, or 76, or a sequence having at least 80%, preferably at least 85%, more preferably at least 90%, even more preferably at least 95% sequence identity with said sequence(s), and another of said CRISPRs comprises, consists of, or consists essentially of a sequence selected from the group comprising or consisting of sequences as set forth in any of SEQ ID NOs: 46, 49, 51, 62, 83, 84, 104, 105, 106, 107, 108, 109, 110, 111, 112, 113, 114, 115, 116, 117 or 118, or a sequence having at least 80%, preferably at least 85%, more preferably at least 90%, even more preferably at least 95% sequence identity with said sequence(s). In certain embodiments, wherein two or at least two CRISPRs are used, one of said CRISPRs comprises, consists of, or consists essentially of a sequence as set forth in SEQ ID NO: 45, or a sequence having at least 80%, preferably at least 85%, more preferably at least 90%, even more preferably at least 95% sequence identity with said sequence, and another of said CRISPRs comprises, consists of, or consists essentially of a sequence as set forth in SEQ ID NO: 46, or a sequence having at least 80%, preferably at least 85%, more preferably at least 90%, even more preferably at least 95% sequence identity with said sequence. In certain embodiments, wherein two or at least two CRISPRs are used, one of said CRISPRs comprises, consists of, or consists essentially of a sequence as set forth in SEQ ID NO: 48, or a sequence having at least 80%, preferably at least 85%, more preferably at least 90%, even more preferably at least 95% sequence identity with said sequence, and another of said CRISPRs comprises, consists of, or consists essentially of a sequence as set forth in SEQ ID NO: 49, or a sequence having at least 80%, preferably at least 85%, more preferably at least 90%, even more preferably at least 95% sequence identity with said sequence. In certain embodiments, wherein two or at least two CRISPRs are used, one of said CRISPRs comprises, consists of, or consists essentially of a sequence as set forth in SEQ ID NO: 50, or a sequence having at least 80%, preferably at least 85%, more preferably at least 90%, even more preferably at least 95% sequence identity with said sequence, and another of said CRISPRs comprises, consists of, or consists essentially of a sequence as set forth in SEQ ID NO: 51, or a sequence having at least 80%, preferably at least 85%, more preferably at least 90%, even more preferably at least 95% sequence identity with said sequence. In certain embodiments, wherein two or at least two CRISPRs are used, one of said CRISPRs comprises, consists of, or consists essentially of a sequence as set forth in SEQ ID NO: 61, or a sequence having at least 80%, preferably at least 85%, more preferably at least 90%, even more preferably at least 95% sequence identity with said sequence, and another of said CRISPRs comprises, consists of, or consists essentially of a sequence as set forth in SEQ ID NO: 62, or a sequence having at least 80%, preferably at least 85%, more preferably at least 90%, even more preferably at least 95% sequence identity with said sequence. In certain embodiments, wherein two or at least two CRISPRs are used, one of said CRISPRs comprises, consists of, or consists essentially of a sequence as set forth in SEQ ID NO: 75 or 76, or a sequence having at least 80%, preferably at least 85%, more preferably at least 90%, even more preferably at least 95% sequence identity with said sequence, and another of said CRISPRs comprises, consists of, or consists essentially of a sequence as set forth in SEQ ID NO: 83 or 84, or a sequence having at least 80%, preferably at least 85%, more preferably at least 90%, even more preferably at least 95% sequence identity with said sequence.

The term “operably linked” as used herein refers to the arrangement of various nucleic acid molecule elements relative to each such that the elements are functionally connected and are able to interact with each other. Such elements may include, without limitation, a promoter, an enhancer and/or a regulatory element, a polyadenylation sequence, one or more introns and/or exons, and a coding sequence of a gene of interest to be expressed (e.g. the dTALEN or CRISPR sequence). The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements.

The term “vector” as used herein refers to nucleic acid molecules, usually double-stranded DNA, which may have inserted into it another nucleic acid molecule, such as a dTALEN or CRISPR sequence. The vector is used to transport the insert nucleic acid molecule into a suitable host cell. A vector may contain the necessary elements that permit transcribing the insert nucleic acid molecule, and, optionally, translating the transcript into a polypeptide. Once in the host cell, the vector may for instance replicate independently of, or coincidental with, the host chromosomal DNA, and several copies of the vector and its inserted nucleic acid molecule may be generated. The term “vector” may thus also be defined as a gene delivery vehicle that facilitates gene transfer into a target cell. This definition includes both non-viral and viral vectors. Non-viral vectors include but are not limited to cationic lipids, liposomes, nanoparticles, PEG, PEI, etc. Viral vectors are derived from viruses including but not limited to: retrovirus, lentivirus, adeno-associated virus, adenovirus, herpesvirus, hepatitis virus or the like. Typically, but not necessarily, viral vectors are replication-deficient as they have lost the ability to propagate in a given cell since viral genes essential for replication have been eliminated from the viral vector.

Preferred vectors are derived from lentivirus, adeno-associated virus, adenovirus, retroviruses and Antiviruses. Alternatively, gene delivery systems can be used to combine viral and non-viral components, such as nanoparticles or virosomes (Yamada et al. (2003) Nat Biotechnol. 21, 885-890).

Retroviruses and Antiviruses are RNA viruses that have the ability to insert their genes into host cell chromosomes after infection. Retroviral and lentiviral vectors have been developed that lack the genes encoding viral proteins, but retain the ability to infect cells and insert their genes into the chromosomes of the target cell (Miller (1990) Mol Cell Biol. 10, 4239-4242; Naldini et al. (1996) Science 272, 263-267; VandenDriessche et al., (1999) Proc Natl Acad Sci USA. 96, 10379-10384. The difference between a lentiviral and a classical Moloney-murine leukemia-virus (MLV) based retroviral vector is that lentiviral vectors can transduce both dividing and non-dividing cells whereas MLV-based retroviral vectors can only transduce dividing cells.

Adenoviral vectors are designed to be administered directly to a living subject. Unlike retroviral vectors, most of the adenoviral vector genomes do not integrate into the chromosome of the host cell. Instead, genes introduced into cells using adenoviral vectors are maintained in the nucleus as an extrachromosomal element (episome) that persists for an extended period of time. Adenoviral vectors will transduce dividing and nondividing cells in many different tissues (Chuah et al. (2003) Blood. 101, 1734-1743). Another viral vector is derived from the herpes simplex virus, a large, double-stranded DNA virus. Recombinant forms of the vaccinia virus, another dsDNA virus, can accommodate large inserts and are generated by homologous recombination.

Adeno-associated virus (AAV) is a small ssDNA virus which infects humans and some other primate species, not known to cause disease and consequently causing only a very mild immune response. AAV can infect both dividing and non-dividing cells and may incorporate its genome into that of the host cell. These features make AAV a very attractive candidate for creating viral vectors for gene therapy, although the cloning capacity of the vector is relatively limited. In a preferred embodiment of the invention, the vector used is therefore derived from adeno associated virus.

As indicated herein elsewhere, a reduction or elimination of the expression of the expanded repeat RNA (CUGexp) of the DMPK gene is effected by introducing in said cells a designer nuclease specifically targeting the DMPK gene.

If the designer nuclease is a dTALEN nuclease, such may for instance be comprised in a vector, as defined herein elsewhere. In case of multiple dTALENs (such as two dTALENs, each comprising a left and a right TALE), both may be present on the same vector or different vectors, and may be introduced in the cells by any means known in the art, such as those defined herein elsewhere, such as by transfection or transduction.

If the designer nuclease is a CRISPR/Cas nuclease, typically both the CRISPR, which may be one or more CRISPR, and the Cas will be introduced in the cell. Both the CRISPR and the Cas may for instance be comprised in a vector, as defined herein elsewhere. Both CRISPR and Cas may be present on the same vector or different vectors, and may be introduced in the cells by any means known in the art, such as those defined herein elsewhere, such as by transfection or transduction.

As used herein, the term “transfection” refers to the introduction of a foreign material like exogenous nucleic acids, typically DNA, into eukaryotic cells by any means of transfer. Different methods of transfection are known in the art and include, but are not limited to, calcium phosphate transfection, electroporation, lipofectamine transfection, DEAE-Dextran transfection, microinjection or virally mediated transfection, i.e. transduction.

As has already been described herein elsewhere, designer nucleases as referred to herein generate double strand breaks in DNA, such as genomic DNA. As is known in the art, generation of double strand breaks typically triggers DNA repair in cells. As a consequence, the cleaved strand is ligated, typically by non-homologous end joining. This process typically or often results in alteration of the repaired DNA strand, compared to the native strand (i.e. the strand prior to cleavage). Deletion or insertion of nucleotides may lead to frame shift mutations, which may result in a defective gene.

If however, besides the designer nuclease also a polynucleic acid sequence is concomitantly introduced, wherein said polynucleic acid sequence is (at least partially) homologous to and bridges the DNA cleavage region, then alternatively to non-homologous end joining of the cleaved DNA, homology-directed DNA repair may take place. This provides a mechanism to delete or replace specifically targeted sequences or reduce or eliminate expression of such sequences (e.g. by introduction of a premature polyadenylation signal).

Accordingly, in certain embodiment, the reduction or elimination of the number of CTG repeats located in the 3′-UTR region of the DMPK gene or the reduction or elimination of the expression of the DMPK gene or the portion of the DMPK gene comprising the CGT repeat region, is effected by homology-directed repair.

By means of example, and without limitation, one or more dTALEN, preferably a combination of left and right TALE, targeting the DMPK CTG repeat may be introduced into a cell as described herein elsewhere, such as the iPS or its progeny, such as the myogenic or neurogenic precursors as defined herein.

By means of another example, and without limitation, one or more CRISPR targeting the DMPK CTG repeat may be introduced into a cell as described, together with a Cas

In preferred embodiments, in addition to the one or more Talen or in addition to the one or more CRISPR and Cas a polynucleic acid sequence is introduced (i.e. a “donor molecule” or “homology sequence” or “donor sequence” or “homology molecule”) having respectively a 5′ and 3′ homology sequence flanking the target site, and wherein preferably the number of CTG repeats is reduced, or wherein expression of the CTG repeat region is reduced or eliminated, or whereby the number or expression of the GTG repeats is reduced or eliminated after homology-directed repair. Preferably, the homology sequence contains less than 50 DMPK CTG repeats, more preferably, the homology sequence contains less than 35 DMPK CTG repeats. In this way cells originally having more than 35, such as more than 50 DMPK CTG repeats may be corrected by homology-directed repair, such that less than 50, preferably less than 35 DMPK CTG repeats remain. Accordingly, in certain embodiments, the reduction or elimination in the cells according to the invention of the number of CTG repeats located in the 3′-UTR region of the DMPK gene to below 50, preferably to below 35 is effected by homology-directed repair, preferably by introducing in said cells a polynucleic acid sequence comprising less than 50, preferably less than 35 CTG repeats. Alternatively, homology-directed repair may be effected by introducing in said cells a polynucleic acid sequence having respectively a 5′ and 3′ homology sequence flanking the target site, and wherein preferably a selectable marker is present inbetween, such as for instance, without limitation, a puromycin expression cassette (i.e. a puromycin under control of a promoter, preferably wherein said promoter is different from the endogenous DMPK promoter, and preferably wherein said promoter is a constitutive promoter or an inducible promoter, which may or may not be tissue-specific). It will be understood that the homology sequence as defined herein may be double stranded or single stranded. In certain preferred embodiments, the donor molecule or homology sequence has a sequence as set forth in SEQ ID NO: 40 or 60 or a variant sequence having at least 70%, preferably at least 80%, more preferably at least 90%, even more preferably at least 95% sequence identity with a sequence as set forth in SEQ ID NO: 40 or 60. It is to be understood that such variant sequence should still be capable of homology-directed repair.

The use of donor molecules in CRIPSR/Cas and TALEN technology has been described. Typically the donor comprises a transgene to knock-in a gene at the nuclease-targeted site. In other applications the donor comprises a selectable marker (e.g. antibiotic resistance gene, fluorescent protein) to verify that recombination of the donor molecule at the nuclease-targeted site has occurred and/or to enrich for gene corrected cells (e.g. by antibiotic selection or fluorescence activated cell sorting).

In the context of the present invention the use of a donor sequence was believed to be of little benefit for the aimed medical application.

The targeted endonuclease activity as envisaged for the purpose of reducing the CTG repeats in the DMPK gene, results in a break in the genome 5′ and 3′ of the expanded CTG repeat. In the absence of the donor the double stranded DNA is repaired by non-homologous end joining (NHEJ), wherein the 5′ strand of the break is degraded by nucleases to create long 3′ single-stranded tails followed by a ligation process. Since this process occurs 3′ of the coding region (i.e. in the non-coding untranslated region), this repair method has little impact on the function of the gene in the excised and repaired gene.

Targeting of the promotor region with NHEJ repair results mainly in insertions or deletions in the gene and premature transcription such that full length RNA comprising the extended CUG repeat is not produced, and accumulation of RNA in nucleofoci does not occur.

In the context of the present invention, it is of importance that the generation of RNA with CUG repeats is inhibited, either by excision of the repeat, either by disruption of the gene. It is thus expected that the efficiency is determined by the efficacy of the nuclease system.

Thus despite the fact that donor molecules are used for insertion (e.g. selectable marker genes) or replacement of genes, the use of donor molecules in knock-out experiments was considered not to influence the efficiency of excising the CTG repeats. In the methods of the present invention wherein the excision of the CTG repeat of the DMPK gene is accompanied by the provision of donor molecules allowing also DNA repair by homologous recombination with the genome flanking the CTG resulted in an unexpected improvement in the reduction of CTG compared to the system relying on NHEJ repair only. Specific types of donor molecule have, not protein encoding cassette no CTG repeats or at most 5, 10, 20, 25 or 50 CTG repeats, such that at most the number of CTG as occurring in a healthy subject are obtained.

To our knowledge, the use of an essentially “empty” donor cassette in combination with a targeted nuclease procedure provides unexpected and surprising advantages.

Donor sequences can also be differentiated base on the following criterion

The donor sequence comprises in the homologous regions 3′ and/or 5′ of the target site in the the sequence of the genome which will be cut by the nuclease, as a consequence both target genomic DNA and donor DNA will be cut by the nuclease.

The regions of homology are 5′ and 3′ of the target site in the genomic DNA, the donor will not contain the sequence of the genomic DNA that is cut by the site specific nuclease.

In certain embodiments, the homology sequence (or donor molecule or homology molecule or donor sequence) as defined herein, comprises, consists of or consists essentially of a polynucleotide sequence as set forth in any of SEQ ID NOs: 7, 8, or 9, the complement thereof, or the reverse complement thereof, wherein T may be replaced by U, or a variant sequence having at least 80%, preferably at least 85%, more preferably at least 90%, even more preferably at least 95% sequence identity with a sequence as set forth in any of SEQ ID NOs: 7, 8, or 9, or a fragment thereof, wherein said fragment preferably comprises at least 50%, more preferably at least 60%, even more preferably at least 70% of the nucleotides of the respective SEQ ID NO or variant sequence, preferably contiguous nucleotides. In certain embodiments, the homology sequence as defined herein comprises, consists of or consists essentially of a polynucleotide sequence as set forth in SEQ ID NO: 8 as well as SEQ ID NO: 9, the complement thereof, or the reverse complement thereof, wherein T may be replaced by U, or a variant sequence having at least 80%, preferably at least 85%, more preferably at least 90%, even more preferably at least 95% sequence identity with a sequence as set forth in respectively SEQ ID NO: 8 or SEQ ID NO: 9, or a fragment thereof, wherein said fragment preferably comprises at least 50%, more preferably at least 60%, even more preferably at least 70% of the nucleotides of the respective SEQ ID NO or variant sequence, preferably contiguous nucleotides. These sequences may flank a number of CTG repeats, preferably less than 50 CTG repeats, more preferably less than 30 CTG repeats, such as for instance 5 CTG repeats.

In certain preferred embodiments, the homology sequences as defined above may be introduced in the cells as defined herein together with any of the dTALEN sequences as defined herein elsewhere. In certain embodiments, a homology sequence as defined herein, comprising, consisting of or consisting essentially of a polynucleotide sequence as set forth in any of SEQ ID NOs: 7, 8, or 9, preferably a sequence comprising both a sequence as set forth in SEQ ID NO: 8 and SEQ ID NO: 9, the complement thereof, or the reverse complement thereof, wherein T may be replaced by U, or a variant sequence having at least 80%, preferably at least 85%, more preferably at least 90%, even more preferably at least 95% sequence identity with a sequence as set forth in any of SEQ ID NOs: 7, 8, or 9, or a fragment thereof, wherein said fragment preferably comprises at least 50%, more preferably at least 60%, even more preferably at least 70% of the nucleotides of the respective SEQ ID NO or variant sequence, preferably contiguous nucleotides, may be introduced into a cell as defined herein elsewhere together with one or more dTALEN encoded by a sequence comprising a sequence as set forth in any of SEQ ID NOs: 1 or 3, preferably both (wherein SEQ ID NO: 1 corresponds to the left TALE and SEQ ID NO: 3 corresponds to the right TALE), or one or more dTALEN having a polypeptide sequence as set forth in any of SEQ ID NO: 2 or 4, preferably both (wherein SEQ ID NO: 2 corresponds to the left TALE and SEQ ID NO: 4 corresponds to the right TALE) or a polynucleic acid sequence encoding a polypeptide sequence as set forth in any of SEQ ID NO: 2 or 4, or a dTALEN capable of binding to a target polynucleic acid sequence as set forth in any of SEQ ID NOs: 5 or 6, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 1 or 3, or SEQ ID NOs: 2 or 4, the complement thereof, or the reverse complement thereof, wherein T may be replaced by U.

In certain embodiments, the homology sequence (or donor molecule or homology molecule or donor sequence) as defined herein, comprises, consists of or consists essentially of a polynucleotide sequence as set forth in any of SEQ ID NOs: 43, 52, 53, 54, 55, 56, 57, 58, or 60, the complement thereof, or the reverse complement thereof, wherein T may be replaced by U, or a variant sequence having at least 80%, preferably at least 85%, more preferably at least 90%, even more preferably at least 95% sequence identity with a sequence as set forth in any of SEQ ID NOs: 1, 43, 52, 53, 54, 55, 56, 57, 58, or 60, or a fragment thereof, wherein said fragment preferably comprises at least 50%, more preferably at least 60%, even more preferably at least 70% of the nucleotides of the respective SEQ ID NO or variant sequence, preferably contiguous nucleotides. In certain embodiments, the homology sequence (or donor molecule) as defined herein comprises, consists of or consists essentially of a polynucleotide sequence as set forth in SEQ ID NO: 54 as well as SEQ ID NO: 55 or 56, the complement thereof, or the reverse complement thereof, wherein T may be replaced by U, or a variant sequence having at least 80%, preferably at least 85%, more preferably at least 90%, even more preferably at least 95% sequence identity with a sequence as set forth in respectively SEQ ID NO: 54 or SEQ ID NO: 55 or 56, or a fragment thereof, wherein said fragment preferably comprises at least 50%, more preferably at least 60%, even more preferably at least 70% of the nucleotides of the respective SEQ ID NO or variant sequence, preferably contiguous nucleotides. These sequences may flank a number of CTG repeats, preferably less than 50 CTG repeats, more preferably less than 30 CTG repeats, such as for instance 5 CTG repeats. SEQ ID NO: 54 corresponds to a DMPK homology sequence flanking the CTG repeat at its 5′ end. SEQ ID NO: 55 and 56 correspond to a DMPK homology sequence flanking the CTG repeat at its 3′ end, wherein SEQ ID NO: 55 is mutated compared to SEQ ID NO: 56 in order to generate an EcoRV restriction site. In other embodiments, the homology sequence as defined herein comprises, consists of or consists essentially of a polynucleotide sequence as set forth in SEQ ID NO: 57 as well as SEQ ID NO: 58, the complement thereof, or the reverse complement thereof, wherein T may be replaced by U, or a variant sequence having at least 80%, preferably at least 85%, more preferably at least 90%, even more preferably at least 95% sequence identity with a sequence as set forth in respectively SEQ ID NO: 57 or SEQ ID NO: 58, or a fragment thereof, wherein said fragment preferably comprises at least 50%, more preferably at least 60%, even more preferably at least 70% of the nucleotides of the respective SEQ ID NO or variant sequence, preferably contiguous nucleotides. SEQ ID NO: 57 corresponds to a DMPK homology sequence flanking the CTG repeat at its 5′ end. SEQ ID NO: 58 corresponds to a DMPK homology sequence flanking the CTG repeat at its 3′ end.

In certain preferred embodiments, the homology sequences as defined above may be introduced in the cells as defined herein together with any of the CRISPR sequences as defined herein elsewhere. In certain embodiments, a homology sequence as defined herein, comprising, consisting of or consisting essentially of a polynucleotide sequence as set forth in any of SEQ ID NOs: 43, 52, 53, 54, 55, 56, 57, 58, or 60, preferably a sequence comprising both a sequence as set forth in SEQ ID NO: 54 and SEQ ID NO: 55 or 56, or a sequence comprising both a sequence as set forth in SEQ ID NO: 57 and SEQ ID NO: 58, the complement thereof, or the reverse complement thereof, wherein T may be replaced by U, or a variant sequence having at least 80%, preferably at least 85%, more preferably at least 90%, even more preferably at least 95% sequence identity with a sequence as set forth in any of SEQ ID NOs: 43, 52, 53, 13, 55, 56, 57, 58, or 60, or a fragment thereof, wherein said fragment preferably comprises at least 50%, more preferably at least 60%, even more preferably at least 70% of the nucleotides of the respective SEQ ID NO or variant sequence, preferably contiguous nucleotides, may be introduced into a cell as defined herein elsewhere together with one or more CRISPR comprising a sequence as set forth in any of SEQ ID NOs: 45, 46, 48, 49, 50, 51, 61, 62, 75, 76, 83, 84, 104, 105, 106, 107, 108, 109, 110, 111, 112, 113, 114, 115, 116, 117 or 118, preferably SEQ ID NO: 45 together with SEQ ID NO: 46, SEQ ID NO: 48 together with SEQ ID NO: 49, SEQ ID NO: 61 together with SEQ ID NO: 62, SEQ ID NO: 50 together with SEQ ID NO: 51, or SEQ ID NO: 75 or 76 together with SEQ ID NO: 83 or 84, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: preferably SEQ ID NO: 45 together with SEQ ID NO: 46, SEQ ID NO: 48 together with SEQ ID NO: 49, SEQ ID NO: 61 together with SEQ ID NO: 62, SEQ ID NO: 50 together with SEQ ID NO: 51 or SEQ ID NO: 75 or 76 together with SEQ ID NO: 83 or 84, the complement thereof, or the reverse complement thereof, wherein T may be replaced by U.

In a further aspect, the invention relates to a pharmaceutical composition comprising the cells as described herein, such as the iPS cells derived from cells originating from a subject having DM1 as described herein, or the progeny thereof, such as precursor cells derived therefrom as described herein, such as myogenic or neurogenic precursors, as described herein elsewhere. Alternatively, such pharmaceutical composition may comprise one or more polynucleic acid sequence as described herein, or one or more vector as described herein. The skilled person will understand that if intended for in vivo gene correction, the polynucleic acid sequences as described herein may be provided in a suitable carrier, as is known in the art, or may be provided in a suitable expression vector (e.g. for in vivo gene delivery). In particular preferred embodiments, such pharmaceutical composition comprises one or more TALEN expression constructs. Optionally, a homology molecule expression construct may be provided in such pharmaceutical composition as well, in order for homology-directed repair to take place. It will be understood that any of the TALENs—or combination of TALENs—as described herein may be provided.

In other particular preferred embodiments, such pharmaceutical composition comprises one or more CRISPR (gRNA) expression constructs as well as a Cas9 expression construct. Optionally, a homology molecule expression construct may be provided in such pharmaceutical composition as well, in order for homology-directed repair to take place. It will be understood that any of the CRISPRs—or combination of CRISPRs—as described herein may be provided.

Particularly preferred delivery vehicles include adeno associated vectors (AAV), as described herein elsewhere. The above pharmaceutical composition may comprise one or more other components besides the cells or polynucleic acid sequences. For example, components may be included that can maintain or enhance the viability of the cells or cell populations. By means of example and without limitation, such components may include salts to ensure substantially isotonic conditions, pH stabilisers such as buffer system(s) (e.g., to ensure substantially neutral pH, such as phosphate or carbonate buffer system), carrier proteins such as for example albumin, media including basal media and/or media supplements, serum or plasma, nutrients, carbohydrate sources, preservatives, stabilisers, anti-oxidants or other materials well known to those skilled in the art.

Also disclosed are methods of producing said compositions by admixing the herein taught cells or cell populations or alternatively the polynucleic acid sequences with one or more additional components as above. The compositions may be for example liquid or may be semi-solid or solid (e.g., may be frozen compositions or may exist as gel or may exist on solid support or scaffold, etc.). Cryopreservatives such as inter alia DMSO are well known in the art. Also disclosed are methods of producing said pharmaceutical compositions by admixing the herein taught cells or cell populations with one or more pharmaceutically acceptable carrier/excipient.

In certain embodiments, the pharmaceutical compositions as described herein may comprise one or more pharmaceutically acceptable carrier/excipient. Preferably, the pharmaceutical compositions may comprise a therapeutically effective amount of the herein taught cells or cell populations, or alternatively the polynucleic acid sequences. The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active substances is well known in the art. Such materials should be non-toxic and should not interfere with the activity of the cells or cell populations. The precise nature of the carrier or excipient or other material will depend on the route of administration. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure iso-osmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, the cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., (1993) Biomaterials 14, 323-330; Mikos et al. (1994) Polymer 35, 1068-1077; Cook et al. (1997) J. Biomed. Mater. Res. 35, 513-523. Such support, scaffold, matrix or material may be biodegradable or non-biodegradable.

In a further aspect, the present invention relates to the cells as described herein, such as the iPS cells derived from cells originating from a subject having DM1 as described herein, or the progeny thereof, such as myogenic or neurogenic precursor cells derived therefrom as described herein, in which cells the expression of an expanded repeat RNA (CUGexp) of the dystrophy myotonic-protein kinase (DMPK) gene is reduced or eliminated, as well as the pharmaceutical composition comprising said cells, as well as the polynucleic acid sequences as described herein. These comprise the polynucleic acid sequences or polypeptide sequences comprising a dTALEN sequence as described herein, such as the dTALEN capable of binding to a target sequence as set forth in any of SEQ ID NOs: 5, 6, or 10-19, or the sequences comprising, consisting of, or consisting essentially of a sequence as set forth in any of SEQ ID NOs: 1 to 19, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 1 to 19, the complement thereof, or the reverse complement thereof, wherein T may be replaced by U; or the vectors comprising said polynucleic acid sequences or polynucleic acid sequences encoding said polypeptide sequences, for use in treating DM1.

These further comprise the polynucleic acid sequences comprising a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U; or the vectors comprising such polynucleic acid sequences, for use in treating DM1.

In certain embodiments, said cells are isolated cells. To this extent, the above referred to cells or sequences may be administered to a subject in need thereof, e.g. a subject having DM1, as defined herein elsewhere. It will be understood that the above referred to cells or sequences may be administered to a subject in need thereof in a therapeutically effective amount. In the alternative, the herein described polynucleic acid sequences, such as the constructs and vectors, may be administered to a subject in need thereof, i.e. a subject having DM1. The skilled person will understand that such in vivo gene therapy may require providing the respective constructs into appropriate delivery vehicles, such as appropriate vectors, by means known in the art. In particular preferred embodiments, one or more TALEN expression constructs may be administered. Any one or more of the herein described TALEN sequences may be administered, preferably a left and right TALEN. In certain embodiments, the dTALEN as referred to herein targets/binds or is capable of targeting/binding to the DMPK promoter. In other embodiments, the dTALEN as referred to herein targets/binds or is capable of targeting/binding to a DMPK enhancer. In other embodiments, the dTALEN as referred to herein targets/binds or is capable of targeting/binding to a DMPK exon. In other embodiments, the dTALEN as referred to herein targets/binds or is capable of targeting/binding to a DMPK intron. In other embodiments, the dTALEN as referred to herein targets/binds or is capable of targeting/binding to a DMPK exon/intron boundary. In other embodiments, the dTALEN as referred to herein targets/binds or is capable of targeting/binding to a DMPK exon encompassing the 5′UTR. In other embodiments, the dTALEN as referred to herein targets/binds or is capable of targeting/binding to a DMPK exon encompassing the 3′UTR. In other embodiments, the dTALEN as referred to herein targets/binds or is capable of targeting/binding to the DMPK CTG repeat. In other embodiments, the dTALEN as referred to herein targets/binds or is capable of targeting/binding to the junction between the DMPK gene sequence and the CTG trinucleotide repeat, preferably the expanded CTG trinucleotide repeat. As used herein, the term “expanded CTG trinucleotide repeat” refers to the CTG trinucleotide repeat having at least 35 CTG trinucleotides, preferably at least 50 CTG trinucleotides. In other embodiments, the dTALEN as referred to herein targets/binds or is capable of targeting/binding to a transcription factor binding site in the DMPK promoter. In other embodiments, the dTALEN as referred to herein targets/binds or is capable of targeting/binding to the AP-2 binding site in the DMPK promoter. In other embodiments, the dTALEN as referred to herein targets/binds or is capable of targeting/binding to the SP1 binding site in the DMPK promoter. In other embodiments, the dTALEN as referred to herein targets/binds or is capable of targeting/binding to the start codon of the DMPK gene. In other embodiments, the dTALEN as referred to herein is encoded by a sequence comprising, consisting of, or consisting essentially of a polynucleic acid sequence as set forth in any of SEQ ID NOs: 1 or 3, preferably both (wherein SEQ ID NO: 1 corresponds to the left TALE and SEQ ID NO: 3 corresponds to the right TALE), the complement thereof, or the reverse complement thereof, wherein T may be replaced by U. The present invention also relates to a polynucleic acid sequence comprising a dTALEN sequence as defined above, the complement thereof, or the reverse complement thereof, wherein T may be replaced by U. The invention further relates to a polynucleic acid sequence comprising, consisting of, or consisting essentially of a sequence as set forth in any of SEQ ID NOs: 1, 3, 5, 6, 7, 8, 9, 10-19, 26, or 27, the complement thereof, or the reverse complement thereof, wherein T may be replaced by U. In another embodiment, the invention relates to a dTALEN as referred to herein comprising, consisting of, or consisting essentially of a polypeptide sequence as set forth in any of SEQ ID NOs: 2 or 4, preferably both (wherein SEQ ID NO: 2 corresponds to the left TALE and SEQ ID NO: 4 corresponds to the right TALE). The present invention also relates to a polypeptide sequence comprising a dTALEN sequence as defined above. The invention further relates to a polypeptide sequence comprising, consisting of, or consisting essentially of a sequence as set forth in any of SEQ ID NOs: 2 or 4. The invention further relates to a dTALEN as referred to herein, comprising, consisting of, or consisting essentially of a polypeptide sequence encoded by a polynucleic acid sequence as set forth in any of SEQ ID NOs: 1 or 3. The skilled person will understand that additional dTALENs (such as combinations of left and right TALEs, or combinations of TALE pairs each composed of a left and right TALE) may be designed which recognize specific additional target sequences, based on the consensus TALE structure and target recognition code (see also FIG. 21). Particularly preferred combinations of left and right TALE target sequences (from which the skilled person can easily design the left and right TALE which recognized these sequences) are listed in Table 2. Particularly suited dTALEN pairs (each comprising a left and right TALEN) according to the present invention are capable of recognizing the TALE target sequence as listed in Table 3.

In alternative particular preferred embodiments, one or more CRISPR expression constructs as well as a Cas9 expression construct may be administered. Any one or more of the above CRISPR sequences may be administered, such as the polynucleic acid sequences comprising a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U.

Optionally, a homology molecule expression construct may be administered as well, in order for homology-directed repair to take place. It will be understood that any of the TALENs—or combination of TALENs—as described herein may be provided.

Alternatively, any of the CRISPRs—or combination of CRISPRs—as described herein may be provided.

Particularly preferred delivery vehicles include adeno associated vectors (AAV), as described herein elsewhere.

Gene therapy protocols, intended to achieve therapeutic gene product expression in target cells, in vitro, but also particularly in vivo, have been extensively described in the art. These include, but are not limited to, intramuscular injection of plasmid DNA (naked or in liposomes), interstitial injection, instillation in airways, application to endothelium, intra-hepatic parenchyme, and intravenous or intra-arterial administration (e.g. intra-hepatic artery, intra-hepatic vein). Various devices have been developed for enhancing the availability of DNA to the target cell. A simple approach is to contact the target cell physically with catheters or implantable materials containing DNA. Another approach is to utilize needle-free, jet injection devices which project a column of liquid directly into the target tissue under high pressure. These delivery paradigms can also be used to deliver viral vectors. Another approach to targeted gene delivery is the use of molecular conjugates, which consist of protein or synthetic ligands to which a nucleic acid- or DNA-binding agent has been attached for the specific targeting of nucleic acids to cells (Cristiano et al. (1993) Proc Natl Acad Sci USA 90, 11548-11552.).

As used herein, the terms “treating” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. As such, “treating” may also encompass amelioration or alleviation of the disease.

As used herein, a phrase such as “a subject in need of treatment” includes subjects, such as mammalian subjects, that would benefit from treatment of a given condition, such as, DM1. Such subjects will typically include, without limitation, those that have been diagnosed with the condition, those prone to have or develop the said condition and/or those in whom the condition is to be prevented.

The term “therapeutically effective amount” refers to an amount of a sequence or pharmaceutical composition of the invention effective to treat a disease or disorder in a subject, i.e., to obtain a desired local or systemic effect and performance. The term thus refers to the quantity of compound or pharmaceutical composition that elicits the biological or medicinal response in a tissue, system, animal, or human that is being sought by a researcher, veterinarian, medical doctor or other clinician, which includes alleviation of the symptoms of the DM1 being treated. In particular, these terms refer to the quantity of sequence or pharmaceutical composition according to the invention which is necessary to prevent, cure, ameliorate, or at least minimize the clinical impairment, symptoms, or complications associated with DM1. in either a single or multiple dose.

In a related aspect, the invention also relates to a method for treating DM1, comprising administering to a subject in need thereof, the cells as described herein, preferably the iPS cells derived from myoblasts or neuronal cells originating from a subject having DM1 as described herein, or the progeny thereof, such as the myogenic or neurogenic precursor cells derived therefrom as described herein, in which cells the expression of an expanded repeat RNA (CUGexp) of the dystrophy myotonic-protein kinase (DMPK) gene is reduced or eliminated, as well as the pharmaceutical composition comprising said cells, as well as the polynucleic acid sequences as described herein, such as the polynucleic acid sequences or the polypeptide sequences comprising a dTALEN sequence as described herein, or the polynucleic acid sequences comprising a sequence as set forth in any of SEQ ID NOs: 1, 3, 5, 6, 7, 8, 9, 10-19, 26, 27, or 40, or the polypeptide sequences as set forth in any of SEQ ID NOs: 2 or 4, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 1, 3, 5, 6, 7, 8, 9, 10-19, 26, 27, or 40, the complement thereof, or the reverse complement thereof, wherein T may be replaced by U; or the polypeptide sequences comprising a sequence as set forth in any of SEQ ID NOs: 2 or 4, or a sequence having at least 80%, preferably at least 85%, more preferably at least 90%, even more preferably at least 95% sequence identity with a sequence as set forth in any of SEQ ID NOs: 2 or 4; or a dTALEN capable of binding to a target sequence as set forth in any of SEQ ID NOs: 5, 6, or 10-25; or the vectors comprising said polynucleic acid sequences or polynucleic acid sequences encoding said polypeptide sequences, preferably in a therapeutically effective amount. In a related aspect, the invention relates to a method for treating DM1, comprising administering to a subject in need thereof one or more of the above TALEN sequences,

In a related aspect, the invention relates to a method for reducing in a cell, preferably in a cell originating from a subject affected with DM1, the number of CTG repeats located in the 3′-UTR region of the DMPK gene or for reducing or eliminating in a cell the expression of an expanded repeat RNA (CUGexp) of the DMPK gene, comprising introducing in said cell a designer nuclease specifically targeting the DMPK gene, as described herein elsewhere, preferably wherein said nuclease comprises a dTALEN, as defined herein. In an embodiment, said method is an in vitro method. In another embodiment, said method is an in vivo method. It will be understood that any one or more of TALEN, preferably a left and right TALEN, optionally a pair of left and right TALEN, as described herein elsewhere, such as the TALENs as defined herein which bind specific target sites, or which comprise the specific polynucleic acid or amino acid sequences as described herein, or encode for specific amino acid sequences as described herein, as well as the specifically disclosed combinations of TALENs (e.g. Tables 2 or 3) may be used.

In a further related aspect, the invention relates to the use of the cells as described herein, preferably the iPS cells derived from myoblasts or neuronal cells originating from a subject having DM1 as described herein, or the progeny thereof, such as myogenic or neurogenic precursor cells derived therefrom as described herein, in which cells the expression of an expanded repeat RNA (CUGexp) of the dystrophy myotonic-protein kinase (DMPK) gene is reduced or eliminated, as well as the pharmaceutical composition comprising said cells, as well as the polynucleic acid sequences or polypeptide sequences as described herein, such as the polynucleic acid sequences or polypeptide sequences comprising a dTALEN sequence as described herein, or the polynucleic acid sequences comprising a sequence as set forth in any of SEQ ID NOs: 1, 3, 5, 6, 7, 8, 9, 10-19, 26, 27, or 40, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 1, 3, 5, 6, 7, 8, 9, 10-19, 26, 27, or 40, the complement thereof, or the reverse complement thereof, wherein T may be replaced by U; or the polypeptide sequences comprising a sequence as set forth in any of SEQ ID NOs: 2 or 4, or a sequence having at least 80%, preferably at least 85%, more preferably at least 90%, even more preferably at least 95% sequence identity with a sequence as set forth in any of SEQ ID NOs: 2 or 4; or a dTALEN capable of binding to a target sequence as set forth in any of SEQ ID NOs: 5, 6, or 10-19; or the vectors comprising such polynucleic acid sequences, for the manufacture of a medicament for treating DM1. In a related aspect, the invention relates to a one or more of the above TALEN sequences for the manufacture of a medicament for treating DM1,

In a related aspect, the invention also relates to a method for treating DM1, comprising administering to a subject in need thereof, the cells as described herein, preferably the iPS cells derived from myoblasts or neuronal cells originating from a subject having DM1 as described herein, or the progeny thereof, such as the myogenic or neurogenic precursor cells derived therefrom as described herein, in which cells the expression of an expanded repeat RNA (CUGexp) of the dystrophy myotonic-protein kinase (DMPK) gene is reduced or eliminated, as well as the pharmaceutical composition comprising said cells, as well as the polynucleic acid sequences as described herein, such as the polynucleic acid sequences comprising a CRISPR sequence as described herein, or the polynucleic acid sequences comprising a sequence as set forth in any of SEQ ID NOs: 143, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 1118, the complement thereof, or the reverse complement thereof, wherein T may be replaced by U; or the vectors comprising such polynucleic acid sequences, preferably in a therapeutically effective amount. In a related aspect, the invention relates to a method for treating DM1, comprising administering to a subject in need thereof one or more of the above CRISPR sequences,

In a related aspect, the invention relates to a method for reducing in a cell, preferably in a cell originating from a subject affected with DM1, the number of CTG repeats located in the 3′-UTR region of the DMPK gene or for reducing or eliminating in a cell the expression of an expanded repeat RNA (CUGexp) of the DMPK gene, comprising introducing in said cell a designer nuclease specifically targeting the DMPK gene, as described herein elsewhere, preferably wherein said nuclease comprises a clustered regulatory interspaced short palindromic repeat (CRISPR)/Cas-based RNA-guided DNA endonuclease. In an embodiment, said method is an in vitro method. In another embodiment, said method is an in vivo method. In an embodiment, said CRIPR comprises a polynucleic acid sequences comprising a sequence as set forth in any of SEQ ID NOs: 143, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U; or the vectors comprising such polynucleic acid sequences.

In a further related aspect, the invention relates to the use of the cells as described herein, preferably the iPS cells derived from myoblasts or neuronal cells originating from a subject having DM1 as described herein, or the progeny thereof, such as myogenic or neurogenic precursor cells derived therefrom as described herein, in which cells the expression of an expanded repeat RNA (CUGexp) of the dystrophy myotonic-protein kinase (DMPK) gene is reduced or eliminated, as well as the pharmaceutical composition comprising said cells, as well as the polynucleic acid sequences as described herein, such as the polynucleic acid sequences comprising a CRISPR sequence as described herein, or the polynucleic acid sequences comprising a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U, or a sequence having at least 80%, preferably at least 85%, more preferably at least 90%, even more preferably at least 95% sequence identity with a sequence as set forth in any of SEQ ID NOs: 43, 45, 46, 48, 49, 50, 51, 52, 53, 54, 55, 56, 57, 58-62, 75, 76, 83, 84, 104, 105, 106, 107, 108, 109, 110, 111, 112, 113, 114, 115, 116, 117 or 118, the complement thereof, or the reverse complement thereof, wherein T may be replaced by U; or the vectors comprising such polynucleic acid sequences, for the manufacture of a medicament for treating DM1. In a related aspect, the invention relates to a one or more of the above CRISPR sequences for the manufacture of a medicament for treating DM1,

In the above treatment methods, the cells or cell populations or alternatively the polynucleic acid sequences may be transplanted or injected to the patient as disclosed elsewhere in this specification, allowing allogeneic, autologous or xenogeneic cellular therapy. For instance, said cells and cell populations may be injected into muscle tissue surgically, by infusion into coronary arteries or delivered with a catheter or they may be injected intravenously.

The tools and methods are intended for use in human therapies. In addition they are also applicable on animal disease models, known in the art.

DM300/SXL mice have an insertion of 45 kb of the DM1 locus, and express the uman DMPK gene with 300 CTG repeats [Seznec (2001) Hum Mol Genet. 10, 2717-2726]. Because of intergenerational instability, the length of the CTG has jumped to more than 1500 repeats in DMSXL mice [Gomes-Pereira M, et al (2007). PLoS Genet. 3, e52]. The expression of mutant DMPK transcripts under the control of the human DMPK promoter is almost ubiquitous and shows pattern similar to that of murine Dmpk transcripts. The human DMPK transgene is expressed at lower levels in skeletal muscle and at higher levels in brain compared with murine Dmpk transcripts and leads to DM1-associated phenotypes, including high mortality, growth retardation, muscle defects, and cognitive impairments

This humanised DM300/SXL mice model contains the human DMPK gene with 300 CTG repeats, such that the tools designed for human applications can be used as such on this model.

All other DM mice model know at present are not generated with human DMPK gene. Mouse specific tools may have to be developed to excise CTG repeats in the genome of these mouse models. Examples thereof include the HSA long repeat (HSA-LR) mice, which express 220 CTG repeats in the 3′ UTR of the human skeletal alpha actin (HSA) gene and the inducible and tissue-specific transgenic EpA960 mice express large interrupted CTG repeats within the DMPK 3′ UTR.

It will be understood that the methods or uses disclosed herein can achieve cell populations comprising or enriched for iPS or the progeny thereof, such as myogenic or neurogenic precursor cells derived therefrom, such as myoblast-like cells or mesoangioblast-like cells, or neuronal/neurogenic cells as described herein elsewhere. For example, a cell population obtained or obtainable according to the methods disclosed herein may comprise at least 40%, preferably at least 50%, more preferably at least 60%, 70%, 80% or more of said cells.

As can be appreciated, the cells as described herein may be further enriched or isolated from cell populations obtained or obtainable according to the methods disclosed herein on the basis of their distinctive characteristics (such as, for example, their marker expression and/or other phenotypic properties taught herein) using methods generally known in the art (e.g., FACS, clonal culture, panning, immunomagnetic cell separation, etc.), thereby yielding isolated cells which are enriched or substantially pure (e.g. at least 85% pure, preferably at least 90% pure, more preferably at least 95% pure or even 99% pure).

In a further aspect, the present invention relates to the use of the cells as described herein, preferably the iPS cells derived from myoblasts or neuronal cells originating from a subject having DM1 as described herein, or the progeny thereof, such as myogenic or neurogenic precursor cells derived therefrom as described herein, optionally in which cells the expression of an expanded repeat RNA (CUGexp) of the dystrophy myotonic-protein kinase (DMPK) gene is reduced or eliminated as an in vitro model for studying DM1 or for drug-screening for identifying therapeutic molecules capable of treating and/or ameliorating DM1. Accordingly, also provided herein are cell-based screening assays, particularly in vitro screening assays, such as, e.g., in assays of biological effects of candidate pharmacological substances and compositions; assays of toxicity of chemical or biological agents; and the like.

Cell-based in vitro screening assays can be carried out as generally known in the art. For example, cells grown in a suitable assay format (e.g., in multi-well plates or on coverslips, etc.) are contacted with a candidate agent (e.g., a potential pharmacological agent) and the effect of said agent on one or more relevant readout parameters is determined and compared to a control. Relevant readout parameters may greatly vary depending on the type of assay and may include, without limitation, survival, occurrence of apoptosis or necrosis, altered morphology, altered responsiveness to external signals or metabolites, gene expression, etc.

As indicated herein elsewhere, the iPS derived from myoblasts or neuronal cells originating from a subject having DM1 as defined herein, as well as the progeny thereof, such as myogenic or neurogenic precursor cells derived therefrom, such as the myopblast-like or mesoangioblast-like cells or neuron-like cells, display a DM1 specific phenotype, in particular nuclear foci, characteristic of accumulated toxic DMPK RNA. Evaluation of such nuclear foci provides a means for studying the disease as well as provides a tool for drug-screening.

Accordingly, in an aspect, the invention relates to a method for identifying therapeutic molecules capable of treating and/or ameliorating DM1, comprising contacting a candidate molecule with the cells as described herein, preferably the iPS cells derived from myoblasts or neuronal cells originating from a subject having DM1 as described herein, or the myogenic or neurogenic precursor cells derived therefrom as described herein. A reduction in the amount, size, or intensity of the nuclear foci is indicative of the candidate molecule being therapeutically effective.

The staining and determination of nuclear foci in iPS and progenitors or precursors thereof provides the evidence that a targeted nuclease methods have an effect on the reduction of CTG repeats in the genome, reduction of CUG repeats in the RNA and reduction of ribonuclear inclusions in the nuclei.

As an initial screening tool, candidate guide RNA's can be tested for their efficacy in IPS cells from DM1 patients, or other viable cells which can be transfected or transduced. The excision of CTG repeats at the genomic level can be measured by PCR techniques or Southern blot analysis. The reduction of CUG repeats in RNA can be determined by RT-PCR. This screening method provides an efficient system for high throughput screening of candidate constructs, and can be followed by a confirmation by iPS nuclear foci staining.

It is to be understood that although particular embodiments, specific constructions and configurations, as well as materials, have been discussed herein for methods and applications according to the present invention, various changes or modifications in form and detail may be made without departing from the scope and spirit of this invention.

The aspects and embodiments of the invention are further supported by the following non-limiting examples.

### Examples

### Example 1: Generation of iPS Cells from DM1 Patient Cells

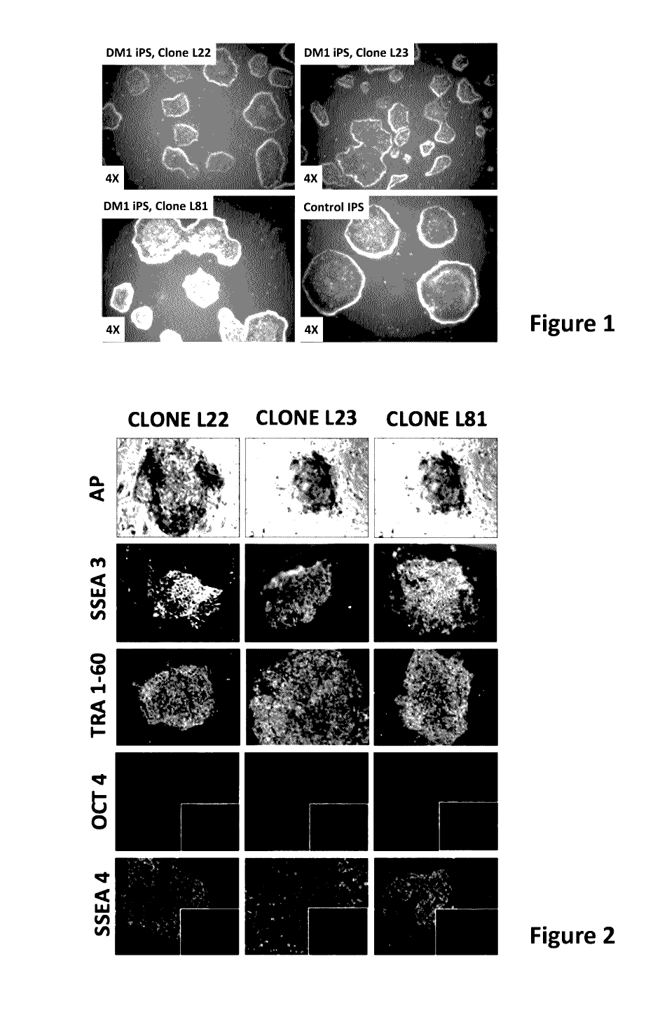

Human iPS cells were derived from myoblast cells from DM1 patients with extensive CTG repeats and from fibroblast of normal donors. For patient details see Table 2. The DM1 iPS cells were generated from skeletal myoblasts obtained from a 46 year old female suffering from DM1 with clinical manifestation of ptosis, slight atrophy, weakness of distal muscle, neckflexors and facial muscles; myotonia; cataract; ECG conduction abnormalities and daytime somnolence. This patient was selected because of the presence of expanded 1250 CTG repeats and because she manifested a severe DM1 phenotype. From a population of DM1 iPS colonies generated from this patient, three distinct iPS clones were selected and isolated for further expansion for subsequent characterizations experiments and for extensive characterization of the DM1 phenotype in differentiated and non-differentiated iPS cells. These expanded clones were designated as L22, L23 & L81, as shown in FIG. 1. Clones were successfully grown in the presence and absence of mouse embryonic fibroblast (MEF).

TABLE 2
Details of iPS cells generated from DM1 patient cells according to
an embodiment of the present invention

| | | Genetic | Cell |
|---|---|---|---|
| Description | Patient symptoms | Defect | Number |
| Myoblast | Age at biopsy: 46 year-old (female) | 1250 | 1 |
| primary | Health status: ptosis; slight atrophy | CTG | million |
| culture: DM1 | and weakness of distal muscles, | repeats | |
| cells with | neckflexors and facial muscles; | | |
| 1250 CTG | myotonia; cataract; ECG conduction | | |
| repeats | abnormalities; daytime somnolence | | |

The method for generating iPS from the DM1 patient myoblast or from normal donor using fibroblast cells was performed using retroviral vectors to deliver the 4 reprogramming factors Oct 4, Sox 2, klf4 and cMyc (OSKM) into these cells.

The protocol for generation of human induced pluripotent stem cells is detailed below.

### 1. Thawing and Passage of Cells

1. The primary human cells used for iPS generation are preferably below passage 7-8.
2. The frozen vials of human cells were thawed at room temperature.
3 The freezing medium containing primary human cells were gently mixed to 8 ml pre-warmed E8 media in a 15 ml tube.
4. This mixture was then centrifuged at 1200 rpm for 3 mins. After centrifugation, the media was aspirated out and cell pellet was resuspended in medium and plated in required culture dish and incubated at 37° C., 5% CO2, 95% humidity.
5. After 24 hours, media change was given with fresh media and kept in culture until confluency is attained.
6. One day prior to transduction, the human cells were Trypsinize and plated for transduction.
7. For trypsinization, the medium was aspirated and washed with DPBS. Add 0.05% Trypsin and incubated at dish at 37° C. for 5 mins.
8. Neutralize trypsin by addition of medium, and transfer the cell suspension to a 15 ml conical tube.
9. Count the cells and plate them at 2×10̂5 cells to per 60 mm dish.

### 2. Day 0: Retroviral Transduction of Human Cells

10. On the day of transduction, 15 ml tube containing 3 ml of pre warmed cells media with retroviral particles for the Oct 4, Sox 2, klf4 and cMyc (OSKM) transcription factors are prepared.
11. The cells are checked under microscope before transduction.
12. Thereafter, the media is aspirated out and fresh media with the OSKM cocktail prepared in step 10 is gently pipetted on to the cells and incubated at 37° C., 5% CO2, 95% humidity.
13. Next day the media is changed with fresh media and plated checked for dead cells due to transduction. If the cell dead is less than 5%, then its normal as expected.
14. The culture dish is observed every day until day 3-4.

### 3. Day 4: Passaging of Transduced Human Cells

15. At around day 4, the cells look confluent and passaged (as mentioned previously in 4.1) into new 60 mm culture dishes at a spilt ratio of 1:3.

### 4. Day 5: Change to hES Media

16. Next day after plating the transduced cells, the medium is replaced with hES media supplemented with VPA (Valproic acid).
17. Media change is provided at every alternative day depending upon the confluency.
18. From day 7-8, the plates are screened regularly for emergence of colony like clusters.

### 5. Day 10-12: Appearance of Colony Like Morphology

19. At around day 11, small colony like morphology starts appearing. Depending on to the sample variation, the time point for appearance of colonies may vary.
20. Thereafter the colonies are microscopically followed every day as they expand in size.
21. Media is regularly changed everyday since the appearance of colony like morphology.

### 6. Day 15: Mechanical Passaging of Colonies

22. At around day 15 when the colonies are suitable to be mechanically passaged, they are manually cut using a sterile 21/22 guage needle and transferred to a new feeder plate.
23. Importantly at this step, each colony is transferred to a single plate containing feeder cells (with small surface area like that of a Organ cell/OC). At this stage each colony is referred to an individual clone and labeled as passaged 0.
24. From this step onwards, during mechanical passaging a small molecule called Thiazovivin™ is added to the hES media at a final concentration of 10 uM. This small molecule helps in attachment and increases the survival of the passaged colonies.
25. Similarly 20-30 clones are mechanically picked up per cell line in subsequent days.

### 7. Regular Initial Mechanical Passaging of Colonies

26. The clones picked up are maintained and passaged separately till passage 10 depending upon their rate of growth and morphology.
27. After passage 10, the clones are expanded and simultaneously frozen.
28. After this stage of expansion of clones, they are characterized for expression of intrinsic pluripotency genes, silencing of the exogenous OSKM factors etc.
29. After characterization of the individual clones, if they are characteristic of iPS cells, they are termed as established iPS cell clones.

The medium for generation and maintainance of iPS on MEF contained: Knock out Serum (Life technologies), Knock out DMEM (Life technologies), Glutamine, NEAA, Penstrep, bFGF, beta mercaptoethanol

On Feeder Free:

Essential 8 medium with supplement from Life technology company
culture on matrix called GELTREX from Life technology company

Immunocytochemistry was performed as follows:

1. Plate about 80-90% confluent 35 mm iPS plate in 6 wells of 24 well plate in feeder free conditions using Essential 8 iPS culture kit (Invitrogen).
2. Change media of the cells every day. After 2 to 3 days, the colonies will reach appropriate size to do immunocytochemistry.
3. Aspirate the media and wash the cells twice with PBS.
4. Fix the cells with 4% paraformaldehyde (PFA) in PBS by incubating 20 min at room temperature (RT).
5. Aspirate 4% PFA in PBS and wash 3 times with PBS during 10 min each time.
6. Block the cells 30 min with Blocking buffer (0.1 g BSA+100 microliter Normal Goat serum, 0.25% Triton-X in PBS). No Triton-X needs to be added for SSEA4 antigen because SSEA4 is a surface antigen.
7. Apply 4 drops (approximately 200 μL) or sufficient volume to cover the cells of Image-iT™ FX signal enhancer (Invitrogen). Incubate for 30 minutes at room temperature in a humid environment.
8. Incubate primary antibody for 2 to 3 hours at RT or overnight at 4° C.; SSEA4 antibody (catalog#-414000, Invitrogen), 5 to 10 microgram per ml; OCT4 antibody (catalog#-13998, Invitrogen), 1:400 dilution.
9. Wash 3 times with PBS during 5 min each time.
10. Add secondary antibody in blocking buffer (1 to 10 microgram per ml). For mouse SSEA4, use Alexa Fluor 488 Goat antimouse SFX kit from molecular probes (#A31619). For Rabbit OCT4, use Alexa fluor 555 goat anti rabbit SFX kit (#A31630).
11. Wash 3 times with PBS.
12. Stain with 300 nM 4′,6-diamidino-2-phenylindole (DAPI) solution in PBS for 1 to 5 min.
13. Visualise under fluorescent microscope.

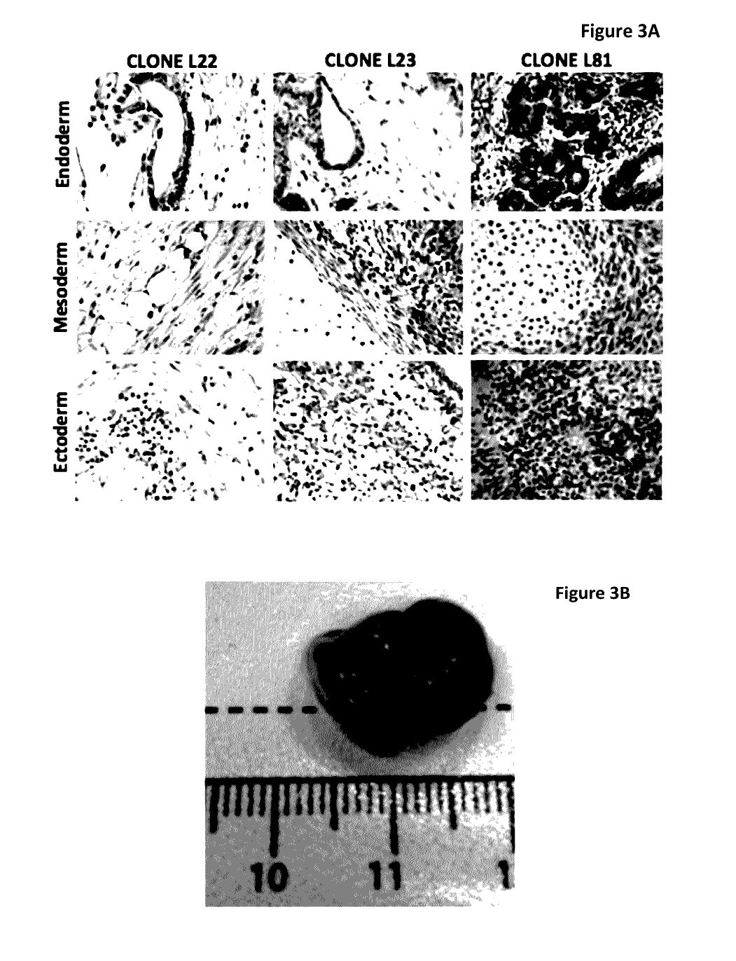

Transfected cells were plated under specific growth condition and colonies with ES-like morphology were picked. The expression of iPS cell markers such as AlkPhos (AP), SSEA-3, SSEA-4, OCT4, and Tra-1-60, was monitored by immunostaining as shown in FIG. 2. FIG. 3 demonstrates the successful generation of teratoma in immunodeficient mice using iPS cells from these DM1 clones. Teratoma formation assay is the gold standard for evaluating the pluripotency of DM1-iPS clones. H&E staining present the three germ layers of the teratoma.

To determine the differentiation ability of the human DM1 iPS cells in vitro, embryoid bodies were induced and expression of endodermal, mesodermal and ectodermal markers was confirmed by RT-PCR or Western blot analysis. To test pluripotency in vivo, DM1 iPS cells were transplanted ectopically into immunodeficient mice (SCID) and teratoma formation was monitored along with histological examination of markers specific for cell types belonging to the three distinct germ layers. The DM1-iPS cells were injected into immuno-compromised mice (CB17-SCID mice) and the tumor formed was dissected after 8 to 12 weeks, once it reached to a size of about 1 to 1.5 centimeter. The dissected tumor tissue was fixed in 4% formalin and embedded in paraffin. The sections of paraffin-embedded tumor tissue were done, followed by hematoxylin and eosin (H&E) staining. The H&E stained sections were visualized under the microscope to detect the tissues for endodermal, mesodermal and ectodermal origin. The three DM1-iPS clones (L22, L23 & L81) showed the presence of tissues derived from the three germ layers i.e. endoderm, mesoderm and ectoderm in the teratoma and therefore confirming the pluripotency of the DM1 iPS clones.

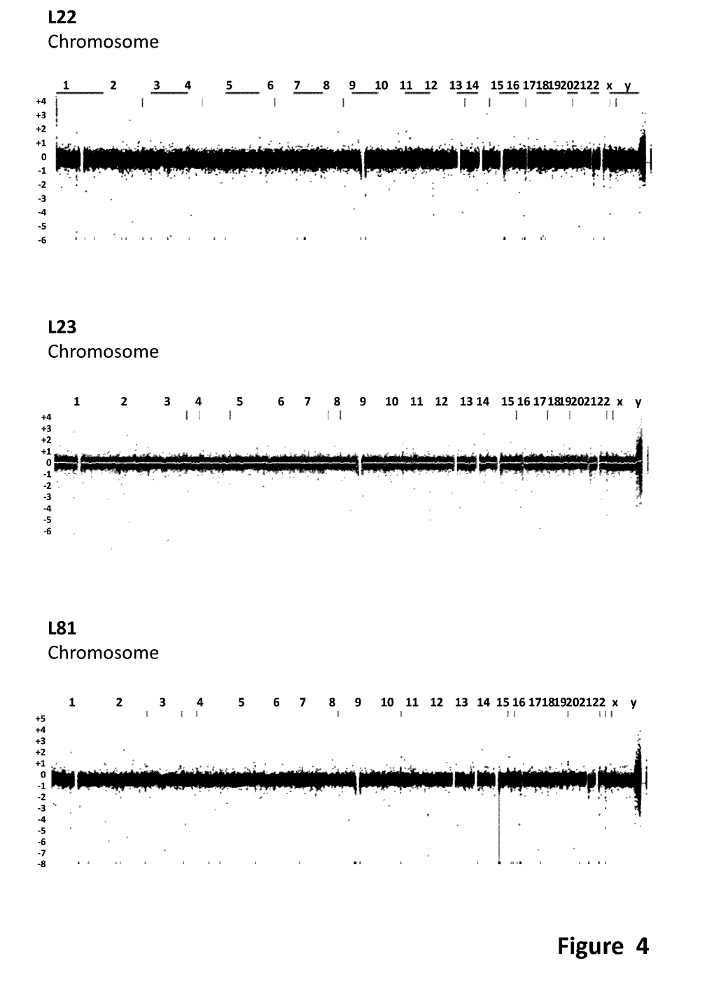

An array comparative genomic hybridization (aCGH) was performed to rule out any gross chromosomal defects in the three DM1-iPS clones. No gross chromosomal abnormalities such as large deletions, insertions or duplications were detected in the three DM1-iPS clones (FIG. 4).

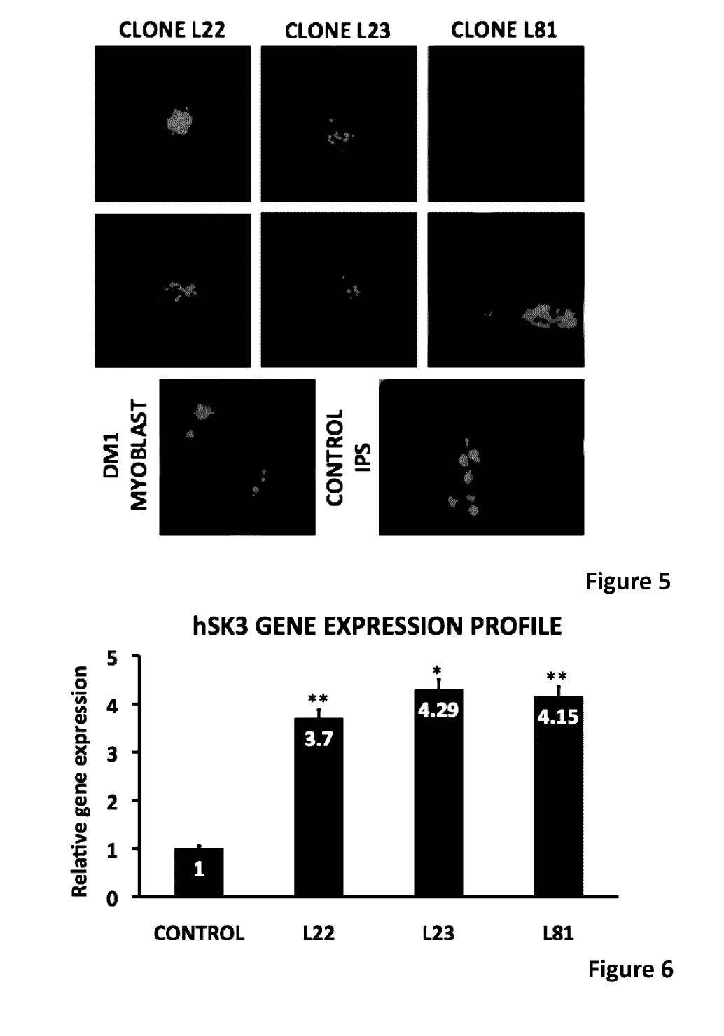

The three DM1-iPS clones showed the presence of nuclear foci on staining with CAG probe (provided by Dr. D. Furling's lab); FIG. 5. These nuclear foci are characteristic of DM1 and are associated with the presence of an expanded CTG repeats. The nuclear foci were clearly visible in DM1 myoblasts from which the three iPS clones were derived, as well as in three DM1-iPS clones, but no nuclear foci were visible in the control iPS, which did not contain expanded CTG repeats. This represents a particularly relevant cellular phenotype of DM1, that can be used as endpoint to assess different therapeutic approaches that are specifically designed to target the pathogenic DM1 RNA. Indeed, if the pathogenic DM1 RNA is inhibited, the phenotypic correction by determining the disappearance of nuclear foci in non-differentiated DM1 iPS cells can be assessed.

Real time quantitative RT-PCR were performed to assessed the expression of several genes that had been reported to be differentially expressed in DM1 such as Ryr, hSK2, hSIX5, Iso A and Iso A B, SK3 and SK1 (Table 1). The control iPS cells were derived from healthy donors and are used as the baseline expression level for comparison. The current data showed a significant increased expression level of SK3 gene by 3 to 4 fold (Table 3 & FIG. 6) in all the three DM1 clones when compared to the control iPS cells. It had been reported that increase in SK3 has a critical role in the increase in Ca2+-induced fragility in DM1 cells (Rhodes et al. (2006) Hum Mol Genet. 15, 3559-3568). Increase in SK3 level is also correlated with myotonia. SK3 expression in muscle was observed to be increased in DM1, ALS as well as polymyositis. All these three diseases have in common the dysfunctioning of the muscle. Therefore SK3 expression seems to be critical for muscle function.

TABLE 3

| | | | | IR | IR | | | |
|---|---|---|---|---|---|---|---|---|
| | Ryr1- | | | Iso | Iso | | | |
| | ASll | hSK2 | hSIX5 | B | A B | hSK1 | hSK3 | SERCA1 |
| L22 | 0.53 | 0.99 | 0.87 | 0.80 | 0.85 | 0.94 | 3.70 | 1.10 |
| L23 | 0.70 | 1.19 | 0.91 | 0.68 | 0.53 | 0.61 | 4.29 | 0.88 |
| L81 | 0.72 | 0.82 | 1.50 | 0.13 | 0.11 | 0.66 | 4.15 | 1.33 |
| control | 1 | 1 | 1 | 1 | 1 | 1 | 1 | 1 |

### Example 2: Coaxed Cardiomyogenic and Myogenic Differentiation of iPS from DM1 Patient Cells

Coaxed myogenic differentiation was induced in human iPS cells derived from cells of normal (healthy) subjects or DM1 patients, making it possible to study the effects of the mutated DMPK gene on myocardial differentiation and functionality.

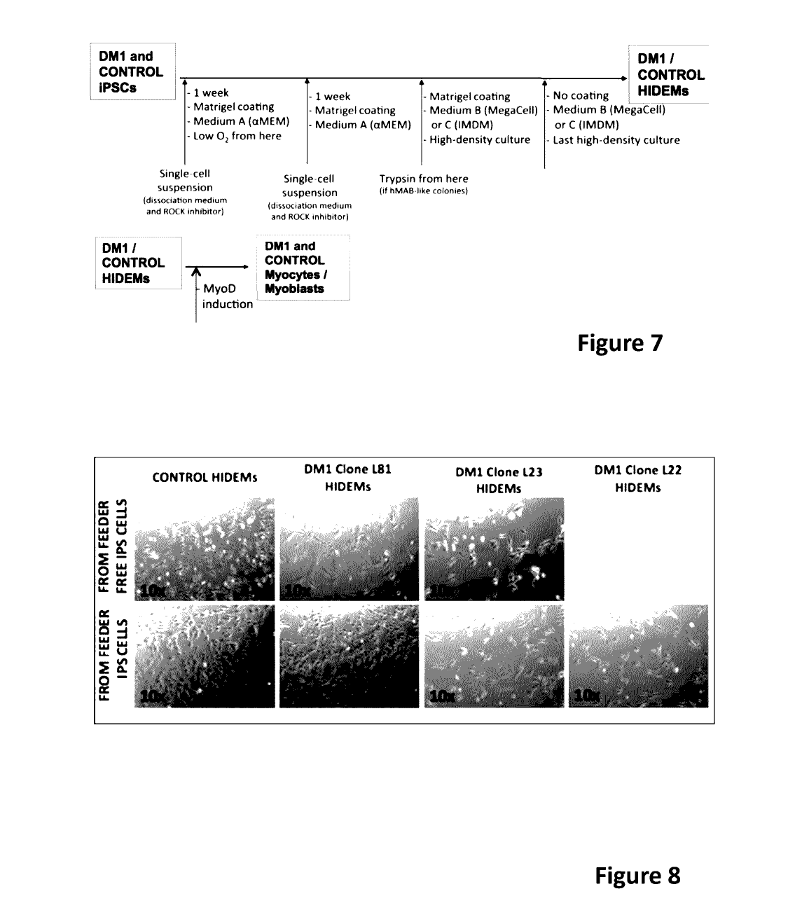

The DM1 iPS clones were expanded and subsequently subjected to myogenic differentiation. For the myogenic differentiation, we follow a 5-step feeder-free differentiation procedure (Tedesco et al. (2012) Sci Transl Med 4, 140ra89); see also FIG. 7. The differentiation protocol was carried out using iPS cells cultured on inactivated feeder cells (inactivated MEF) as per the protocol published by Tedesco et al. (2012). We also used iPS cells cultured on feeder free condition to differentiate by the same protocol. For the clone DM1 L81, DM1 L23 and Control iPS we generated HIDEMs derived from iPS cells cultured both under feeder free and feeder (inactivated MEF) conditions. In case of DM1 L22 clone we have generated from iPS cell, which were cultured on feeder cells. FIG. 8 below shows the morphology of the HIDEMs generated in early passage between p1-p5.

The 5-step differentiation protocol is composed of a 4 stage differentiation protocol to derive HIDEMs from iPSCs and plus final step of HIDEMs differentiation to Mature muscle cells upon MyoD induction. All the 4 stages are of 1 week long and was under Hypoxic condition (3% 02). Firstly, the iPSCs are dissociated into single cell suspension with EDTA based dissociation medium [0.5 mM EDTA, 0.1 mM b-mercaptoethanol, 3% FBS in phosphate-buffered saline (PBS) without Ca2+ and Mg2+] and replated on Matrigel matrix (BD Biosciences) at a density of 6×104 cells/cm2 in a-MEM (Gibco) containing antibiotics (penicillin/streptomycin), 10% FBS, nucleotides, and 0.2% b-mercaptoethanol for 1 week at 37° C., 5% CO2, and 3 to 3% 02. After one week of culture the cells were again dissociated as in step 1 with gentle scraping if required. The cells were replated on Matrigel coated surface at a density of 2.5×104 cells/cm2 in the medium condition as in stage one. In the third stage, the cells are trypsinized and replated on Matrigel at high density (80% confluency) with Mesangioblast (MAB) medium i.e., MegaCell medium (Sigma), containing antibiotics (penicillin/streptomycin), 5% FBS, L-Glutamine, and 0.2% b-mercaptoethanol. In the fourth stage, cells were trypsinized and plated on non Matrigel coated culture surface and cultured in MegaCell medium and passaged as an when confluent. From now on, the cells obtained are maintained like MAB cells. After 4th stage the cells are characterized for markers expression (CD 13, 31, 44, 56, 49b, 45, 146, SSEA4, and AP) (BD Biosciences) by Flow cytometry (BD Biosciences); these cells are also check for Pluripotency markers (hOCT4, hNANOG, hSOX2) and human specific Laminin AC. In the final step of differentiation, these HIDEMs cells obtained were transduced with lentiviral MyoD-ER (MOI-50) and induced with standard Tamoxifen (Sigma) to obtain robust myogenic differentiation.

The media for maintenance of HIDEMs contained: Mega Cell medium from Sigma, FBS from Sigma, Glutamine, NEAA, Penstrep, bFGF, beta marcaptoethanol

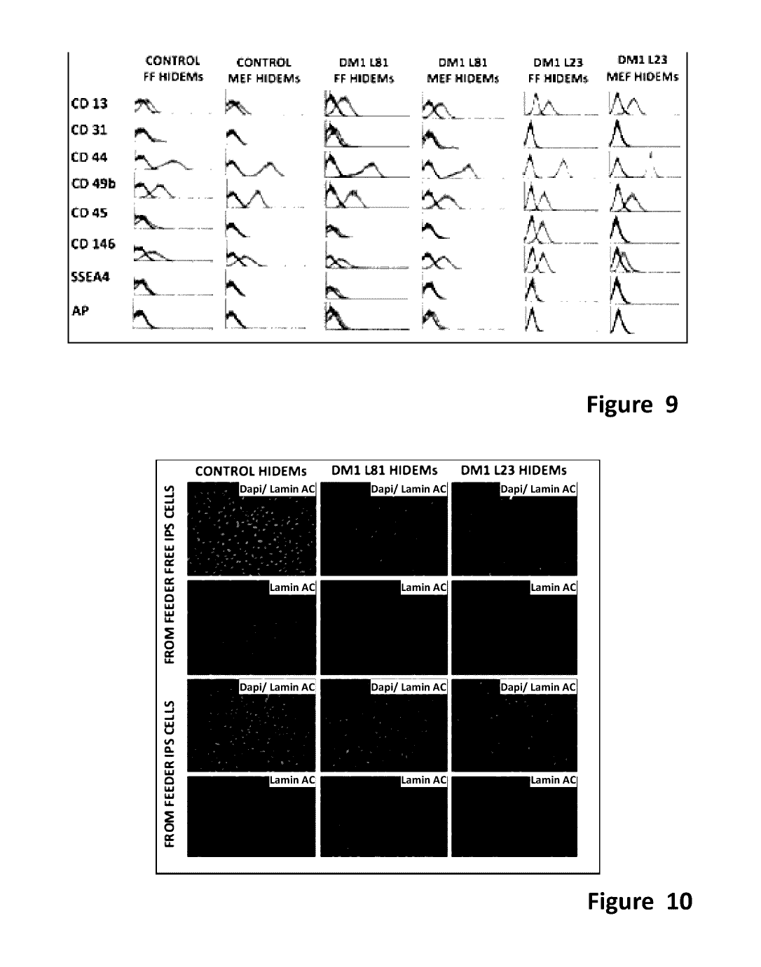

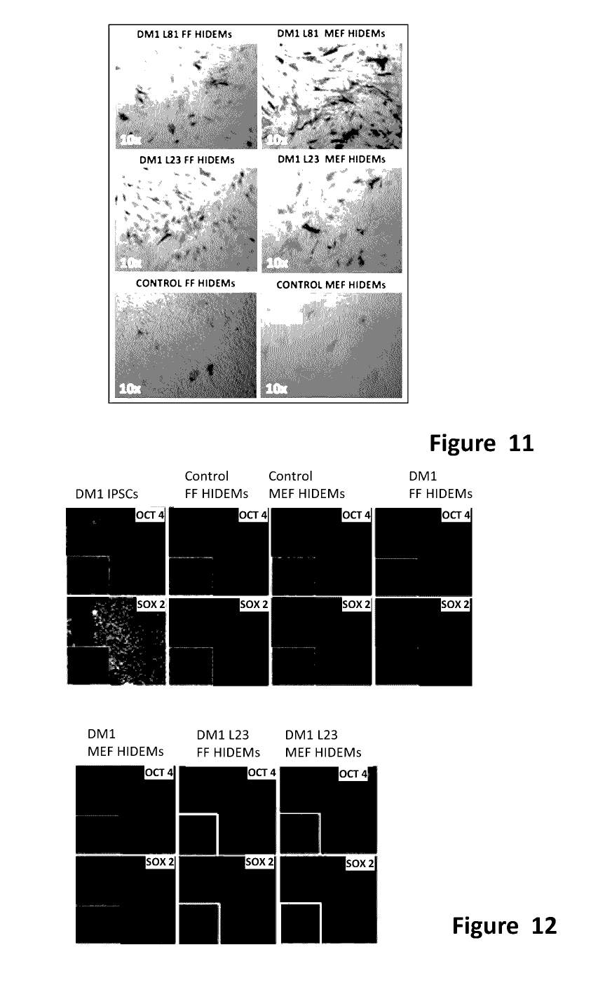

In the process of differentiation, iPS cells gave rise to an intermediate cellular phenotype reminiscent of mesoangioblasts. These human iPSC-derived mesoangioblast-like stem/progenitor cells (designated as HIDEMs) could in turn be induced to differentiate into myoblast-like and myocyte-like cells. The DM1-HIDEMs exhibited a significant increase in expression of CD13, CD44, CD49b and CD146, consistent with a mesoangioblast-like phenotype (FIG. 9). As a part of characterizing the derived HIDEMS, the purity of the HIDEMs cultures was screened. This was tested using a human nuclear specific Lamin AC marker staining which is absent in mouse cells. Analysis of pictures obtained after staining of HIDEMs with Lamin AC showed positive staining for HIDEMs and absence of any lamin AC negative cells (FIG. 10). HIDEMs obtained from feeder free IPS cells, were taken as an internal control. This clearly indicates that there were no carryover MEF feeder cells during the process of differentiation. Moreover, the DM1-HIDEMs also expressed alkaline phosphatase (FIG. 11). Staining for AP was carried out on the 6 HIDEMs lines from Control, DM1 L81 and DM1 L23 iPS clones under both feeder and feeder free conditions. A qualitative analysis of the staining images showed the presence of AP stained cells in the HIDEMs population. Conversely, during the differentiation of iPS cells to HIDEMs, expression of pluripotency markers declined (i.e. hNANOG, hOCT4 and hSOX4) (FIG. 12). In the final step of coaxed differentiation of HIDEMs, the HIDEMs were subjected to MyoD induction after lentiviral transduction. These terminally differentiated cells expressed myosin heavy chain (MyHC) (FIG. 13).

### Example 3: Nuclear Foci Staining Experiment on DM1 L81 iPS Corrected with dTALEN

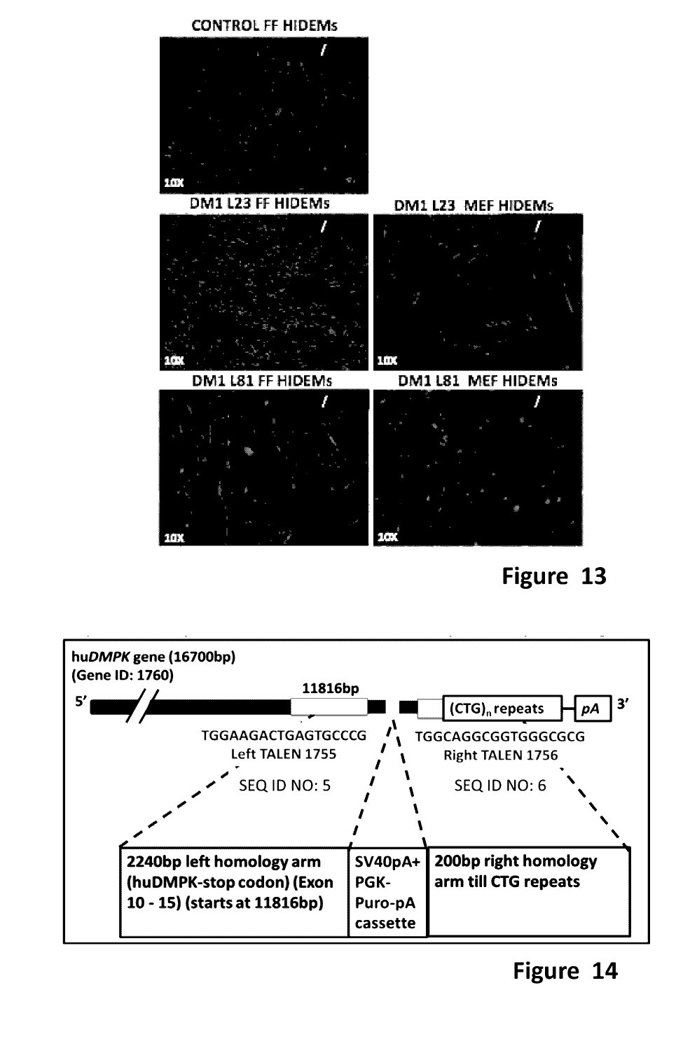

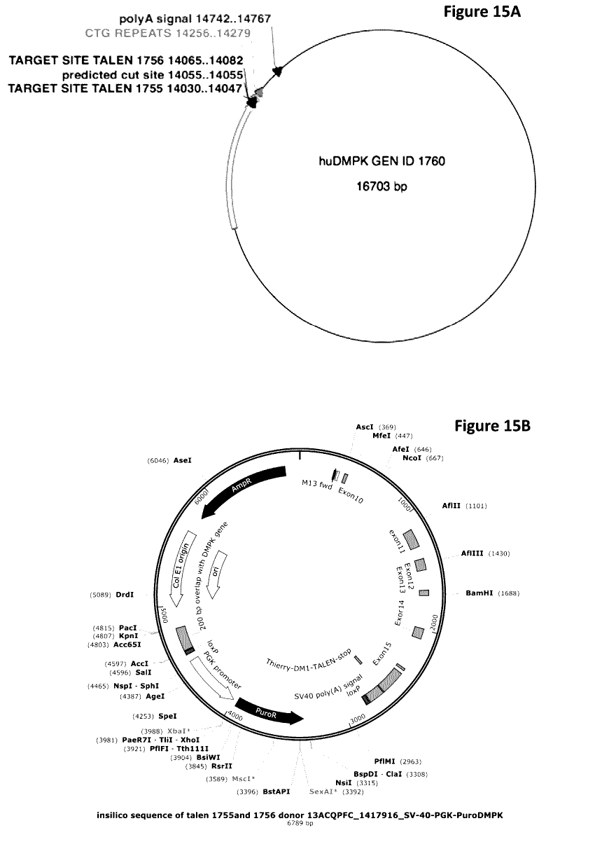

In this experiment, iPS cells derived from DM1 patient were used and iPS cells derived from normal donor were used as control. In order to obtain genetic correction of the expanded CTG repeats in the patient cells, the dTALEN genome-editing tool was used. The dTALEN approach as a ‘molecular scissors’ in combination with a donor molecule was used to specifically target the DMPK gene. Two dTALENs were designed to bind at the appropriately spaced positions of the complementary DMPK strands in order for the FokI to generate a double-strand break in the DMPK gene. A donor molecule (or homology molecule) containing a puromycin expression cassette flanked by left and right homology arms was used for homologous recombination (FIG. 14). The donor molecule incorporated a polyA tail, which prevents transcription of downstream sequences (i.e. the CTG repeats). The donor molecule is as set forth in SEQ ID NO: 7. The left homology arm is as set forth in SEQ ID NO: 8. The right homology arm is as set forth in SEQ ID NO: 9.

One TALEN (“left TALEN 1755”; SEQ ID NO: 1) was designed to bind to nucleotide sequence TGGAAGACTGAGTGCCCG (SEQ ID NO: 5), and another TALEN (“right TALEN 1756”; SEQ ID NO: 3) was designed to bind to nucleotide sequence TGGCAGGCGGTGGGCGCG (SEQ ID NO: 6; which is on the complementary stand of the DMPK gene). The amino acid sequences of the left and right TALEN are set forth in SEQ ID NO: 2 and 4, respectively.

The cloning strategy for designing left TALEN 1755 and right TALEN 1756 is elaborated below.

Cloning Strategy of A626pZ56GFP (A626, Plasmid Nr 41; SEQ ID NO: 26)

The Vector plasmid DR_TAL_1756 was obtained from Keith Joung's lab and restricted with AgeI/BamHI to obtain a 7827 bp vector backbone fragment containing the TALEN 1756 along with the first part of FokI domain. The AgeI/BamHI digestion removes the last part of FokI domain including its STOP codon.

In order to get the insert plasmid, a subcloning step was done in between. For the subcloning we used another TALEN plasmid DR_TAL_1746 procured from Keith Joung's lab, which was digested with HindIII/AgeI to remove the last part of FokI domain including its STOP codon (FRAGMENT 1). Also high fidelity PCR amplification was performed with DR_TAL_1746 as template, Forward Primer GGTGTGATCGTGGATACTAAAGC (FokI region with HindIII site; SEQ ID NO: 28) & Reverse Primer TGGGCCGGGATTCTCCTCCACGTCACCGCATGTTAGA AGACTTCCTCTGCCCTCTCCGCCGCCGGACCTAAAGTTTATCTCGCCGTTATT AAAT (FokI region without STOP codon+newly added 2A sequence in the primer; SEQ ID NO: 29) (FRAGMENT 2). Parallelly another high fidelity PCR amplification was performed with PB-PGK-GFP as template, Forward Primer ATGCGGTGACGTGGAGGAGAATCCCGGCCCAATGCCCGCCATGAAGATCGA G (GFP region+2A peptide part; SEQ ID NO: 30) and Reverse Primer CTCAATGGTGATGGTGATGATGACCGGTTTAGGCGAAGGCGATGGGGGTC (GFP+STOP TALEN; SEQ ID NO: 31) (FRAGMENT 3)(Post gel elution of the amplicon, this fragment was further treated with DpnI enzyme to remove any extra contaminating plasmid used in PCR). Due to the choice of primers, the three fragments (1, 2, 3) had overlapping arms for Gibson assembly, hence they were ligated by Gibson Assembly Kit (Cat# E2611S, NEB).

Therefore we obtained the 1746-2A-GFP plasmid that had a FokI region without STOP codon followed by a 2A-GFP.

The 1331 bp (FokI without STOP codon+2A-GFP) Insert fragment for the main cloning was obtained by digesting the above generated 1746-2A-GFP plasmid with AgeI/BamHI. This was then ligated to the AgeI/BamHI digested vector plasmid DR_TAL_1756 (as explained above).

The good clone was confirmed by sequencing using the following primers:

Reverse primer near the end of FokI domain -

(SEQ ID NO: 32)

CTGACTTCCTCTAAGGTTAAT
Reverse primer downstream of GFP -

(SEQ ID NO: 33)

| GGCAACTAGAAGGCACAGTC | |
|---|---|
| DR_TAL_1756 | Plasmid nr 12 |
| DR_TAL_1746 | Plasmid nr 2 |
| PB-PGK-GFP | |
| 1746-2A-GFP | Plasmid nr 28 |
| A626pZ56GFP | Plasmid nr 41 |

Cloning Strategy of A618pZ55BFP (A618, Plasmid Nr 33; SEQ ID NO: 27)

The Vector plasmid DR_TAL_1755 was obtained from Keith Joung's lab and restricted with AgeI/BamHI to obtain a 7827 bp vector backbone fragment containing the TALEN 1755 along with the first part of FokI domain. The AgeI/BamHI digestion removes the last part of FokI domain including its STOP codon.

In order to get the insert plasmid, a subcloning step was done in between. For the subcloning we used the plasmid 1746-2A-GFP (synthesis explained above). This plasmid was restricted with AgeI/BamHI to obtain a 7827 bp fragment having the plasmid backbone without the last portion of FokI domain and 2A-GFP. Also an overlapping PCR was done with forward primer 1 CCGGCGGATTCCCGAGAGAA (with BamHI site; SEQ ID NO: 34), reverse primer 1 CAGCTCGCTCATTGGGCCGGGATT (SEQ ID NO: 35), template 1 1746-2A-GFP, forward primer 2 CCCGGCCCAATGAGCGAGCTGATT (SEQ ID NO: 36), reverse primer 2 CCCGACCGGTTAATTAAGCTTGTGCCC (SEQ ID NO: 37) and template 2 pCLS9026-CMV-BFP. The primers and template of set 1 amplify a region of FokI domain+2A peptide and those of set 2 amplify the BFP gene from its template. The reverse primer 1 and forward primer 2 are overlapping. Together the PCR produces a FokI-2A-BFP fragment of 1364 bp flanked by AgeI & BamHI sites. This fragment was cloned in the AgeI/BamHI restricted 1746-2A-GFP to get a new plasmid named A612pZ46-2A-BFP.

The 1364 bp Insert fragment was restricted out of A612pZ46-2A-BFP (cloned in lab, explained above) was again digested with AgeI/BamHI and ligated into the AgeI/BamHI digested DR_TAL_1755 plasmid to obtain the final product.

The good clone was confirmed by sequencing using the primers with SEQ ID NO: 38 and 39.

| | |
|---|---|
| DR_TAL_1755 | Plasmid nr 11 |
| 1746-2A-GFP | Plasmid nr 28 |
| A612pZ46-2A-BFP | Plasmid nr 26 |
| pCLS9026-CMV-BFP | |
| A618pZ55BFP | Plasmid nr 33 |

The cloning strategy for the donor molecule (SEQ ID NO: 40) which was used in this experiment (in combination with TALEN 1755 and TALEN 1756) is elaborated below.

The donor molecule used for the TALEN system contained a Pgk-Puro cassette along with an SV40 pA flanked by homology arms on either side. An SV40-PGK-PURO-200bpDMPK fragment was synthesized & cloned by life tech in a company vector backbone. This fragment had the 200 bp right homology arm along with the Pgk-Puro cassette with an in built pA and an SV40 pA (once targeted onto the defective DMPK gene, it would stop the transcription of expanded CTG repeats). This vector plasmid (SV40-PGK-PURO-200bpDMPK) was linearized with NcoI digestion.

For the insert fragment, the 2240 bp left homology arm of the donor molecule was PCR amplified from the genomic DNA of L81 iPS using the forward primer GGCCTAGGCGCGCCATGAGCTCCGCCCTCGG TGTCCCCACAGGATGAAAC (SEQ ID NO: 41) and reverse primer GCAATAAACAAG TTGGGCCATGCCGTGCCCCGGGCACTCAGTCTTCCAAC (SEQ ID NO: 42).

The PCR amplified product had overhangs similar to the NcoI digested SV40-PGK-PURO-200bpDMPK plasmid. Due to the presence of identical overhangs, Gibson assembly could ligate them. Gibson assembly was done using the Gibson assembly Master Mix (Cat # E2611S Bioke, NEB).

The good clones were screened by digestion & confirmed by sequencing.

### SV40-PGK-PURO-DMPK200 bp Plasmid nr 63

In silico sequence of talen 1755 and 1756
donor 13ACQPFC_1417916_SV-40-PGK-PuroDMPK Plasmid nr 64

Additional approaches to target the DMPK locus include among others the deletion or replacement of the CTG repeats by using two flanking TALEN pairs to generate a genomic cut respectively 5′ and 3′ of the CTG repeat region (FIG. 22), and which can be achieved by TALEN pairs recognizing target sequences at set forth in SEQ ID NOs: 10-11 (downstream left and right TALE target respectively) and SEQ ID NOs: 12-13 (upstream left and right TALE target respectively); or the disruption of a critical regulatory region (e.g. the SP1 or AP2 binding site in the DMPK promoter or the DMPK start codon) by using one TALEN pair, of which the cut site overlaps the critical region (FIG. 23). The latter approach can be achieved for targeting the AP2 binding site with a TALE pair recognizing a sequence as set forth in SEQ ID NOs: 14-15 (left and right TALE target respectively), for targeting the start codon with a TALE pair recognizing a sequence as set forth in SEQ ID NOs: 16-17 (left and right TALE target respectively), or for targeting the SP1 binding site with a TALE pair recognizing a sequence as set forth in SEQ ID NOs: 18-19 (left and right TALE target respectively). These and additional target sites are also listed in Table 4.

TABLE 4

| Target name | Target sequence | SEQ ID NO |
|---|---|---|
| 3prime-CTG-Left | TTTCGGCCAGGCTGAGGC | 10 |
| 3prime-CTG-Right | TTCCCAGGCCTGCAGTTT | 11 |
| 5prime-CTG-Left | TCCGAGCGTGGGTCTCCG | 12 |
| 5prime-CTG-Right | TAGGGGGCGGGCCCGGAT | 13 |
| AP2site-Left | TCCAGGGCCTGGACAGG | 14 |
| AP2site-Right | TCGGGGTCCTCCTGTC | 15 |
| atStartcodon-Left | TGGTGCTGCCTGTCCAA | 16 |
| atStartcodon-Right | TGGAGCCGCCTCAGCCG | 17 |
| SP1site-Left | TGTGAGGGGTTAAGGCTG | 18 |
| SP1site-Right | TCCCCACCCCTTGGTCCA | 19 |

Experimental Design

In vitro correction in iPS (protocol I) and HIDEMs (protocol II):

Protocol I: For the in-vitro correction, DM1 iPS, at passage 51 were used for nucleofection using P3 Primary Cell 4D nucleofected X kit (Lonza). Cells at passage 51 were harvested with TrypLE Express (Life technologies), and 2×106 cells were used per nucleofection reaction. The cells were resuspended in 20 μl of nucleofection mixture containing 16.4 μl of P3 Nucleofector solution, 3.6 μl of supplement and required DNA. Thereafter, the reaction mixtures were transferred into a well of Nucleocuvette strips and conducted nucleofection using CB-150 program. Post nucleofection cells were plated in single well of Geltrex (Life technologies) coated 6 well plate in Essential 8 (Life technologies) medium supplemented with ROCK inhibitor and incubated at 37° C., 5% CO2, overnight. Complete media change was provided next day post nucleofection.

Protocol II: For the in-vitro correction, DM1 iPS derived HIDEMs cells, at passage 8 were used for nucleofection using P1 Primary Cell 4D nucleofected X kit (Lonza). Cells at passage 8 were harvested with 0.05% Trypsin EDTA (Life technologies), and 1×106 cells were used per nucleofection reaction. The cells were resuspended in 100 μl of nucleofection mixture containing 80 μl of P1 Nucleofector solution, 20 ml of supplement and required DNA. Thereafter, the reaction mixtures were transferred into a 100 μl Nucleocuvette cuvette and conducted nucleofection using FF104 program. Cells were plated in single well of 6 well plate post nucleofection and incubated at 37° C., 5% CO2, 3% O2 overnight. Complete media change was provided next day post nucleofection.

TABLE 5

| | | NO. OF GFP+/BFP+ |
|---|---|---|
| Conditions | Plasmid Amount | SORTED CELLS |
| Condition 1A (5 reactions) | | 1600 |
| TALEN 1755-BFP | 1 μg | |
| TALEN 1756-GFP | 1 μg | |
| donor molecule | 2 μg | |
| Condition 2A (5 reactions) | | 59700 |
| CMV-BFP | 0.51 μg | |
| CMV-GFP | 0.58 μg | |
| donor molecule | 2 μg | |
| Condition 3A (5 reactions) | | 1900 |
| TALEN 1755-BFP | 1 μg | |
| TALEN 1756-GFP | 1 μg | |
| donor control -dsRED | 1.47 μg | |

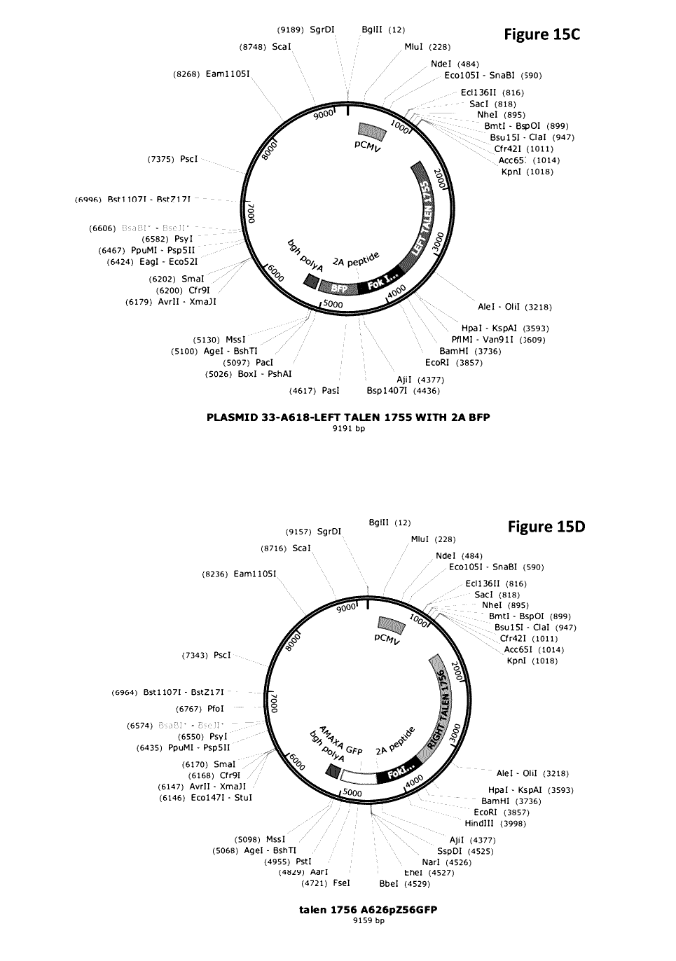

Plasmid maps of the vectors comprising the donor molecule, left TALEN, and right TALEN are illustrated in FIG. 15 A-B (donor molecule), C (left TALEN), and D (right TALEN), respectively.

Post Nucleofection Follow Up

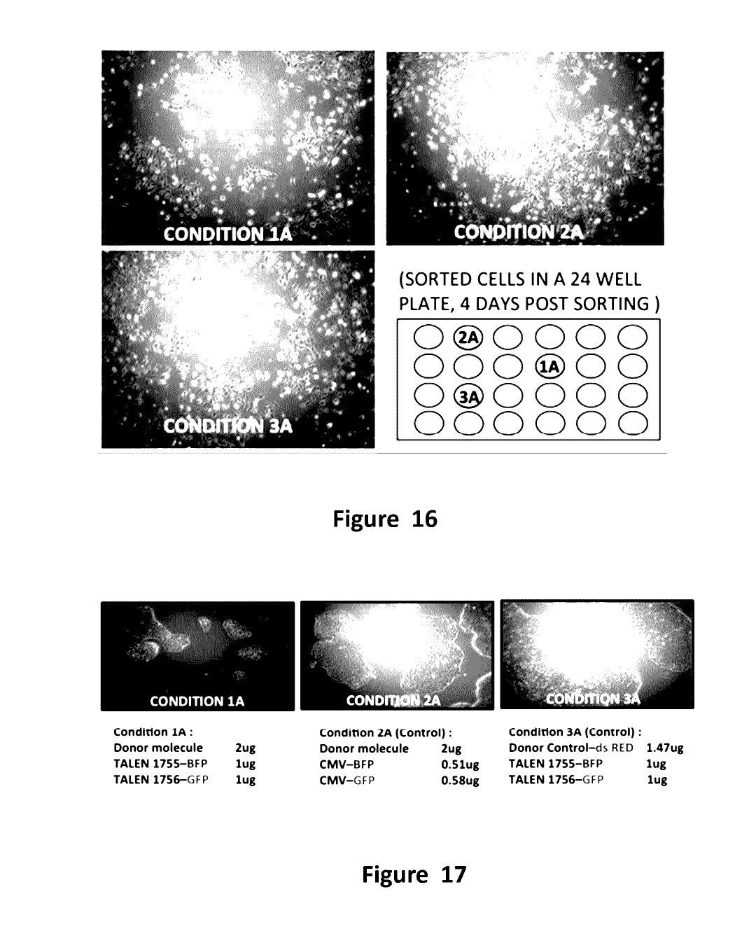

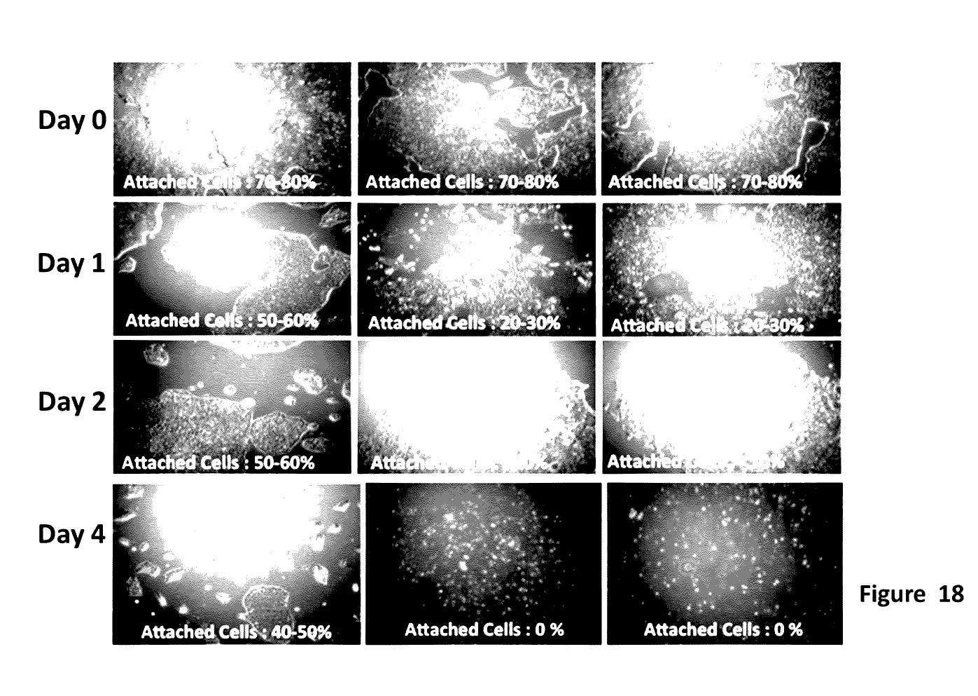

At 48 hours post nucleofection, the nucleofected cells were harvested for cell sorting using FACS Aria III (BD Biosciences). Before harvesting the cells for sorting, qualitative examination of the efficiency of transection was done by microscopic examination of GFP (green fluorescent protein) and BFP (blue fluorescent protein) expression. We sorted out the cells, by selecting the double positive (GFP+BFP+) cell population in our sample. FIG. 16 shows a cell culture 4 days after sorting. FIG. 17 shows a cell culture 14 days after sorting. The sorted cells were expanded and taken for experiments for analysis of dTALEN mediated correction of the DM1 iPS cells by determining the nuclei foci. DM1 cells that do not contain any nuclei foci are corrected cells. After sorting of the cells, the GFP+BFP+ cells were expanded for 18 days until puromycin selection initiation. FIG. 18 shows a cell culture after the indicated days of puromycin selection (conditions 1 A, 2A, and 3A are respectively the bottom, middle, and top row). It is clear that after 4 days of puromycin selection, the number of viable cells in condition 1A is 40-50%, whereas the number of viable cells in control conditions 2A and 3A is 0%. This indicates that homologous recombination between the donor molecule and the targeted region had occurred and the donor molecule containing the puro cassette and the poly A tail had been inserted in the genome of the TALEN targeted cells.

Nuclear Foci Staining of dTALEN Nucleofected and Sorted L22 iPS Cells

For the Nuclear Foci staining, iPS cells were plated at 40,000 cells per 2.4 cm sq (per chamber) of 4-chambered slide (Lab-Tek® II). Next day the cells were used for Nuclear Foci staining.

Materials and Methods

For detecting CUGexpRNA Foci (Nuclear Foci), the cells were fixed with 4% PFA for 15 mins and washed 3 times with 70% ethanol (Sigma Aldrich). Following that two 10 mins wash was given with a solution of PBS and 5 mM MgCl2. The cells were then incubated with PNA-5′Cy3 (CAG)5 3′ (Eurogentec) in Hybridization buffer [2×SSC Buffer (Life technologies), 50% Formamide and 0.2% BSA (Sigma Aldrich)] for 90 mins at 37° C. Post hybridization, the cells were washed with PBS (Life technologies)+0.1% Tween (Sigma Aldrich) for 5 mins. Furthermore it was washed in preheated PBS+0.1% Tween for 30 mins at 45° C. Finally the cells were stained with DAPI nuclear stain (1:500, 5 mins) after a PBS wash. Prior to microscopy the cells were again washed with PBS.

Results

Absence of Nuclear Foci (CUGexpRNA Foci) in dTALEN Corrected iPS Cells

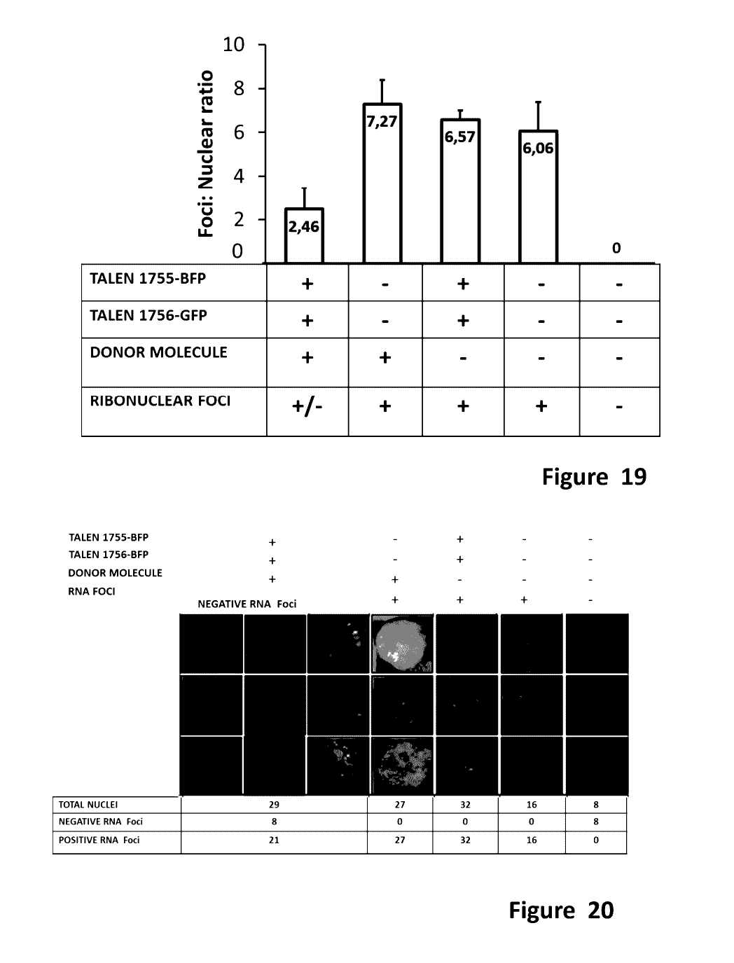

Post CUGexpRNA Foci (Nuclear Foci) staining, microscopic images of the stained nuclei were analyzed per transfection condition. We focused on the number of nuclei with or without Foci. FIGS. 19 and 20 respectively show the foci nuclear ratio and the amount of nuclear foci for the respective conditions. The foci nuclear ratio is determined as the ratio of the total number of ribonuclear foci counted and the total number of nuclei counted. It is clear that the number of foci in condition 1A is less than the control conditions. It was observed that about 30% of the nuclei in the TALEN targeted condition (1A) did not contain RNA foci as compared to control groups where all the nuclei contained RNA foci. This provides evidence for successful incorporation of the puromycin cassette and the polyA STOP before the CTG repeats and subsequent loss of RNA foci in TALEN corrected cells.

### Example 4: Nuclei Foci Staining Experiment on DM1 L81 iPS-Mab Corrected with CRISPR/Cas9 System (ssOligo)

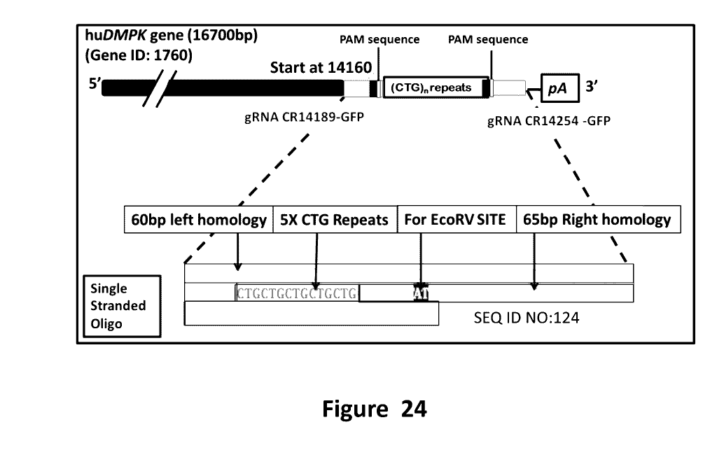

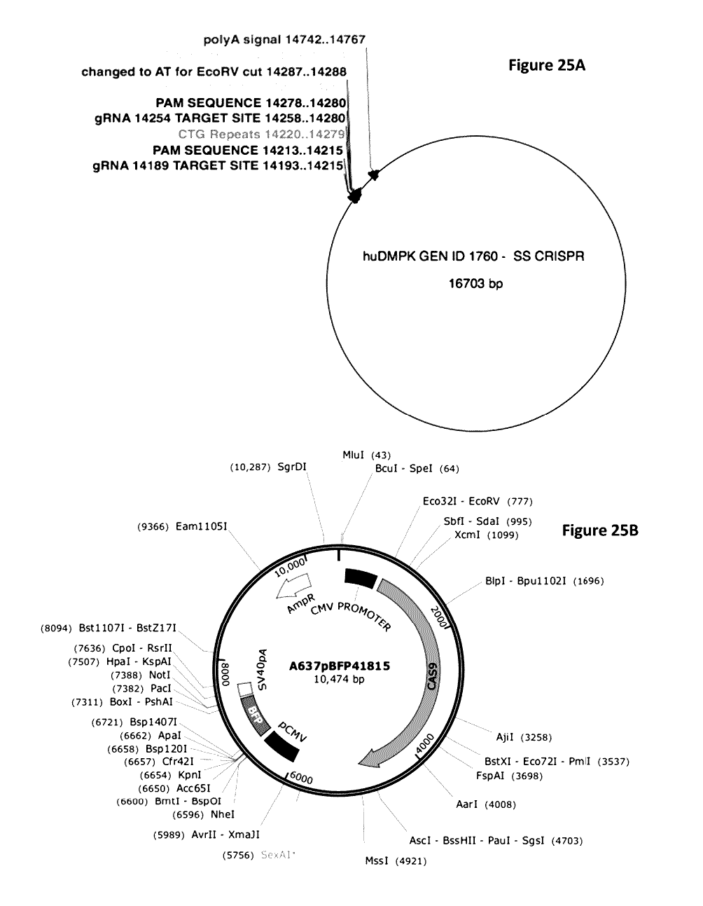

In this experiment, iPS cells derived from DM1 patient were used to obtain a differentiated population of committed muscle precursors cells called HIDEMs. In order to obtain genetic correction of the expanded CTG repeats in the patient cells, the CRISPR/Cas genome-editing tool was used. The RNA-based CRISPR/Cas9 designer nuclease approach as a ‘molecular scissors’ in combination with single-stranded targeting oligo (ssOligo) to specifically excise the expanded CTG repeats of the DMPK gene. Two guide RNAs were designed to cut specifically at the 5′ and 3′ end of the CTG repeats of the DM1 patient iPS-derived HIDEMs. A single stranded oligo designed to contain 5×CTG repeat were used for homologous recombination after the removal of the expanded CTG repeats (FIG. 24).

Experimental Design

The cloning strategy for the Cas9 expression plasmid (SEQ ID NO: 59) as used in this experiment is elaborated below.

The vector plasmid, hCas9 (Cat #41815, addgene), was purchased from addgene and cut open with RsrII/XmajI digestion to obtain an 8827 bp vector fragment, which included the complete Cas9 gene.

The 1647 bp insert fragment, containing CMV-BFP cassette was PCR amplified by forward primer CCCTCCTAGGCCGCCATGCATTAG (with XmajI site; SEQ ID NO: 63) and reverse primer CCCGTTCGGTCCGCGCCTTAAGATACATTG (with RsrII site; SEQ ID NO: 64), using the pCLS9026-CMV-BFP as template. The insert fragment was then digested with RsrII/XmajI and ligated to the vector fragment.

The good clones were confirmed by sequencing for various portions on incorporated BFP gene with the following primers:

Forward Primer upstream pCMV -

(SEQ ID NO: 65)

CCTCTGCCTCTGAGCTATT
Reverse Primer in SV40pA of BFP -

(SEQ ID NO: 66)

GATACCGTAAAGCACGAGGAA

### Cas9 (41815) Plasmid nr 19

pCLS9026-CMV-BFP

### A637pBFP41815 Plasmid nr 45

The cloning strategy for the DM1 gRNA encoding plasmids as used in this experiment is elaborated below.

Guide RNA 14189 (A639pGFP14189-2 plasmid nr 47; SEQ ID NO: 61)

The first step was to create a plasmid (CR14189) having the U6 promoter and the left gRNA 14189-target sequence (specific for our approach). For this, the guide RNA backbone having the U6 promoter (Cat #41824, addgene) was purchased from addgene and cut open with AflII digestion to obtain a 3500 bp fragment. Furthermore, the gRNA 14189 specific for the target site was synthesized by annealing two oligos, mentioned below (the underlined regions indicate identical overhangs in the annealed oligos product with the AflII digested U6 containing backbone)—

(Cr14189_Insert_F; SEQ ID NO: 75)

TTTCTTGGCTTTATATATCTTGTGGAAAGGACGAAACACCGTCGAAGGGT
CCTTGTAGCC

(Cr14189InsertR; SEQ ID NO: 76)

GACTAGCCTTATTTTAACTTGCTATTTCTAGCTCTAAAACGGCTACAAGG
ACCCTTCGAC.

Due to the presence of identical overhangs, Gibson assembly could ligate the U6 containing gRNA backbone and the annealed oligos. Gibson assembly was done using the Gibson assembly Master Mix (Cat # E2611S Bioke, NEB).

The above created plasmid (CR14189) having the U6 promoter and the left gRNA 14189-target sequence was then used as the vector backbone and restricted by DraIII and SfiI to get a 3621 bp vector fragment.

The 1630 bp insert fragment, containing CMV-GFP cassette was PCR amplified by forward primer CCCTGGCCACCATGGCCGCCATGCATTAG (with SfiI site; SEQ ID NO: 77) and reverse primer CCCTCACGAAGTGCGCCTTAAGATACATTG (with DraIII site; SEQ ID NO:78), using the pCLS9025-CMV-GFP as template. The insert fragment was then digested with DraIII/SfiI and ligated to the vector fragment.

The good clones were confirmed by sequencing for various portions with the following primers:

Forward Primer in Kanamycin gene -

(SEQ ID NO: 79)

GGACATAGCGTTGGCTACCC
Reverse Primer downstream the GFP gene -

(SEQ ID NO: 80)

GGTATCTGCGCTCTGCTGAA
Forward Primer in CMV promoter -

(SEQ ID NO: 81)

GTGTACGGTGGGAGGTCTAT
Forward Primer in the backbone upstream of U6 -

(SEQ ID NO: 82)

| CAGGAAACAGCTATGACC | |
|---|---|
| CR41824 | Plasmid nr 25 |
| pCLS9025-CMV-GFP | |
| A639pGFP14189-2 | Plasmid nr 47 |

Guide RNA 14254 (A640pGFP14354-2 Plasmid Nr 48; SEQ ID NO: 62)

The first step was to create a plasmid (CR14254) having the U6 promoter and the right gRNA 14254-target sequence (specific for our approach). For this, the guide RNA backbone having the U6 promoter (Cat #41824, addgene) was purchased from addgene and cut open with Mill digestion to obtain a 3500 bp fragment. Furthermore, the gRNA 14254 specific for the target site was synthesized by annealing two oligos, mentioned below (the underlined regions indicate identical overhangs in the annealed oligos product with the AflII digested U6 containing backbone)—

(Cr14254_Insert_F; SEQ ID NO: 83)

TTTCTTGGCTTTATATATCTTGTGGAAAGGACGAAACACCGCTGCTGCTG
CTGCTGCTGC

(Cr14254InsertR; SEQ ID NO: 84)

GACTAGCCTTATTTTAACTTGCTATTTCTAGCTCTAAAACGCAGCAGCAG
CAGCAGCAGC.

Due to the presence of identical overhangs, Gibson assembly could ligate the U6 containing gRNA backbone and the annealed oligos. Gibson assembly was done using the Gibson assembly Master Mix (Cat # E2611S Bioke, NEB).

The above created plasmid (CR14254) having the U6 promoter and the left gRNA 14254-target sequence was then used as the vector backbone and restricted by DraIII and SfiI to get a 3621 bp vector fragment.

The 1630 bp insert fragment, containing CMV-GFP cassette was PCR amplified by forward primer with SEQ ID NO:85 and reverse primer with SEQ ID NO:86, using the pCLS9025-CMV-GFP as template. The insert fragment was then digested with DraIII/SfiI and ligated to the vector fragment.

The good clones were confirmed by sequencing for various portions with the primers with SEQ ID NO: 79 to 82 as mentioned before.

### CR41824 Plasmid nr 25

pCLS9025-CMV-GFP

### A640pGFP14354-2 Plasmid nr 48

In Vitro Correction in iPS (Protocol I) and HIDEMs (Protocol II):

Protocol I: For the in-vitro correction, DM1 iPS, at passage 51 were used for nucleofection using P3 Primary Cell 4D nucleofected X kit (Lonza). Cells at passage 51 were harvested with TrypLE Express (Life technologies), and 2×106 cells were used per nucleofection reaction. The cells were resuspended in 20 μl of nucleofection mixture containing 16.4 μl of P3 Nucleofector solution, 3.6 μl of supplement and required DNA. Thereafter, the reaction mixtures were transferred into a well of Nucleocuvette strips and conducted nucleofection using CB-150 program. Post nucleofection cells were plated in single well of Geltrex (Life technologies) coated 6 well plate in Essential 8 (Life technologies) medium supplemented with ROCK inhibitor and incubated at 37° C., 5% CO2, overnight. Complete media change was provided next day post nucleofection.

Protocol II: For the in-vitro correction, DM1 iPS derived HIDEMs cells, at passage 8 were used for nucleofection using P1 Primary Cell 4D nucleofected X kit (Lonza). Cells at passage 8 were harvested with 0.05% Trypsin EDTA (Life technologies), and 1×106 cells were used per nucleofection reaction. The cells were resuspended in 100 μl of nucleofection mixture containing 80 μl of P1 Nucleofector solution, 20 ml of supplement and required DNA. Thereafter, the reaction mixtures were transferred into a 100 μl Nucleocuvette cuvette and conducted nucleofection using FF104 program. Cells were plated in single well of 6 well plate post nucleofection and incubated at 37° C., 5% CO2, 3% 02 overnight. Complete media change was provided next day post nucleofection.

Two different amounts of DNAs were used for the experiment; SET A: containing 3 μg of Cas9 plasmid, 3 μg of gRNA CR14189 plasmid, 3 μg of gRNA CR14254 plasmid and 250 pmoles of ssOligo. Whereas SET B contained double the amount of each respective plasmids with same amount of ssOligo. Control conditions without Cas9 plasmid but replaced by a CMV-BFP plasmid as control were used (as in Table 6).

TABLE 6
Cell line: DM1 HIDEMs clone L81
Program: FF-104

| | | |
|---|---|---|
| Conditions | Plasmid Amount | Number of Reaction |

SET A

| | | | |
|---|---|---|---|
| Condition 1A | | | 2 nucleofection reactions |
| Cas9-BFP | 3 | μg | |
| gRNA CR14189-GFP | 3 | μg | |
| gRNA CR14254-GFP | 3 | μg | |
| ssOligo | 250 | pmoles | |
| Condition 2A | | | 2 nucleofection reactions |
| CMV-BFP | 1.36 | μg | |
| gRNA CR14189-GFP | 3 | μg | |
| gRNA CR14254-GFP | 3 | μg | |
| ssOligo | 250 | pmoles | |

SET B

| | | | |
|---|---|---|---|
| Condition 3B | | | 2 nucleofection reactions |
| Cas9-BFP | 6 | μg | |
| gRNA CR14189-GFP | 6 | μg | |
| gRNA CR14254-GFP | 6 | μg | |
| ssOligo | 250 | pmoles | |
| Condition 4B | | | 2 nucleofection reactions |
| CMV-BFP | 2.72 | μg | |
| gRNA CR14189-GFP | 6 | μg | |
| gRNA CR14254-GFP | 6 | μg | |
| ssOligo | 250 | pmoles | |

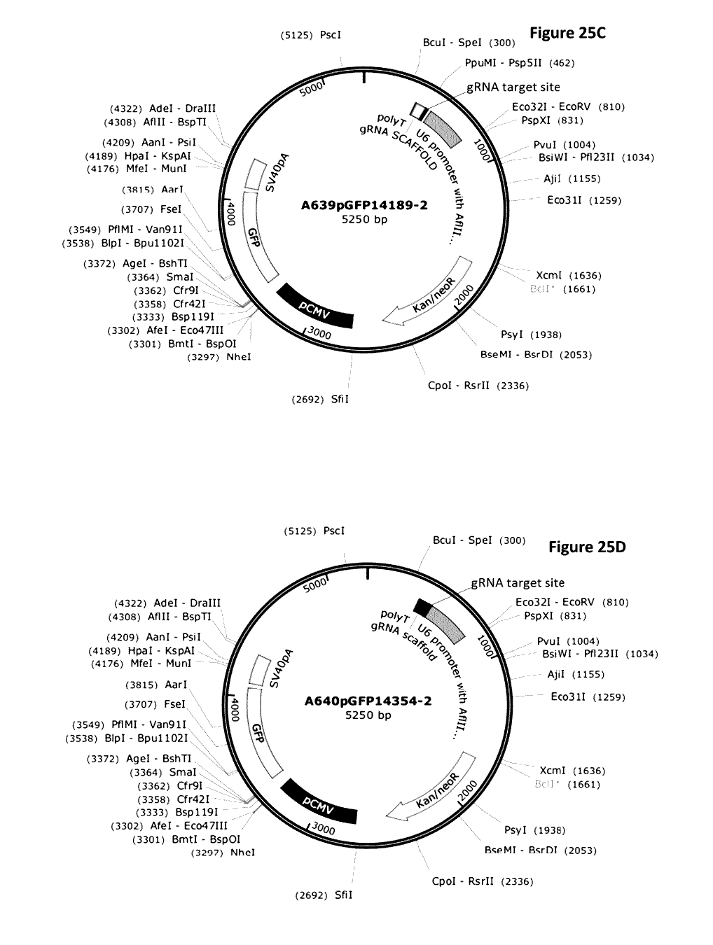

Plasmid maps of the vectors comprising the ssOligo, Cas9-BFP, gRNA CR14189, and gRNA CR14254 are illustrated in FIGS. 25 A, B, C, and D, respectively. The nucleotide sequence of the ssOligo, Cas9, gRNA CR14189, and gRNA CR14254 corresponds to SEQ ID NOs: 43 to 46, respectively. In SEQ ID NO: 43, nucleotides 1-60 correspond to the left homology arm, nucleotides 61 to 75 correspond to 5 CTG repeats, and nucleotides 76 to 140 correspond to the right homology arm. Nucleotides 83-84 (“AT”) replace the corresponding nucleotides “CA” of the native DMPK gene (i.e. nucleotides 416-417 of SEQ ID NO: 47) in order to generate an EcoRV restriction site. In SEQ ID NO: 45, nucleotides 1-19 correspond to the gRNA target site, and nucleotides 20-96 correspond to the gRNA scaffold. In SEQ ID NO: 46, nucleotides 1-20 correspond to the gRNA target site, and nucleotides 21-97 correspond to the gRNA scaffold. SEQ ID NOs: 48 and 49, respectively correspond to SEQ ID NOs: 45 and 46, wherein the gRNA corresponding sequence is fused to the U6 promoter and a poly-T. SEQ ID NOs: 50 and 51, respectively correspond to the target site of SEQ ID NOs: 45 and 46.

Post Nucleofection Follow Up

At 48 hours post nucleofection, the nucleofected cells were harvested for cell sorting using FACS Aria III (BD Biosciences). Before harvesting the cells for sorting, qualitative examination of the efficiency of transfection was done by microscopic examination of GFP (green fluorescent protein) and BFP (blue fluorescent protein) expression. We sorted out the cells, which contained Cas9 with one or both the gRNAs by selecting the double positive (GFP+BFP+) cell population in our sample. We could obtain 54%, 70%, 76% and 53% double positive (GFP+BFP+) cells for condition 1A, 2A, 3B and 4B respectively (see also Table 7). The sorted cells were expanded and taken for experiments for analysis of CRISPR/Cas mediated correction of the DM1 HIDEMs cells by determining the nuclei foci. DM1 cells that do not contain any nuclei foci are corrected cells.

TABLE 7

| | | | | NO. OF |
|---|---|---|---|---|
| | | | | GFP+/BFP+ |
| | % GFP | % BFP | % GFP+ | SORTED |
| CONDITIONS | TOTAL | TOTAL | BFP+ | CELLS |
| 1A | 70% | 54% | 54% | 267,500 |
| 2A | 73% | 72% | 70% | 310,000 |
| 3B | 78% | 82% | 76% | 345,000 |
| 4B | 72% | 89% | 53% | 255,000 |

Nuclear Foci Staining of CRISPR/Cas Transfected and Sorted HIDEMs Cells

For the Nuclear Foci staining, HIDEMs cells were plated at 40,000 cells per 2.4 cm sq (per chamber) of 4-chambered slide (Lab-Tek® II). Next day the cells were used for Nuclear Foci staining.

Materials and Methods

For detecting CUGexpRNA Foci (Nuclear Foci), the cells were fixed with 4% PFA for 15 mins and washed 3 times with 70% ethanol (Sigma Aldrich). Following that two 10 mins wash was given with a solution of PBS and 5 mM MgCl2. The cells were then incubated with PNA-5′Cy3 (CAG)5 3′ (Eurogentec) in Hybridization buffer [2×SSC Buffer (Life technologies), 50% Formamide and 0.2% BSA (Sigma Aldrich)] for 90 mins at 37° C. Post hybridization, the cells were washed with PBS (Life technologies)+0.1% Tween (Sigma Aldrich) for 5 mins. Furthermore it was washed in preheated PBS+0.1% Tween for 30 mins at 45° C. Finally the cells were stained with DAPI nuclear stain (1:500, 5 mins) after a PBS wash. Prior to microscopy the cells were again washed with PBS.

Results

Absence of Nuclear Foci (CUGexpRNA Foci) in CRISPR/Cas Corrected HIDEMs Cells

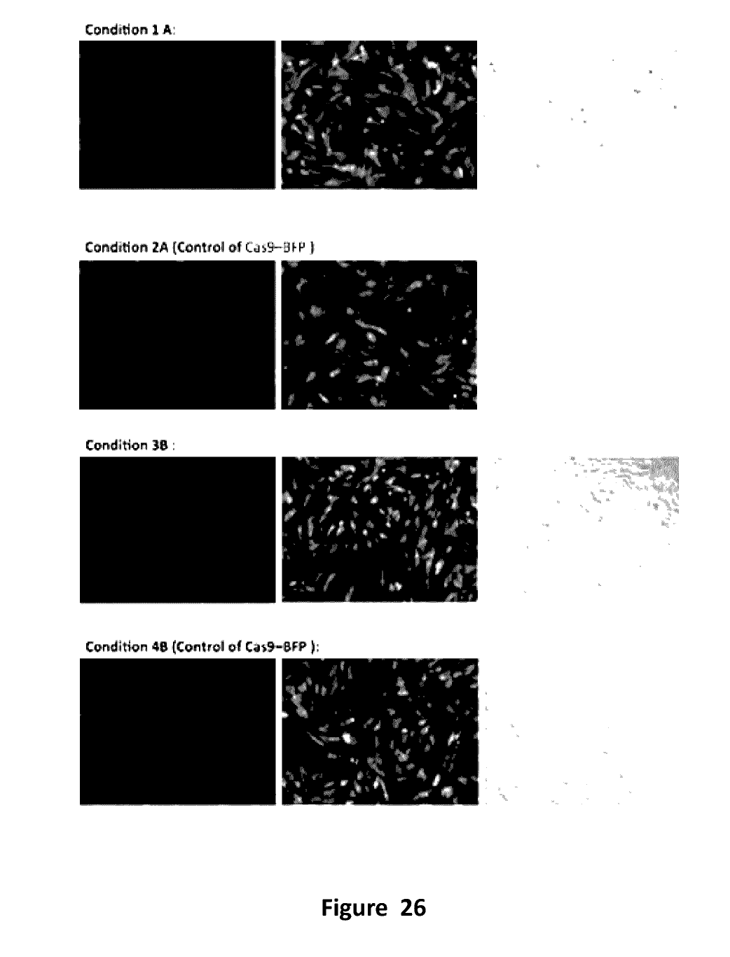

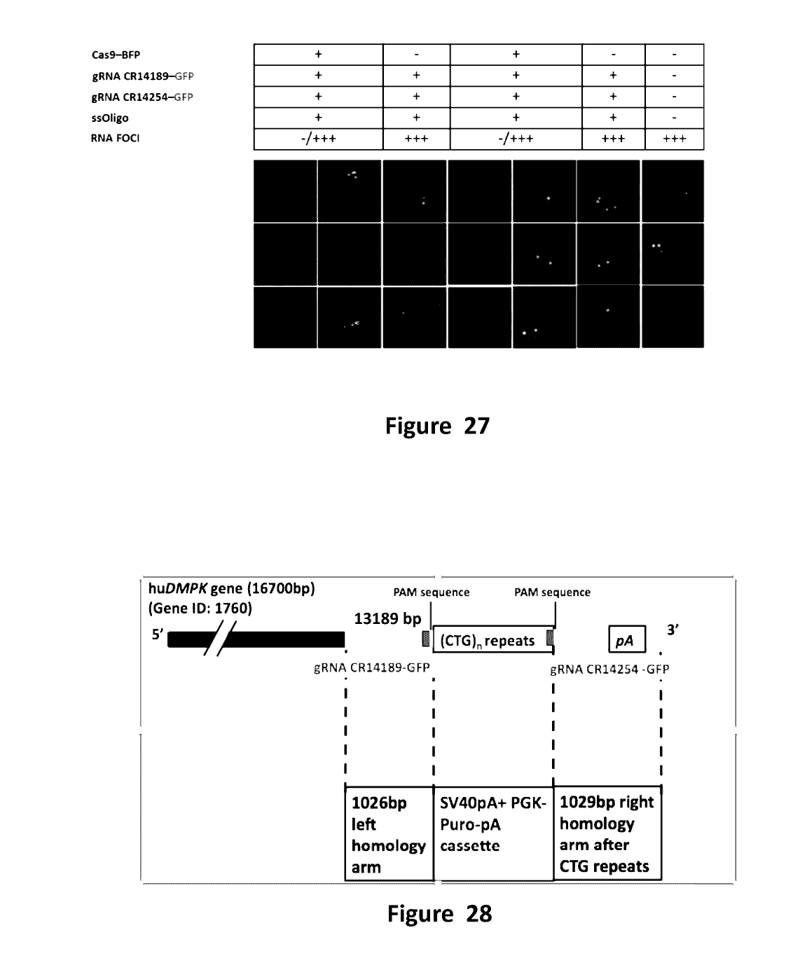

Post CUGexpRNA Foci (Nuclear Foci) staining, microscopic images of the stained nuclei were analyzed for an average of 240 nuclei per transfection condition. We focused on the number of nuclei with or without Foci. We could obtain 4 nuclei without any RNA foci in the CRISPR/Cas transfected conditions as compared to the control conditions, consistent in both sets with low and higher amount of DNA; FIG. 26 (overview) and 27 (individual cells).

In conclusion, the results show the first report demonstrating correction of DM1 patient iPS derived muscle precursors by CRISPR/Cas system. In both condition 1 & 3, where the HIDEMs cells has been transfected with Cas9 plasmid, there are presence of corrected cells demonstrated by Nuclear Foci free nucleus. The efficiency of correction is about 1.7% and 1.5% with 3 ug and 6 ug Cas9 respectively, calculated as follows: 4 Foci free nuclei divide by 235 total nuclei counted=1.7% & 4/273=1.5%.

TABLE 8

Total Nuclei Nr

| | 235 | 214 | 273 | 238 |
|---|---|---|---|---|
| Total Foci | 816 | 605 | 781 | 784 |
| Nr of Foci free | 4 | 0 | 4 | 0 |
| Nuclei | | | | |

The 2^{nd}, 3^{rd}, 4^{th} and 5^{th} columns correspond to Condition 1A, 2A, 3B and 4B of this experiment (Table 8 above).

### Example 5: Nuclei Foci Staining Experiment on DM1 L81 iPS Corrected with Crispr Cas System (Donor Molecule)

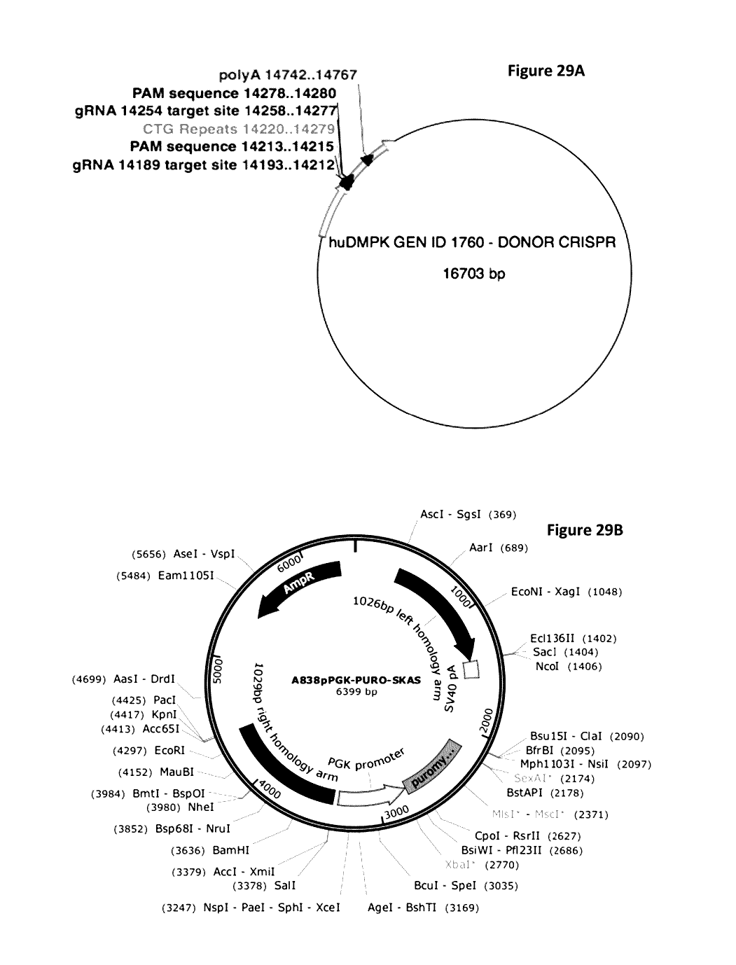

A similar experiment as Example 3 was performed. The correction was done however on iPS cells, and instead of a ssOligo, a donor molecule was co-delivered with the Cas9 and gRNA constructs (see FIG. 28). The donor molecule contained a puromycin selection marker flanked by left and right homology arms (see FIG. 29).

The nucleotide sequence of the Cas9, gRNA CR14189, and gRNA CR14254 corresponds to SEQ ID NOs: 44 to 46, respectively. SEQ ID NO: 52 corresponds to the nucleotide sequence of the donor molecule containing the puromycin expression cassette flanked by left and right homology arms. In SEQ ID NO: 52, nucleotides 1-1026 correspond to the left homology arm, nucleotides 1027 to 1172 correspond to SV40 pA, nucleotides 1173 to 1772 correspond to the puromycin, nucleotides to 1773 to 2368 correspond to the PGK promoter, and nucleotides 2369 to 3397 correspond to the right homology arm. In SEQ ID NO: 45, nucleotides 1-19 correspond to the gRNA target site, and nucleotides 20-96 correspond to the gRNA scaffold. In SEQ ID NO: 46, nucleotides 1-20 correspond to the gRNA target site, and nucleotides 21-97 correspond to the gRNA scaffold. SEQ ID NOs: 48 and 49, respectively correspond to SEQ ID NOs: 45 and 46, wherein the gRNA corresponding sequence is fused to the U6 promoter and a poly-T. SEQ ID NOs: 50 and 51, respectively correspond to the target site of SEQ ID NOs: 45 and 46.

The cloning strategy for the donor molecule (SEQ ID NO: 60) as used in this experiment is elaborated below.

The donor molecule used for the CRISPR/Cas system contained a Pgk-Puro cassette along with an SV40 pA. The Pgk-Puro+SV40 pA segment was taken from the SV40-PGK-PURO-DMPK200 bp plasmid, which was synthesized and used for TALEN-Donor cloning (details in TALEN—donor cloning). This plasmid when digested with KpnI/SalI, gave us a fragment of 4342 bp which contained the Pgk-Puro+SV40 pA segment. This KpnI/SalI digestion actually removed the 200 bp TALEN right homology arm from the whole plasmid.

As we had the Pgk-Puro+SV40 pA segment, our next step was to flank this segment with 1026 bp CRISPR left homology arm and 1029 bp CRISPR right homology arm.

We first amplified the 1029 bp CRISPR right homology arm as a part of 1039 bp fragment from L81 DM1 iPS genomic DNA using the forward primer CCCGTCTGTCGACCTGCTGCTGGGGG (with SalI site; SEQ ID NO: 67) and reverse primer CCCTGGTACCGACTAAGGGCGCGAAG (with KpnI site, SEQ ID NO:68). This fragment was digested (KpnI/SalI) and cloned into the KpnI/SalI digested Pgk-Puro+SV40 pA segment containing backbone.

The good clones were confirmed by sequencing using the following primers

Primer 2 in PGK promoter -

(SEQ ID NO: 69)

CTAAGCTTGGCTGGACGTA
Primer 1 in 1039bp fragment -

(SEQ ID NO: 70)

CCTATGGAAAAACGCCAGC

This product was A837pPGK-PURO-SK, containing the Pgk-Puro+SV40 segment fused with 1029 bp right homology arm which was an intermediate product used to clone our final donor molecule.

The 1026 bp CRISPR left homology arm was then amplified as a part of the 1035 bp fragment from L81 DM1 iPS genomic DNA using the forward primer CCTTGGCGCGCCTCCCTGGCTCCT (with AscI site; SEQ ID NO:71) and reverse primer CCCTGAGCTCCGGCTACAAGGAC (with SacI site; (SEQ ID NO:72).

The intermediate product A837pPGK-PURO-SK was then digested with SacI/AscI to obtain a 5364 bp fragment, which was then ligated to the SacI/AscI digested 1035 bp insert fragment.

The good clones were confirmed by sequencing using the following primers:

Forward primer upstream of 1026bp arm -

(SEQ ID NO: 73)

GATGTGCTGCAAGGCGATTA
Reverse primer after SV40A -

(SEQ ID NO: 74)

| CCACAACTAGAATGCAGTGAAA | |
|---|---|
| SV40-PGK-PURO-DMPK200bp | Plasmid nr 63 |
| A838pPGK-PURO-SKAS | Plasmid nr 66 |

Two days after nucleofection (cf. Example 3), cells were sorted for BFP+ and GFP+cells, which were expanded for 16 days before puromycin selection initiation. Table 9 indicates the transfection conditions as well as the amount and percentage of GFP+, BFP+, and GFP+/BFP+ cells obtained (condition 1A is the experimental condition and condition 2A and 3A are control conditions).

TABLE 9

| | | | | | NO. OF |
|---|---|---|---|---|---|
| | | | | % | GFP+/BFP+ |
| | Plasmid | % GFP | % BFP | GFP+ | SORTED |
| Conditions | Amount | TOTAL | TOTAL | BFP+ | CELLS |
| Condition 1A | | 3.6% | 4.1% | 2.7% | 4000 |
| Cas9-BFP | 1 μg | | | | |
| gRNA CR14189- | 1 μg | | | | |
| GFP | | | | | |
| gRNA CR14254- | 1 μg | | | | |
| GFP | | | | | |
| donor molecule | 2 μg | | | | |
| Condition 2A | | 4.5% | 3% | 2.5% | 12295 |
| CMV-BFP | 0.45 μg | | | | |
| gRNA CR14189- | 1 μg | | | | |
| GFP | | | | | |
| gRNA CR14254- | 1 μg | | | | |
| GFP | | | | | |
| donor molecule | 1 μg | | | | |
| Condition 3A | | 14.4%* | 4.4% | 3.6% | 14200 |
| Cas9-BFP | 1 μg | | | | |
| gRNA CR14189- | 1 μg | | | | |
| GFP | | | | | |
| gRNA CR14254- | 1 μg | | | | |
| GFP | | | | | |
| donor control- | 1.56 μg | | | | |
| dsRED | | | | | |

*the high % maybe an effect of leaking of ds RED signal into GFP channel

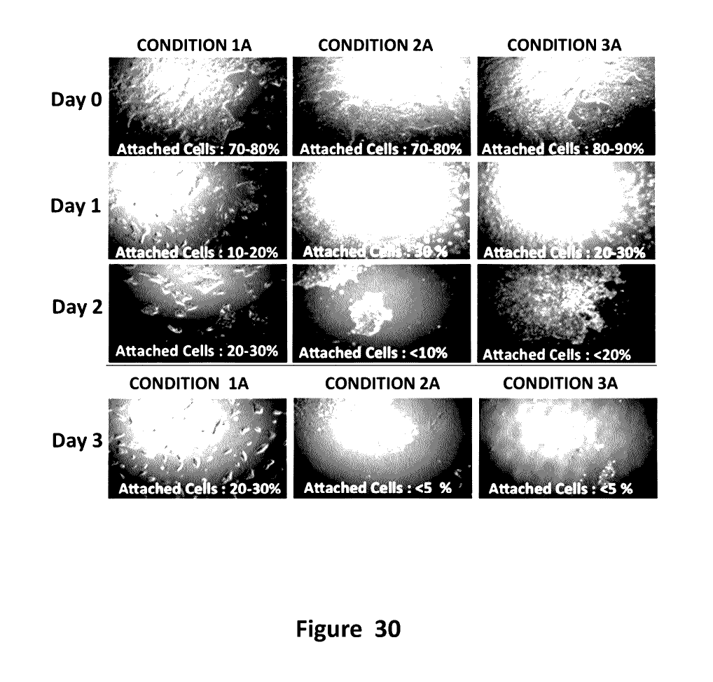

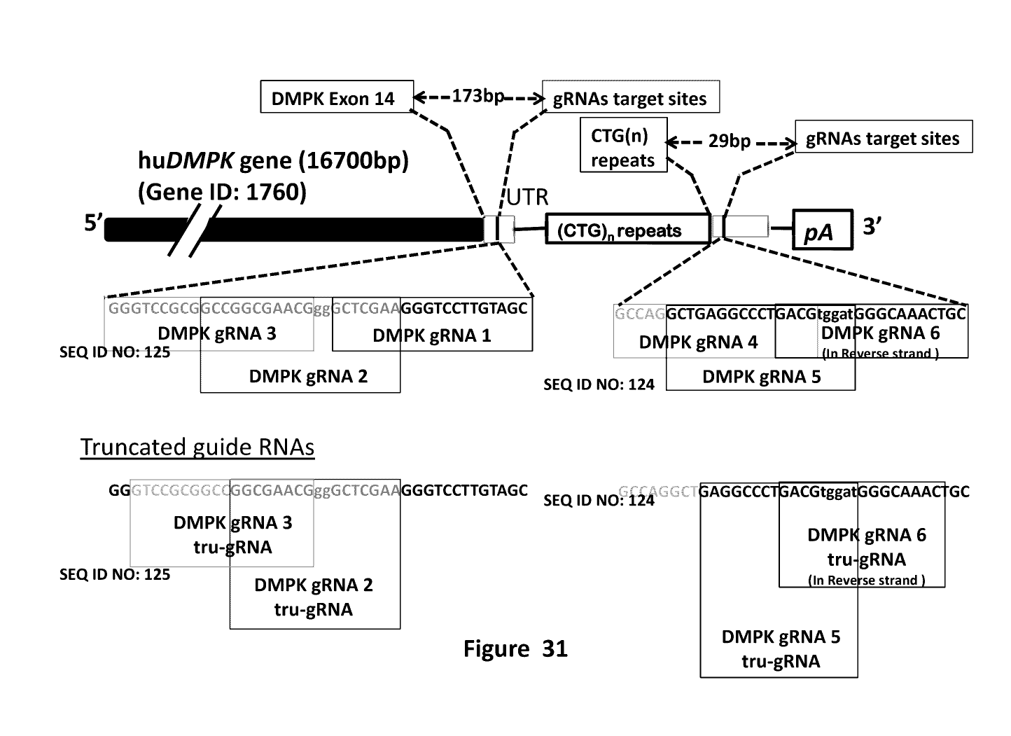

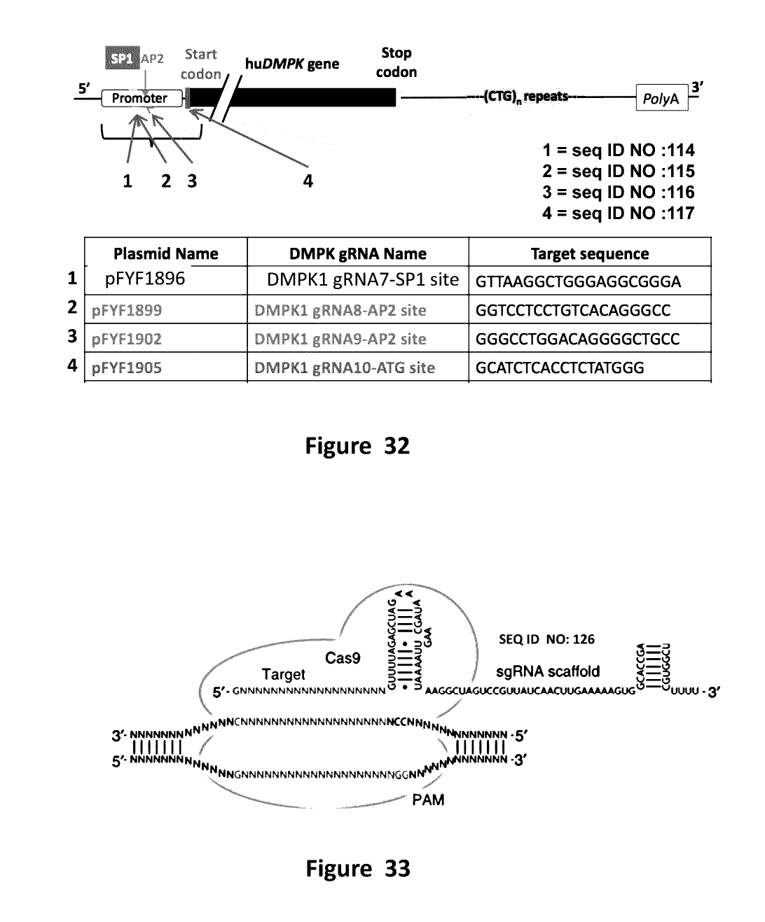

The result of the puromycin-selected transfected cells showed that the DM1-iPS continued to grow after puromycin selection as compared to the control conditions 2A & 3A where almost all the cells were dead 3 days after puro selection (FIG. 30). This indicates successful incorporation of the puromycin cassette from the donor molecule in the genome, likely into the desired locus. This indicates that targeted excision of the CTG repeat had occurred.

### Example 6. Lenti CRISPR Mediated Targeting of the HIDEMs

L81 HIDEM cells (see Example 1) were used for CRISPR/Cas9 mediated targeting. Ire order correct the expanded CTG repeats in these patient cells, CRISPR/Cas genome-editing was performed wherein the Cas9 and gRNA expression cassette were in a lentiviral backbone and were delivered into the HIDEM cells by lentiviral transduction. The donor molecule was delivered by Nucleofection.

A set of guide RNAs was prepared targeting regions near the 5′ end and the 3′ end of the CTG repeat as well as guide RNAs targeting the promoter region of the DMPK gene (at the SP1 and AP2 transcription factor binding site and the ATG start codon).

A set of plasmid vectors comprising a U6 promotor (underlined) different target sequences [target sequence] and the scaffold part of the CRISPR sequence were provided

AGGCTTTAAA GGAACCAATT CAGTCGACTG GATCCGGTAC
CAAGGTCGGG CAGGAAGAGG GCCTATTTCC CATGATTCCT
TCATATTTGC ATATACGATA CAAGGCTGTT AGAGAGATAA
TTAGAATTAA TTTGACTGTA AACACAAAGA TATTAGTACA
AAATACGTGA CGTAGAAAGT AATAATTTCT TGGGTAGTTT
GCAGTTTTAA AATTATGTTT TAAAATGGAC TATCATATGC
TTACCGTAAC TTGAAAGTAT TTCGATTTCT TGGCTTTATA
TATCTTGTGG AAAGGACGAA ACACC[target sequence]
GTTTTAGAGC TAGAAATAGC AAGTTAAAAT AAGGCTAGTC
CGTTATCAAC TTGAAAAAGT GGCACCGAGT CGGTGCTTTT
TTTAAGCTTG GGCCGCTCGA GGTACCTCTC TACATATGAC
ATGTGAGCAA AAGGCCAGCA AAAGGCCAGG AACCGTAAAA
AGGCCGCGTT GCTGGCGT

The sequences 5′ and 3′ of the target sequence are depicted by SEQ ID NO: 87 and SEQ ID NO: 88

The different target sequences which are cloned in the vector are shown in the table 10 below. The table also show the corresponding sequence, including the PAM sequence, in the DMPK genomic sequence:

TABLE 10

| Construct | Target sequence + | Target sequence |
|---|---|---|
| name | PAM | CRIPSR |
| pFYF1884 | GCTCGAAGGGTCCTTGTAG | GCTCGAAGGGTCCTTGTAGC |
| DMD | C CGG | [SEQ ID NO: 104] |
| gRNA 1 | [SEQ ID NO: 89] | |
| 5′ CTG | | |
| repeat | | |
| pFYF1885 | GCCGGCGAACGGGGCTCGA | GCCGGCGAACGGGGCTCGAA |
| DMD | GGG | [SEQ ID NO: 105] |
| gRNA 2 | [SEQ ID NO: 90] | |
| 5′ CTG | | |
| repeat | | |
| pFYF1886 | GGGTCCGCGGCCGGCGAAC | GGGTCCGCGGCCGGCGAACG |
| DMD | G GGG | [SEQ ID NO: 106] |
| gRNA 3 | [SEQ ID NO: 91] | |
| 5′ CTG | | |
| repeat | | |
| pFYF1887 | GCCAGGCTGAGGCCCTGAC | GCCAGGCTGAGGCCCTGACG |
| DMD | G TGG | [SEQ ID NO: 107] |
| gRNA 4 | [SEQ ID NO: 92] | |
| 3′ CTG | | |
| repeat | | |
| pFYF1888 | GCTGAGGCCCTGACGTGGA | GCTGAGGCCCTGACGTGGAT |
| DMD | T GGG | [SEQ ID NO: 108] |
| gRNA 5 | [SEQ ID NO: 93] | |
| 3′ CTG | | |
| repeat | | |
| pFYF1889 | GCAGTTTGCCCATCCACGT | GCAGTTTGCCCATCCACGTC |
| DMD | C AGG | [SEQ ID NO: 109] |
| gRNA 6 | [SEQ ID NO: 94] | |
| 3′ CTG | | |
| repeat | | |
| pFYF1890 | GGCGAACGGGGCTCGAA | GGCGAACGGGGCTCGAA |
| DMD | GGG | [SEQ ID NO: 110] |
| gRNA 2 | [SEQ ID NO: 95] | |
| tru-gRNA | | |
| 5′ CTG | | |
| repeat | | |
| pFYF1891 | GTCCGCGGCCGGCGAACG | GTCCGCGGCCGGCGAACG |
| DMD | GGG | [SEQ ID NO: 111] |
| gRNA 3 | [SEQ ID NO: 96] | |
| tru-gRNA | | |
| 5′ CTG | | |
| repeat | | |
| pFYF1892 | GAGGCCCTGACGTGGAT | GAGGCCCTGACGTGGAT |
| DMD | GGG | [SEQ ID NO: 112] |
| gRNA 5 | [SEQ ID NO: 97] | |
| tru-gRNA | | |
| 3′ CTG | | |
| repeat | | |
| pFYF1881 | GTTTGCCCATCCACGTC | GTTTGCCCATCCACGTC |
| DMD | AGG | [SEQ ID NO: 113] |
| gRNA 6 | [SEQ ID NO: 98] | |
| tru-gRNA | | |
| 3′ CTG | | |
| repeat | | |
| pFYF1896 | GTTAAGGCTGGGAGGCGGG | GTTAAGGCTGGGAGGCGGGA |
| DMPK1 | A GGG | [SEQ ID NO: 114] |
| gRNA7- | [SEQ ID NO: 99] | |
| SP1 | | |
| pFYF1899 | GGTCCTCCTGTCACAGGGC | GGTCCTCCTGTCACAGGGCC |
| DMPK1 | C TGG | [SEQ ID NO: 115] |
| gRNA8- | [SEQ ID NO: 100] | |
| AP2 | | |
| pFYF1902 | GGGCCTGGACAGGGGCTGC | GGGCCTGGACAGGGGCTGCC |
| DMPK1 | C AGG | [SEQ ID NO: 116] |
| gRNA9- | [SEQ ID NO: 101] | |
| AP2 | | |
| pFYF1905 | GCATCTCACCTCTATGGG | GCATCTCACCTCTATGGG |
| DMPK1 | AGG | [SEQ ID NO: 117] |
| gRNA10- | [SEQ ID NO: 102] | |
| ATG | | |
| pFYF1908 | GGCATCTCACCTCTATGGG | GGCATCTCACCTCTATGGGA |
| DMPK1 | A GGG | [SEQ ID NO: 118] |
| gRNA11- | [SEQ ID NO: 103] | |
| ATG | | |

From the above constructs. PCR fragments containing U6 promoter, target sequence and scaffold sequence were generated using a forward primer with a BsWI site and a reverse primer with a SpeI site:

FP =

[SEQ ID NO: 119]

5′-ATCAGCTACGTACGGACTGGATCCGGTACCAAGG-3′
RP =

[SEQ ID NO: 120]

5′GTCGCAGCTAACTAGTCCCAAGCTTAAAAAAAGCACCGA-3′

The PCR fragment was digested with BsWI and SpeI and cloned in a lentiviral vector cut with the same enzymes (VandenDriessche T et al (2002) Bood 100, 813-822.)

### Experimental Design

### Nueleofection of Donor Plasmid

For the in-vitro correction, DM1 iPS derived HIDEMs cells, at passage 8 were used for nucleofection using P1 Primary Cell 4D nucleofected X kit (Lonza). Cells at passage 8 were harvested with 0.05% Trypsin EDTA (Life technologies), and 1×10′ cells were used per nucleofection reaction. The cells were resuspended in 100 μl of nucleofection mixture containing 80 ti of P1 Nucleofector solution, 20 ml of supplement and required DNA (Donor plasmid). Thereafter, we transferred the reaction mixtures into a 100 μl nucleofection cuvette and conducted nucleofection using FF104 program. Cells were divided into 3 single wells of 6 well plate post nucleofection and incubated at 37° C., 5% CO2, 3% 025-6 hrs (Table 11).

TABLE 11
Details of the nucleofection conditions.

| Conditions | Plasmid Amount |
|---|---|
| Condition 1 | |
| Donor Molecule | 12 μg |
| Condition 2 | |
| Donor Molecule | 12 μg |
| Condition 3 | |
| Donor Molecule | 12 μg |
| Condition 4 | |
| No Donor Molecule | — |

Lentiviral Transduction of Cas9 and gRNA

5-6 hours post nucleofection, lentiviral transduction of the Cas9 and CRISPR gRNAs was carried out on these nucleofected cells as described below. HIDEM media with polybrene (8 μg/ml) was prepared and the required concentrated viral amount was added. Media post nucleofection was aspirated out gently and 1 ml of HIDEM media (containing Polybrene and viral particles) was added into each well of 6 well plates and incubated for 16 hours before medium change.

Table 12
conditions of Lentiviral transduction
Cells: HIDEMs clone L81 nucleofected cells
Polybrene: 8 μg/ml
Approach: Targeting of the CTG Repeat Region

| Conditions | Viral Particles | MOI |
|---|---|---|
| Condition 1 | | |
| +Donor Molecule | Cas9 | MOI 25 |
| (Experimental Condition) | gRNA 1885 | MOI 25 |
| | gRNA 1888 | MOI 25 |
| Condition 2 | | |
| +Donor Molecule | Cas9 | MOI 25 |
| (gRNA Control) | Scrambled gRNA | MOI 25 |
| Condition 3 | | |
| +Donor Molecule | gRNA 1885 | MOI 25 |
| (Cas9 Control) | gRNA 1888 | MOI 25 |
| Condition 4 | | |
| No Donor Molecule | Cas9 | MOI 25 |
| (Donor molecule Control) | gRNA 1885 | MOI 25 |
| | gRNA 1888 | MOI 25 |

### Nuclear Foci Staining of CRISPR/Cas Treated HIDEMs Cells

For the Nuclear Foci staining, HIDEMs cells were plated at 40,000 cells per 2.4 cm sq (per chamber) of 4-chambered slide (Lab-Tek® II). Next day the cells were used for Nuclear Foci staining.

### Materials and Methods

For detecting CUGexpRNA Foci (Nuclear Foci), the cells were fixed with 4% PFA for 15 mins and washed 3 times with 70% ethanol (Sigma Aldrich). Following that two 10 mins wash was given with a solution of PBS and 5 mM MgCl2. The cells were then incubated with PNA-5′Cy3 (CAG)5 3′ (Eurogentec) in Hybridization buffer [2×SSC Buffer (Life technologies), 50% Formamide and 0.2% BSA (Sigma Aldrich)] for 90 mins at 37° C. Post hybridization, the cells were washed with PBS (Life technologies)+0.1% Tween (Sigma Aldrich) for 5 mins. Furthermore it was washed in preheated PBS+0.1% Tween for 30 mins at 45° C. Finally the cells were stained with DAPI nuclear stain (1:500, 5 mins) after a PBS wash. Prior to microscopy the cells were again washed with PBS.

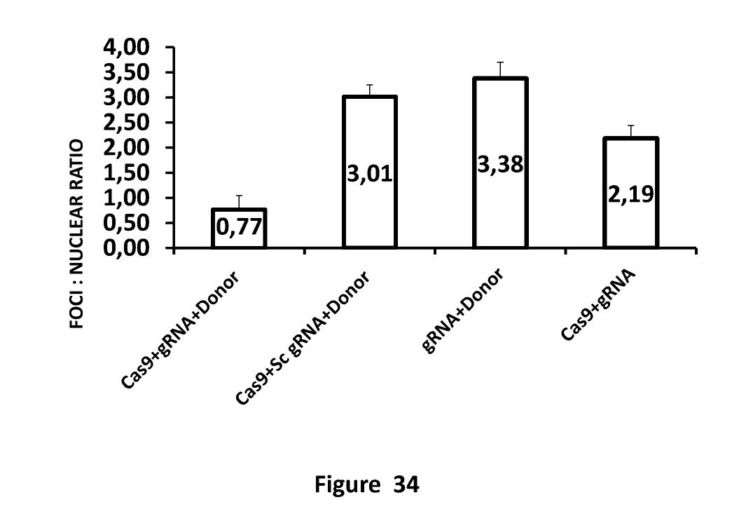

The results of this experiment using the guide RNAs 1885 and 1888 as outlined above show a dramatic reduction of cells with nuclear foci (see table 13 and FIG. 34) which is even more pronounced when a donor molecule is also included.

TABLE 13

| | Cas9 + | Cas9 + | | Cas9 + gRNA |
|---|---|---|---|---|
| | gRNA 1885 | scrambled | gRNA 1885 | 1885 and |
| | and 1888 + | gRNA + | and 1888 + | 1888 (no |
| | donor | donor | donor | donor |
| | molecule | molecule | (no cas9) | molecule) |
| Total Nuclei | 94 | 79 | 50 | 43 |
| NF⁺⁺ + nuclei | 23 | 79 | 50 | 28 |
| NF⁻⁻nuclei | 71 | 0 | 0 | 15 |
| % NF⁻ cells | 76% | — | — | 34% |

1-32. (canceled)

33. A combination of:
a) a polynucleotide for a site specific nuclease targeting the dystrophy myotonic-protein kinase (DMPK) gene locus, and
b) a donor polynucleotide having 5′ and 3′ regions which are homologous with the sequence of DMPK gene which flank the target site of the nuclease defined in a), the combination of polynucleotides being suitable for reducing or eliminating the expression of expanded repeat RNA (CUGexp) of the dystrophy myotonic-protein kinase (DMPK) gene in a cell of a DM-1 patient.

34. The combination of polynucleotides according to claim 33, wherein said polynucleotide for said site specific nuclease is a Clustered Regulatory Interspaced Short Palindromic Repeat (CRISPR) guide RNA of a Cas-based RNA-guided DNA endonuclease.

35. The combination of polynucleotides according to claim 34, further comprising a polynucleotide sequence encoding a Cas9 endonuclease.

36. The combination of polynucleotides according to claim 34, wherein said CRISPR guide RNA and/or said Cas-based RNA-guided DNA endonuclease are comprised within a lentiviral vector.

37. The combination of polynucleotides according to claim 36, wherein said CRISPR guide RNA is capable of specifically binding to the junction between the DMPK gene sequence and the expanded CTG trinucleotide repeat; capable of binding to the SP1 binding site of the DMPK promoter; capable of binding to the AP-2 binding site of the DMPK promoter; or capable of binding to the start codon of the DMPK gene.

38. The combination of polynucleotides according to claim 36, wherein said CRISPR guide RNA is capable of specifically binding at the junction between the DMPK gene sequence and the expanded CTG trinucleotide repeat.

39. The combination of polynucleotides according to claim 37, wherein the target sequence of said CRISPR guide RNA does not overlap with part of said CTG trinucleotide repeat.

40. The combination of polynucleotides according to claim 34, comprising two CRISPR guide RNA molecules, the first one capable of specifically binding at the 5′ junction with the expanded CTG trinucleotide repeat and/or the second one capable of specifically binding at the 3′ junction of the expanded CTG trinucleotide repeat.

41. The combination of polynucleotides to claim 37, comprising two CRISPR guide RNA molecules, the first one capable of specifically binding upstream of the 5′ end of said expanded CTG trinucleotide repeat, and/or the second one capable of specifically binding downstream of the 3′ end of said expanded CTG trinucleotide repeat, wherein the target sequence of said CRISPR guide RNA of one or both guide RNAs does not overlap with part of said CTG trinucleotide repeat.

42. The combination of polynucleotides according to claim 34, wherein the target sequence of said CRISPR guide RNA is between 17 and 20 nucleotides.

43. The combination of polynucleotides according to claim 34, wherein the target sequence of said CRISPR guide RNA sequence has a sequence selected from the group consisting of SEQ ID NO: 50, 51 and 104 to 118, wherein T may be replaced by U.

44. The combination of polynucleotides according to claim 33, wherein said polynucleotide for said site specific nuclease encodes for a Designer Transcription Activator-Like Effector Nuclease (dTALEN).

45. The combination according to claim 44, wherein the sequence coding for the DNA binding part of said dTALEN is depicted by SEQ ID NO:1 or SEQ ID NO:2.

46. The combination of polynucleotides according to claim 33, wherein said donor polynucleotide comprises no protein-encoding sequence inbetween said 5′ and 3′ regions which are homologous with the sequence of DMPK gene which flank the target site.

47. The combination of polynucleotides according to claim 33, wherein said donor polynucleotide comprises at the 5′ end a region which binds the 5′ of the CTG repeat of the DMPK gene and comprises at the 3′ end a region which binds the 3′ of the CTG repeat of the DMPK gene and which comprises in between the two regions 5 to 30 CTG repeats.

48. An in vitro method of reducing or elimination the expression of expanded repeat RNA (CUGexp) of the dystrophy myotonic-protein kinase DMPK gene in cells originating from a subject having myotonic dystrophy type 1 (DM1), comprising the steps of:
a) introducing in said cells a polynucleotide for a site specific nuclease targeting the dystrophy myotonic-protein kinase (DMPK) gene,
b) introducing in said cells a donor polynucleotide having 5′ and 3′ regions which are homologous with the sequence of the DMPK gene which flank the target site of the polynucleotide defined in a).

49. The method according to claim 48, wherein said polynucleotide for a site specific nuclease comprises a polynucleotide for expression of a Cas based RNA-guided DNA endonuclease nuclease and comprises a polynucleotide for the translation of a clustered regulatory interspaced short palindromic repeat (CRISPR) guide RNA for said endonuclease.

50. The method according to claim 48, wherein said polynucleotide for expression of said nuclease, and/or said polynucleotide for translation of said CRISPR guide RNA is a lentiviral vector.

51. The method according to claim 48, where said cells are iPSC or progenitor cells derived thereof.

52. An in vitro method for reducing or eliminating the expression of expanded repeat RNA (CUGexp) of the dystrophy myotonic-protein kinase (DMPK) gene comprising the step of administering the combination of polynucleotides according to claim 33, to a cell originating from a DM-1 patient.

53. A method of reducing the expression of expanded repeat RNA (CUGexp) of the DMPK gene in a subject having myotonic dystrophy type 1 (DM1), comprising the steps of administering to said subject:
a) a polynucleotide for a site specific nuclease targeting the dystrophy myotonic-protein kinase (DMPK) gene,
b) a donor polynucleotide having 5′ and 3′ regions which are homologous with the sequence of DMPK gene which flank the target site of the polynucleotide defined in a).

54. A method of reducing the expression of expanded repeat RNA (CUGexp) of the DMPK gene in a subject having myotonic dystrophy type 1 (DM1), comprising the steps of:
a) isolating cells from said subject and converting said cells to iPS cells,
b) subjecting these cells to a method as defined in claim 48,
c) introducing said cells obtained in step, optionally after differentiation into muscle precursor or progenitor cells, to said subject.

55. A polynucleotide for a CRISPR/Cas comprising a target sequence consisting of a sequence selected from the group consisting of SEQ ID NO: 50, 51 and 104 to 118, or the complement or the reverse complement of said polynucleotide, wherein T may be replaced by U.

56. A vector comprising a polynucleotide according to claim 55.

57. A method of reducing the expression of expanded repeat RNA (CUGexp) of the DMPK gene in a subject having myotonic dystrophy type 1 (DM1), comprising the steps of:
a) isolating cells from said subject and converting said cells to iPS cells,
b) subjecting these cells to a method as defined in claim 52,
c) introducing said cells obtained in step, optionally after differentiation into muscle precursor or progenitor cells, to said subject.

58. A method of reducing the expression of expanded repeat RNA (CUGexp) of the DMPK gene in a subject having myotonic dystrophy type 1 (DM1), comprising the steps of:
a) isolating cells from said subject and converting said cells to iPS cells,
b) subjecting these cells to a method as defined in claim 53,
c) introducing said cells obtained in step, optionally after differentiation into muscle precursor or progenitor cells, to said subject.
